(19) United States
(12) Patent Application Publication
(10) Pub. No.: US 2024/0248099 A1
(43) Pub. Date: Jul. 25, 2024

(54) BIOMARKERS FOR AGE-RELATED MACULAR DEGENERATION

(71) Applicant: BELITE BIO, LLC, San Diego, CA (US)

(72) Inventors: Webber LIAO, San Diego, CA (US); Cheng-Chi Irene WANG, San Diego, CA (US); Yu-Hsin Tom LIN, San Diego, CA (US)

(21) Appl. No.: 18/562,768

(22) PCT Filed: May 20, 2022
(86) PCT No.: PCT/US2022/030313

§ 371 Date: Nov. 20, 2023

Related U.S. Application Data
(60) Provisional application No. 63/191,739, filed on May 21, 2021.

(51) Int. Cl. G01N 33/68; A61K 31/4545; A61P 27/02; C12Q 1/6883; G01N 33/82

(52) U.S. Cl. CPC G01N 33/6893; A61K 31/4545; A61P 27/02; C12Q 1/6883; G01N 33/82; C12Q 2600/118; C12Q 2600/156; G01N 2800/16; G01N 2800/50; G01N 2800/52

(57) ABSTRACT

Provided herein are biomarkers and diagnostics useful in the diagnosis of age-related macular degeneration. Also provided herein are treatments for diseases including age-related macular degeneration.

### CROSS-REFERENCE

This application claims the benefit of U.S. Provisional Application No. 63/191,739 filed May 21, 2021, which is incorporated herein by reference in its entirety.

### BACKGROUND

A need exists in the medicinal arts for the effective risk assessment, diagnosis, and treatment of diseases and disorders associated with retinol-binding protein 4 (RBP4), such as Age-Related Macular Degeneration (AMD).

### BRIEF SUMMARY OF THE INVENTION

Provided herein are biomarkers which can be used to assess the presence of Age-Related Macular Degeneration (AMD) or to assess the risk of developing AMD in an individual. Also provided herein are assays for measuring the biomarkers provided herein. Further provided herein are methods of treating AMD by administering a therapy.

Provided herein are methods for treating age-related macular degeneration in an individual in need thereof comprising: (a) determining by an assay a level of retinol binding protein 4 (RBP4) in a sample from the individual; and (b) if the sample has the level of at least a threshold value of RBP4, then administering a therapy to reduce the level of RBP4 in the individual, wherein the threshold value is at least 25 μg/ml; thereby treating the age-related macular degeneration in the individual. Further provided herein are methods wherein the threshold value of RBP4 is 35 μg/ml. Further provided herein are methods wherein the threshold value of RBP4 is 25-100 μg/ml. Further provided herein are methods wherein the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. Further provided herein are methods wherein the antibody assay comprises ELISA. Further provided herein are methods further comprising: c) determining by a second assay a level of vitamin A in a sample from the individual; and d) if the sample has the level of at least a threshold value of vitamin A, then administering a therapy to reduce the level of vitamin A in the individual, wherein the threshold value of vitamin A is at least 150 ng/mL. Further provided herein are methods wherein the threshold value of vitamin A is about 225 ng/mL or about 390 ng/mL. Further provided herein are methods wherein the threshold value of vitamin A is from 150-500 ng/mL. Further provided herein are methods wherein the second assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. Further provided herein are methods wherein the sample comprises a blood sample. Further provided herein are methods wherein the level is measured from plasma or serum derived from the blood sample. Further provided herein are methods wherein if the level is below the threshold value of RBP4, then providing a recommendation to reassess the individual for macular degeneration after a period of time. Further provided herein are methods wherein the therapy comprises a pharmaceutical composition comprising an RBP4 inhibitor or a compound which reduces blood RBP4 concentration in the individual. Further provided herein are methods wherein the pharmaceutical composition comprises a compound having the structure of Formula (I):

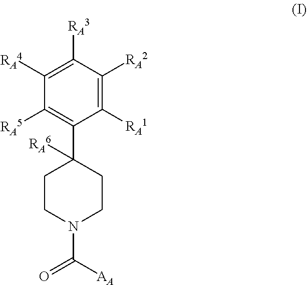

wherein: R_{A}¹, R_{A}², R_{A}³, R_{A}⁴, and R_{A}⁵ are each independently H, halogen, CF₃ or C₁-C₄ alkyl, wherein two or more of R_{A}¹, R_{A}², R_{A}³, R_{A}⁴, and R_{A}⁵ are other than H; R_{A}⁶ is H, OH, or halogen; and A_{A} has the structure:

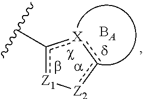

wherein α, β, χ, and δ are each independently absent or present, and when present each is a bond; X is C or N; Z₁ is N; Z₂ is N or NR_{A}⁹, wherein R_{A}⁹ is H, C₁-C₄ alkyl, or oxetane; B_{A} is a substituted or unsubstituted 5, 6, or 7 membered ring structure; or a pharmaceutically acceptable salt thereof. Further rovided herein are methods wherein the compound has the structure

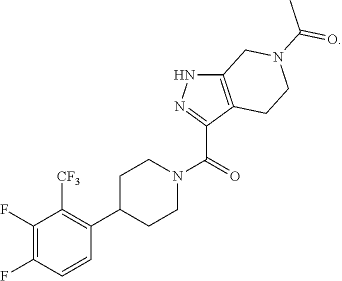 [SMILES: CC(=O)N1CCc2c(C(=O)N3CCC(c4ccc(F)c(F)c4C(F)(F)F)CC3)n[nH]c2C1]

Further provided herein are methods wherein the compound has the structure

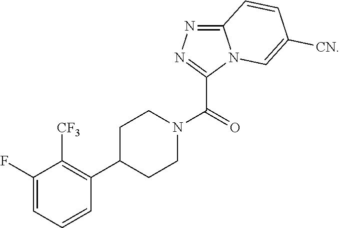 [SMILES: N#Cc1ccc2nnc(C(=O)N3CCC(c4cccc(F)c4C(F)(F)F)CC3)n2c1]

Further provided herein are methods wherein the pharmaceutical composition comprises a compound having the structure of Formula (II):

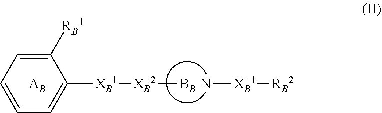

wherein: ring A_{B} is benzene optionally further substituted; R_{B}¹ is an optionally substituted branched C₃-C₆ alkyl group X_{B}¹ is O, S, SO, SO₂ or NH; X_{B}² is a bond or a C₁-C₃ alkylene group; ring B_{B} is azetidine or piperidine; X_{B}³ is CO or SO₂; R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom or a pharmaceutically acceptable salt thereof. Further provided herein are methods wherein the compound is 4-(3-(2-tert-Butylphenoxy)azetidin-1-yl)-4-oxobutanoicacid, 3-{3-[(2-tert-Butyl-4-fluorophenoxy)methyl]azetidin-1-yl}-3-oxopropanoic acid, 2-[3-(2-tert-Butyl-4-chlorophenoxy)azetidin-1-yl]carbonyl pyridine, 4-[3-(2-tert-Butyl-4-chlorophenoxy)azetidin-1-yl]-4-oxobutanoic acid, {3-[(2-tert-Butyl-4-chlorophenoxy)methyl]azetidin-1-yl}(oxo)acetic acid, {3-[(2-tert-butylphenoxy)methyl]azetidin-1-yl}(oxo)acetic acid, 3-(3-[(2-tert-butylphenoxy)methyl]azetidin-1-yl)-3-oxopropanoic acid, [4-[(2-tert-Butyl-4-chlorophenoxy)methyl]piperidin-1-yl](oxo)acetic acid, [4-(2-tert-butylphenoxy)piperidin-1-yl](oxo)acetic acid, or {4-[(2-tert-butylphenoxy)methyl]piperidin-1-yl}(oxo)acetic acid, or a pharmaceutically acceptable salt thereof. Further provided herein are methods wherein the pharmaceutical composition comprises a compound having the structure of Formula (III):

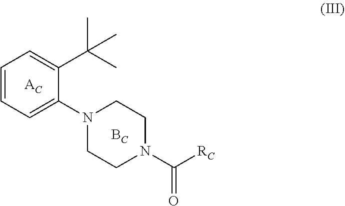

wherein ring A_{C} is a benzene ring optionally substituted by 1 to 3 substituents selected from the group consisting of (a) a halogen atom, and (b) a C₁₋₄ alkyl group; ring B_{C} is a piperazine ring optionally substituted by 1 to 3 substituents selected from the group consisting of (a) a halogen atom, (b) a C₁-C₆alkyl group optionally substituted by 1 to 3 halogen atoms, and (c) a C₁-C₆ alkoxy group optionally substituted by 1 to 3 halogen atoms; and R_{C} is (1) an optionally substituted C₁-C₁₀ alkyl group, (2) an optionally substituted C₆-C₁₄ aryl group, (3) an optionally substituted 5- or 6-membered aromatic heterocyclic group, (4) an optionally substituted amino group, (5) an optionally substituted carboxy group, or (6) an optionally substituted carbamoyl group, or a pharmaceutically acceptable salt thereof. Further provided herein are methods wherein the compound is N-{[4-(2-tert-Butylphenyl)piperazin-1-yl]carbonyl}glycine, 3-[4-(2-tert-Butylphenyl)piperazin-1-yl]-3-oxopropanoic acid, [4-(2-tert-Butyl-4-chlorophenyl)piperazin-1-yl](oxo)acetic acid, 5-(2-[4-(2-tert-Butylphenyl)piperazin-1-yl]-2-oxoethyl)imidazolidine-2,4-dione, [(5-{[4-(2-tert-Butylphenyl)piperazin-1-yl]carbonyl}isoxazol-3-yl)oxy]acetic acid, or a pharmaceutically acceptable salt thereof. Further provided herein are methods wherein the pharmaceutical composition comprises a compound having the structure of Formula (IV):

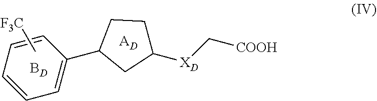

wherein ring A_{D} is a 5-membered non-aromatic heterocycle optionally further substituted by one oxo group; ring B_{D} is a benzene ring optionally further substituted by 1 to 4 substituents; and X_{D} is O, CH₂O, OCH₂, CH₂, (CH₂)₂, S, CH₂S, SCH₂, S(O), CH₂S(O), S(O)CH₂, S(O)₂, CH₂S(O)₂ or S(O)₂CH₂, or a pharmaceutically acceptable salt thereof. Further provided herein are methods wherein the compound is (((3S)-1-[3,5-Bis(trifluoromethyl)phenyl]pyrrolidin-3-yl)oxy)acetic, ({1-[4-Chloro-3-(trifluoromethyl)phenyl]pyrrolidin-3-yl}sulfanyl)acetic acid, 3-{(2R,5S)-5-[3,5-Bis(trifluoromethyl)phenyl]tetrahydrofuran-2-yl}propanoic acid, or a pharmaceutically acceptable salt thereof. Further provided herein are methods wherein the pharmaceutical composition comprises a compound having the structure of Formula (V):

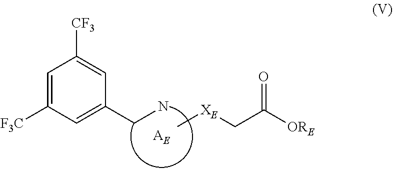

wherein ring A_{E} is a pyrazole ring, a pyridine ring, an oxazole ring, an imidazole ring, or a pyrimidine ring; X_{E} is S, optionally substituted alkylene, or O; and R_{E} is a hydrogen atom or a C₁-C₆ alkyl group; or a pharmaceutically acceptable salt thereof. Further provided herein are methods wherein the compound is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, or 3-(3-(3,5-bis(trifluoromethyl)phenyl)-1H-pyrazol-1-yl)butanoic acid.

Provided herein are methods for treating age-related macular degeneration in an individual in need thereof comprising: determining by an assay a level of vitamin A in a sample from the individual; and if the sample has the level of at least a threshold value of vitamin A, then administering a therapy to reduce the level of vitamin A or a level of RBP4 in the individual, wherein the threshold value is at least 150 ng/ml; thereby treating the age-related macular degeneration in the individual. Further provided herein are methods wherein the threshold value of vitamin A is about 225 ng/mL or about 390 ng/mL. Further provided herein are methods wherein the threshold value of vitamin A is 150-500 ng/mL. Further provided herein are methods wherein the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunobistochemistry assay, or any combination thereof. Further provided herein are methods wherein the chromatographic assay comprises high performance liquid chromatography (HPLC), ultra-high performance liquid chromatography (UPLC), or liquid chromatography/mass spectrometry (LC-MS). Further provided herein are methods wherein the sample comprises a blood sample. Further provided herein are methods wherein the level is measured from plasma or serum derived from the blood sample. Further provided herein are methods wherein if the level is below the threshold value of vitamin A, then providing a recommendation to reassess the individual for macular degeneration after a period of time. Further provided herein are methods wherein the therapy comprises a pharmaceutical composition comprising an RBP4 inhibitor or a compound which reduces blood RBP4 concentration in the individual.

Provided herein are methods for assessing the likelihood of, the severity of, or a diagnosis of age-related macular degeneration in an individual comprising one or more of: a) determining by an assay if a level of retinol binding protein 4 (RBP4) in a sample from the individual is above a threshold level of RBP4; b) determining the age and medical history of the individual; and c) assessing the likelihood of developing, the severity of, or the diagnosis of age related macular degeneration based on the level of RBP4 and optionally on the age and/or medical history of the individual, wherein the threshold level of RBP4 is at least 25 μg/ml. Further provided herein are methods wherein the threshold value is 35 μg/ml. Further provided herein are methods wherein the threshold value is 25-100 μg/ml. Further provided herein are methods wherein the method further comprises performing at least one of color fundus photography, fundus autofluorescence, spectral-domain optical coherence tomography, or microperimetry. Further provided herein are methods further comprising classifying a progression of age-related macular degeneration. Further provided herein are methods wherein classifying comprises use of AREDS categories. Further provided herein are methods wherein the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. Further provided herein are methods wherein the antibody assay comprises ELISA. Further provided herein are methods wherein the sample comprises a blood sample. Further provided herein are methods wherein the level is measured from plasma or serum derived from the blood sample. Further provided herein are methods wherein assessing the likelihood of developing macular degeneration comprises generating a risk score. Further provided herein are methods wherein if the risk score is above a threshold value, administering a pharmaceutical composition for treatment of macular degeneration. Further provided herein are methods further comprising selecting a dose of the pharmaceutical composition based on the level of RBP4. Further provided herein are methods wherein if the risk score is below a threshold value, providing a recommendation to reassess the individual for macular degeneration after a period of time.

Provided herein are methods for treating age-related macular degeneration in an individual in need thereof comprising: a) determining by an assay the presence or absence of one or more genomic variants, wherein the one or more genomic variants comprises at least one of rs4147863, rs2275029, rs1800739, rs4147857, rs4147856, rs1801555, or rs1801574. b) calculating a risk score for age-related macular degeneration using the presence or absence of the one or more genomic variants, and c) administering a therapy to treat age-related macular degeneration in the individual. Further provided herein are methods wherein the one or more genomic variants comprises at least four of rs4147863, rs2275029, rs1800739, rs4147857, rs4147856, rs1801555, or rs1801574. Further provided herein are methods wherein the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281. Further provided herein are methods further comprising determining the age or medical history of the individual. Further provided herein are methods further comprising determining a level of retinol binding protein 4 (RBP4) in a sample from the individual. Further provided herein are methods wherein the therapy comprises administering a pharmaceutical composition to the individual. Further provided herein are methods wherein the pharmaceutical composition comprises an RBP4 inhibitor or a compound which reduces blood RBP4 concentration in the individual. Further provided herein are methods wherein the pharmaceutical composition comprises a compound having the structure of Formula (I):

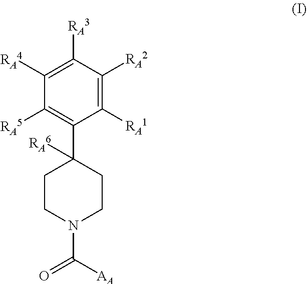

wherein: R_{A}¹, R_{A}², R_{A}³, R_{A}⁴, and R_{A}⁵ are each independently H, halogen, CF₃ or C₁-C₄ alkyl, wherein two or more of R_{A}¹, R_{A}², R_{A}¹, R_{A}⁴, and R_{A}⁵ are other than H, R_{A}⁶ is H, OH, or halogen; and A_{A} has the structure:

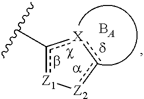

wherein α, β, χ, and δ are each independently absent or present, and when present each is a bond; X is C or N; Z₁ is N; Z₂ is N or NR_{A}⁹, wherein R_{A}⁹ is H, C₁-C₄ alkyl, or oxetane; B_{A} is a substituted or unsubstituted 5, 6, or 7 membered ring structure, or a pharmaceutically acceptable salt thereof. Further provided herein are methods wherein the pharmaceutical composition comprises a compound having the structure

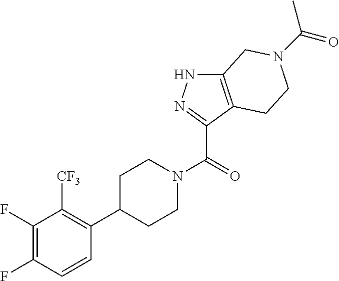 [SMILES: CC(=O)N1CCc2c(C(=O)N3CCC(c4ccc(F)c(F)c4C(F)(F)F)CC3)n[nH]c2C1]

or a pharmaceutically acceptable salt thereof. Further provided herein are methods wherein the pharmaceutical composition comprises a compound having the structure

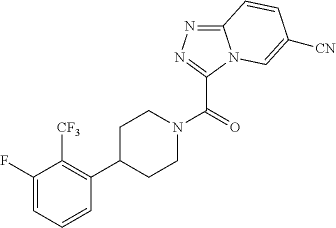 [SMILES: N#Cc1ccc2nnc(C(=O)N3CCC(c4cccc(F)c4C(F)(F)F)CC3)n2c1]

or a pharmaceutically acceptable salt thereof.

Provided herein are methods for assessing the likelihood of age-related macular degeneration in an individual in need thereof comprising: a) extracting a protein fraction from a blood sample obtained the individual; b) extracting a nucleic acid fraction from the blood sample; c) determining by a first assay a level of retinol binding protein 4 (RBP4) from the protein fraction; d) determining by a second assay an allele frequency of one or more genomic variants from nucleic acids in the nucleic acid fraction, wherein the one or more genomic variants comprises at least one of rs4147863, rs2275029, rs1800739, rs4147857, rs4147856, rs1801555, or rs1801574; and e) assessing the likelihood of age-related macular degeneration based on the level of RBP4 and the allele frequency of the one or more genomic variants. Further provided herein are methods wherein the first assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. Further provided herein are methods wherein the antibody assay comprises ELISA Further provided herein are methods wherein the level is measured from plasma or serum derived from the blood sample. Further provided herein are methods wherein the one or more genomic variants comprises at least four of rs4147863, rs2275029, rs1800739, rs4147857, rs4147856, rs1801555, or rs1801574 Further provided herein are methods wherein the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281. Further provided herein are methods further comprising determining the age or medical history of the individual. Further provided herein are methods further comprising performing at least one of color fundus photography, fundus autofluorescence, spectral-domain optical coherence tomography, or microperimetry. Further provided herein are methods further comprising classifying a progression of age-related macular degeneration. Further provided herein are methods wherein classifying comprises use of AREDS categories. Further provided herein are methods wherein assessing the likelihood of age-related macular degeneration comprises generating a risk score.

Provided herein are methods for treating age-related macular degeneration in an individual in need thereof comprising: a) providing a level of retinol binding protein 4 (RBP4), wherein the level was determined by an assay of a protein fraction of a sample from the individual; b) providing an allele frequency of one or more genomic variants, wherein the allele frequency was determined by an assay performed on a nucleic acid fraction of the sample from the individual, and wherein the one or more genomic variants comprises at least one of rs4147863, rs2275029, rs1800739, rs4147857, rs4147856, rs1801555, or rs1801574; and c) administering a therapy based on an assessment of the level of RBP4 and the allele frequency of the one or more genomic variants. Further provided herein are methods wherein the sample comprises a blood sample. Further provided herein are methods wherein the level is measured from plasma or serum derived from the blood sample. Further provided herein are methods wherein the assay is an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. Further provided herein are methods wherein the antibody assay comprises ELISA. Further provided herein are methods wherein the assessment comprises a diagnosis of age-related macular degeneration. Further provided herein are methods wherein the assessment further comprises analysis of at least one of color fundus photography, fundus autofluorescence, spectral-domain optical coherence tomography, or microperimetry. Further provided herein are methods wherein the assessment further comprises analysis of the age or medical history of the individual. Further provided herein are methods wherein the one or more genomic variants comprises at least four of rs4147863, rs2275029, rs1800739, rs4147857, rs4147856, rs1801555, or rs1801574. Further provided herein are methods wherein the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281. Further provided herein are methods wherein the therapy comprises administering a pharmaceutical composition to the individual. Further provided herein are methods wherein the pharmaceutical composition comprises an RBP4 inhibitor or a compound which reduces blood RBP4 concentration in the individual. Further provided herein are methods wherein the pharmaceutical composition comprises a compound having the structure of Formula (I):

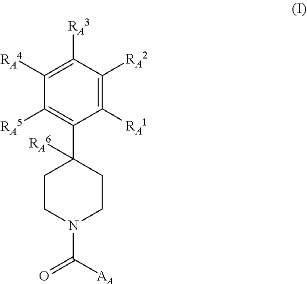

wherein: R_{A}¹, R_{A}², R_{A}³, R_{A}⁴, and R_{A}S are each independently H, halogen, CF₃ or C₁-C₄ alkyl, wherein two or more of R_{A}¹, R_{A}², R_{A}³, R_{A}⁴, and R_{A}⁵ are other than H; R_{A}⁶ is H, OH, or halogen; and A_{A} has the structure:

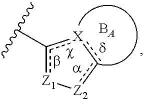

wherein α, β, χ, and δ are each independently absent or present, and when present each is a bond; X is C or N; Z₁ is N; Z₂ is N or NR_{A}⁹, wherein R_{A}⁹ is H, C₁-C₄ alkyl, or oxetane; B_{A} is a substituted or unsubstituted 5, 6, or 7 membered ring structure, or a pharmaceutically acceptable salt thereof. Further provided herein are methods wherein the pharmaceutical composition comprises a compound having the structure

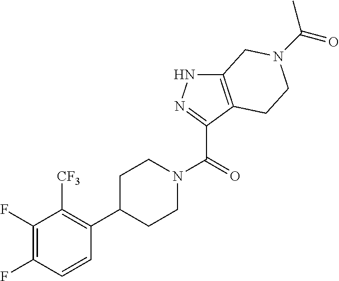 [SMILES: CC(=O)N1CCc2c(C(=O)N3CCC(c4ccc(F)c(F)c4C(F)(F)F)CC3)n[nH]c2C1]

or a pharmaceutically acceptable salt thereof. Further provided herein are methods wherein the pharmaceutical composition comprises a compound having the structure

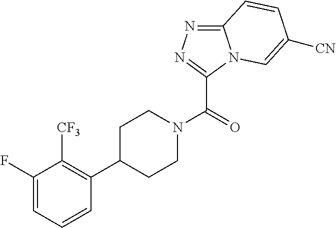 [SMILES: N#Cc1ccc2nnc(C(=O)N3CCC(c4cccc(F)c4C(F)(F)F)CC3)n2c1]

or a pharmaceutically acceptable salt thereof. Further provided herein are methods wherein the pharmaceutical composition comprises a compound having the structure of Formula (II):

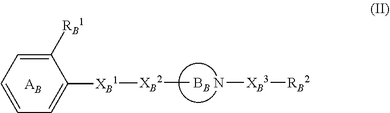

wherein: ring A_{B} is benzene optionally further substituted; R_{B}¹ is an optionally substituted branched C₃-C₆ alkyl group X_{B}¹ is O, S, SO, SO₂ or NH; X_{B}² is a bond or a C₁-C₃ alkylene group; ring B_{B} is azetidine or piperidine; X_{B}³ is CO or SO₂; R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom or a pharmaceutically acceptable salt thereof. Further provided herein are methods wherein the compound is 4-(3-(2-tert-Butylphenoxy)azetidin-1-yl)-4-oxobutanoicacid, 3-{3-[(2-tert-Butyl-4-fluorophenoxy)methyl]azetidin-1-yl}-3-oxopropanoic acid, 2-{[3-(2-tert-Butyl-4-chlorophenoxy)azetidin-1-yl]carbonyl}pyridine, 4-[3-(2-tert-Butyl-4-chlorophenoxy)azetidin-1-yl]-4-oxobutanoic acid, {3-[(2-tert-Butyl-4-chlorophenoxy)methyl]azetidin-1-yl}(oxo)acetic acid, {3-[(2-tert-butylphenoxy)methyl]azetidin-1-yl}(oxo)acetic acid, 3-{3-[(2-tert-butylphenoxy)methyl]azetidin-1-yl}-3-oxopropanoic acid, {4-[(2-tert-Butyl-4-chlorophenoxy)methyl]piperidin-1-yl}(oxo)acetic acid, [4-(2-tert-butylphenoxy)piperidin-1-yl](oxo)acetic acid, or {4-[(2-tert-butylphenoxy)methyl]piperidin-1-yl}(oxo)acetic acid, or a pharmaceutically acceptable salt thereof. Further provided herein are methods wherein the pharmaceutical composition comprises a compound having the structure of Formula (III):

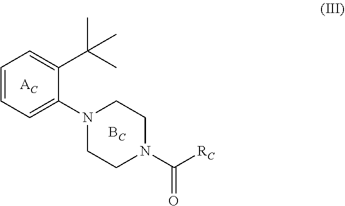

wherein ring A_{C} is a benzene ring optionally substituted by 1 to 3 substituents selected from the group consisting of (a) a halogen atom, and (b) a C₁₋₆ alkyl group; ring B_{C} is a piperazine ring optionally substituted by 1 to 3 substituents selected from the group consisting of (a) a halogen atom, (b) a C₁-C₆alkyl group optionally substituted by 1 to 3 halogen atoms, and (c) a C₁-C₆ alkoxy group optionally substituted by 1 to 3 halogen atoms; and R_{C} is (1) an optionally substituted C₁-C₁₀alkyl group, (2) an optionally substituted C₆-C₁₄ aryl group, (3) an optionally substituted 5- or 6-membered aromatic heterocyclic group, (4) an optionally substituted amino group, (5) an optionally substituted carboxy group, or (6) an optionally substituted carbamoyl group, or a pharmaceutically acceptable salt thereof. Further provided herein are methods wherein the compound is N-{[4-(2-tert-Butylphenyl)piperazin-1-yl]carbonyl}glycine, 3-[4-(2-tert-Butylphenyl)piperazin-1-yl]-3-oxopropanoic acid, [4-(2-tert-Butyl-4-chlorophenyl)piperazin-1-yl](oxo)acetic acid, 5-{2-[4-(2-tert-Butylphenyl)piperazin-1-yl]-2-oxoethyl)imidazolidine-2,4-dione, [(5-([4-(2-tert-Butylphenyl)piperazin-1-yl]carbonyl}isoxazol-3-yl)oxy]acetic acid, or a pharmaceutically acceptable salt thereof. Further provided herein are methods wherein the pharmaceutical composition comprises a compound having the structure of Formula (IV):

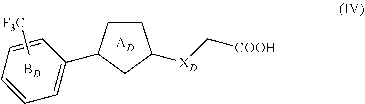

wherein ring A_{D} is a 5-membered non-aromatic heterocycle optionally further substituted by one oxo group; ring B_{D} is a benzene ring optionally further substituted by 1 to 4 substituents; and X_{D} is O, CH₂O, OCH₂, CH₂, (CH₂)₂, S, CH₂S, SCH₂, S(O), CH₂S(O), S(O)CH₂, S(O)₂, CH₂S(O)₂ or S(O)₂CH₂ or a pharmaceutically acceptable salt thereof. Further provided herein are methods wherein the compound is ({(3S)-1-[3,5-Bis(trifluoromethyl)phenyl]pyrrolidin-3-yl}oxy)acetic, ({1-[4-Chloro-3-(trifluoromethyl)phenyl]pyrrolidin-3-yl}sulfanyl)acetic acid, 3-{(2R,5S)-5-[3,5-Bis(trifluoromethyl)phenyl]tetrahydrofuran-2-yl}propanoic acid, or a pharmaceutically acceptable salt thereof. Further provided herein are methods wherein the pharmaceutical composition comprises a compound having the structure of Formula (V):

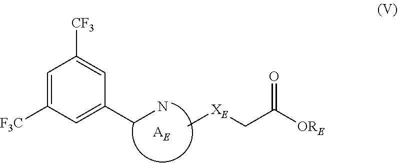

wherein ring A_{E} is a pyrazole ring, a pyridine ring, an oxazole ring, an imidazole ring, or a pyrimidine ring; X_{E} is S, optionally substituted alkylene, or O; and R_{E} is a hydrogen atom or a C₁-C₆ alkyl group, or a pharmaceutically acceptable salt thereof. Further provided herein are methods wherein the compound is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, or 3-(3-(3,5-bis(trifluoromethyl)phenyl)-1H-pyrazol-1-yl)butanoic acid.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a box plot of plasma Retinol binding protein 4 (RBP4) levels in healthy control and different Age-Related Eye Disease Study (AREDS) categories. Plasma RBP4 mean t SD (μg/ml) are 29.4±7.1 (Control), 32.1±6.6 (AREDS2), 37.3±8.8 (AREDS 3), and 36.1±7.8 (AREDS 4). *P<0.05. **P<0.01.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)NOR^{a} (where t is 1 or 2), —S(O)NR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocydyl (optionally substituted with halogen, hydroxy, methoxy, or trifluoromethyl), carbocydylalkyl (optionally substituted with halogen, hydroxy, methoxy, or trifluoromethyl), aryl (optionally substituted with halogen, hydroxy, methoxy, ortrifluoromethyl), aralkyl (optionally substituted with halogen, hydroxy, methoxy, or trifluoromethyl), heterocydyl (optionally substituted with halogen, hydroxy, methoxy, ortrifluoromethyl), heterocyclylalkyl (optionally substituted with halogen, hydroxy, methoxy, or trifluoromethyl), heteroaryl (optionally substituted with halogen, hydroxy, methoxy, or triflucromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)OR^{a} (where t is 1 or 2), —S(O)NR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or triflucromethyl), fluoroalkyl, carbocydyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, ortrifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR² (where t is 1 or 2), —S(O)OR^{a} (where t is 1 or 2), —S(O)ₜNR^{a} (where t is 1 or 2) and —S(O)N(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, ortrifluoromethyl), aryl (optionally substituted with halogen, hydroxy, methoxy, ortrifluoromethyl), aralkyl (optionally substituted with halogen, hydroxy, methoxy, or trifluoromethyl), heterocyclyl (optionally substituted with halogen, hydroxy, methoxy, ortrifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, ortrifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)NR^{a} (where t is 1 or 2), —S(O)NOR^{a} (where t is 1 or 2), —S(O)NR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, ortrifluoromethyl), heterocyclyl (optionally substituted with halogen, hydroxy, methoxy, or trifluoromethyl), heterocyclylalkyl (optionally substituted with halogen, hydroxy, methoxy, ortrifluoromethyl), heteroaryl (optionally substituted with halogen, hydroxy, methoxy, ortrifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₈ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)NR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, ortrifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, ortrifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)NR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, ortrifluoromethyl), cycloalkylalkyl (optionally substituted with halogen, hydroxy, methoxy, ortrifluoromethyl), aryl (optionally substituted with halogen, hydroxy, methoxy, ortrifluoromethyl), aralkyl (optionally substituted with halogen, hydroxy, methoxy, or trifluoromethyl), heterocyclyl (optionally substituted with halogen, hydroxy, methoxy, ortrifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cydohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)R^{a} (where t is 1 or 2), —R^{b}—S(O)NR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, ortrifluoromethyl), aryl (optionally substituted with halogen, hydroxy, methoxy, ortrifluoromethyl), aralkyl (optionally substituted with halogen, hydroxy, methoxy, or trifluoromethyl), heterocyclyl (optionally substituted with halogen, hydroxy, methoxy, ortrifluoromethyl), heterocyclylalkyl (optionally substituted with halogen, hydroxy, methoxy, ortrifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Carbocyclylalkynyl” refers to a radical of the formula —R^{c}-carbocyclyl where R^{c} is an alkynylene chain as defined above. The alkynylene chain and the carbocyclyl radical are optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical are optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

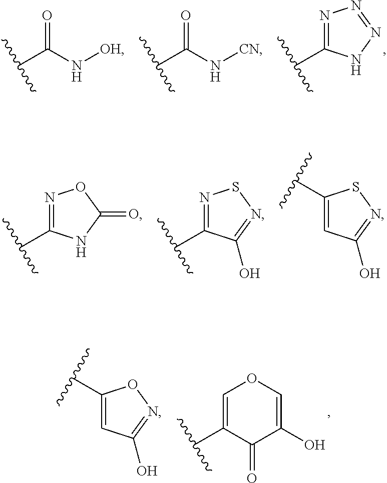

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic ortetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)NOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, ortrifluoromethyl), heterocyclyl (optionally substituted with halogen, hydroxy, methoxy, ortrifluoromethyl), heterocyclylalkyl (optionally substituted with halogen, hydroxy, methoxy, ortrifluoromethyl), heteroaryl (optionally substituted with halogen, hydroxy, methoxy, or trifluoromethyl), or heteroarylalkyl (optionally substituted with halogen, hydroxy, methoxy, ortrifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocydyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocydyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocydyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic ortetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazdyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzrnaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofiranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)R^{a} (where t is 1 or 2), —R^{b}—S(O)ₜR^{a} (where t is 1 or 2), —R^{b}—S(O)NOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, ortrifluoromethyl), heterocyclylalkyl (optionally substituted with halogen, hydroxy, methoxy, ortrifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

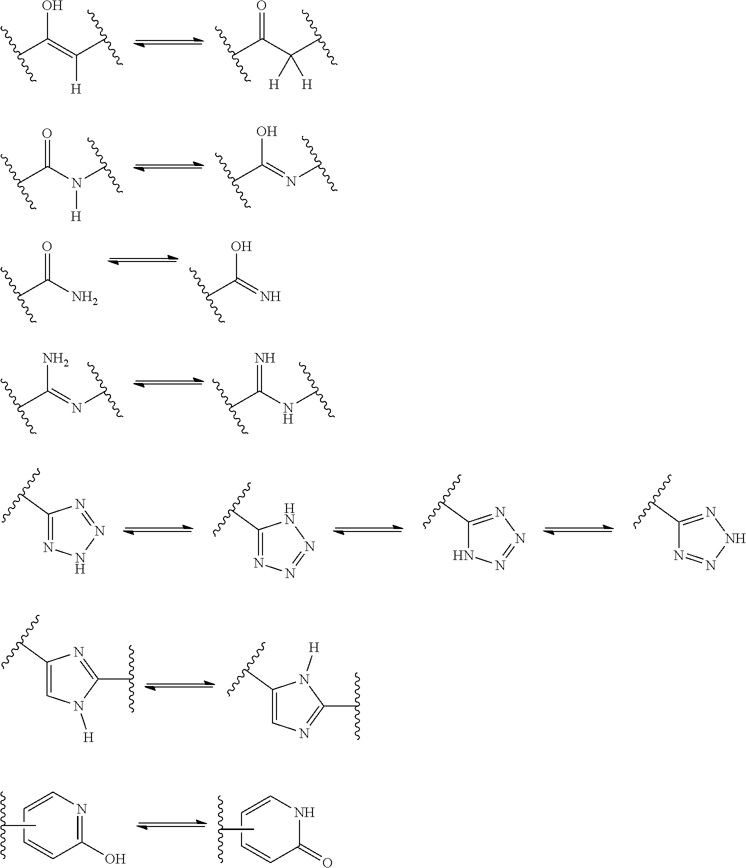

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Ir, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

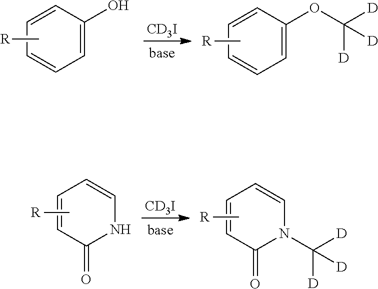

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

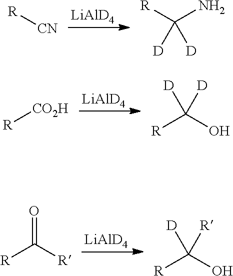

Deuterium gas and palladium catalyst are employed to reduce unsaturated carbon-carbon linkages and to perform a reductive substitution of aryl carbon-halogen bonds as illustrated, by way of example only, in the reaction schemes below.

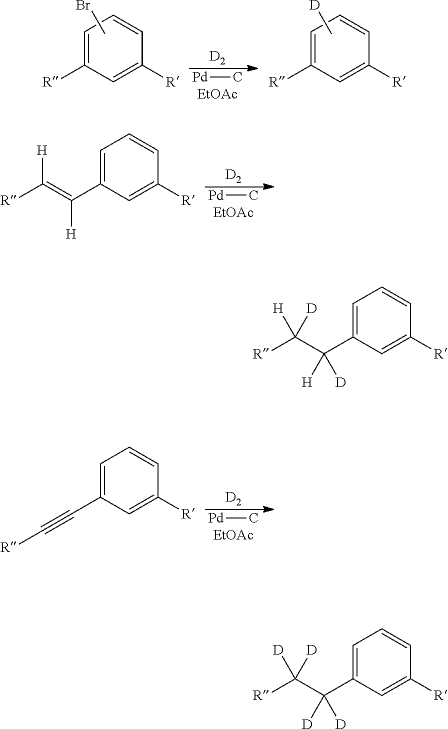

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the heterocyclic RBP4 inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galactronates (see, for example, Berge S M et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., sWpra.

As used herein, “treatment” or “treating,” or“palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

“Prodrug” is meant to indicate a compound that is, in some embodiments, converted under physiological conditions or by solvolysis to a biologically active compound described herein. Thus, the term “prodrug” refers to a precursor of a biologically active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

### Markers of Age-Related Macular Degeneration

Provided herein are method of diagnosing or assessing the risk of developing Age-Related Macular Degeneration in an individual, as well as methods of treatment for the individual and biomarkers relevant to such diagnosis or risk assessment. Age-Related Macular Degeneration (AMD) is a common eye condition and a leading cause of vision loss among people age 50 and older. It causes damage to the macula, a small spot near the center of the retina and the part of the eye needed for sharp, central vision. As AMD progresses, a blurred area near the center of vision is a common symptom. Over time, the blurred area may grow larger and the subject may develop blank spots in his or her central vision.

### Retinol Binding Protein 4 Levels Predictive of AMD

In an aspect provided herein is a method of diagnosing or assessing the risk of developing Age-Related Macular Degeneration (AMD) in an individual in need thereof comprising determining by an assay a level of retinol binding protein 4 (RBP4) in a sample from the individual. In some embodiments, the diagnosing or risk assessment step is accompanied by a treatment step, such as the administration of a therapy to reduce the level of RBP4 in the individual, thereby treating or preventing the AMD in the individual. In some embodiments, the therapy is administered if the level of RBP4 in the sample is above a threshold value (e.g. 25 μg/mL). In some embodiments, the individual is diagnosed or assessed to be at risk of developing AMD if the level of RBP4 in the sample is above a threshold value (e.g. 25 μg/mL).

In another aspect provided herein is a method for assessing the likelihood of age-related macular degeneration in an individual comprising determining by an assay a level of retinol binding protein 4 (RBP4) in a sample from the individual is above a threshold value of RBP4 and assessing the likelihood of developing macular degeneration based on the level of RBP4. In some embodiments, the method also comprises determining the age and/or medical history of the individual. In some embodiments, thus, assessing the likelihood of developing macular degeneration is based on the level of RBP4, age, and medical history of the individual.

In another aspect provided herein is a method for assessing the severity of age-related macular degeneration in an individual comprising determining by an assay a level of retinol binding protein 4 (RBP4) in a sample from the individual is above a threshold value of RBP4 and assessing the severity of the macular degeneration based on the level of RBP4. In some embodiments, the method also comprises determining the age and/or medical history of the individual. In some embodiments, thus, assessing the severity of macular degeneration is based on the level of RBP4, age, and medical history of the individual.

In another aspect provided herein is a method for assessing a diagnosis of age-related macular degeneration in an individual comprising determining by an assay a level of retinol binding protein 4 (RBP4) in a sample from the individual is above a threshold value of RBP4 and assessing the diagnosis of macular degeneration based on the level of RBP4. In some embodiments, the method also comprises determining the age and/or medical history of the individual. In some embodiments, thus, assessing diagnosis of macular degeneration is based on the level of RBP4, age, and medical history of the individual.

In another aspect provided herein is a method for assessing a dosing regimen in an individual with age-related macular degeneration comprising determining by an assay a level of retinol binding protein 4 (RBP4) in a sample from the individual is above a threshold value of RBP4 and assessing the diagnosis of macular degeneration based on the level of RBP4. In some embodiments, the method also comprises determining the age and/or medical history of the individual. In some embodiments, thus, assessing a dosing regimen in an individual with macular degeneration is based on the level of RBP4, age, and medical history of the individual.

In some embodiments, the threshold level of RBP4 is at least 25 μg/mL. In some embodiments, the threshold level of RBP4 is at least 30 μg/mL. In some embodiments, the threshold level of RBP4 is at least 35 μg/mL. In some embodiments, the threshold value is about 25 μg/mL to about 100 μg/mL. In some embodiments, the threshold value is about 25 μg/mL to about 30 μg/mL, about 25 μg/mL to about 35 μg/mL, about 25 μg/mL to about 40 μg/mL, about 25 μg/mL to about 50 μg/mL, about μg/mL to about μg/mL, about μg/mL to about μg/mL, about 25 μg/mL to about 80 μg/mL, about 25 μg/mL to about 90 μg/mL, about 25 μg/mL to about 100 μg/mL, about 30 g/mL to about 35 μg/mL, about 30 μg/mL to about 40 μg/mL, about 30 μg/mL to about 50 μg/mL, about 30 μg/mL to about 60 μg/mL, about 30 μg/mL to about 70 μg/mL, about 30 μg/mL to about 80 μg/mL, about 30 μg/mL to about 90 μg/mL, about 30 μg/mL to about 100 μg/mL, about 35 μg/mL to about 40 μg/mL, about 35 μg/mL to about 50 μg/mL, about 35 μg/mL to about 60 μg/mL, about 35 μg/mL to about 70 μg/mL, about 35 μg/mL to about 80 μg/mL, about 35 μg/mL to about 90 μg/mL, or about 35 μg/mL to about 100 μg/mL. In some embodiments, the threshold value is about 25 μg/mL, about 30 μg/mL, or about 35 μg/mL. In some embodiments, the threshold value is at least about 25 μg/mL, about 30 μg/mL, or about 35 μg/mL. In some embodiments, the threshold value is at most about 30 μg/mL, about 35 μg/mL, about 40 μg/mL, about 50 μg/mL, about 60 μg/mL, about 70 μg/mL, about 80 μg/mL, about 90 μg/mL, or about 100 μg/mL.

In some embodiments, the threshold value may vary based on various characteristics of the individual. Non-limiting examples of such characteristics may include demographic data, gender, age, medical history, previous diagnosis with an eye condition, body-mass index (BMI), height, weight, a systemic condition including obesity, hyperlipidemia, hyperglycemia, hypertension, diabetes, mellitus, cardiovascular disease, or non-alcoholic fatty liver diseases.

In some embodiments, a diagnosis of AMD is made if the level of RBP4 is above the threshold value. In some embodiments, a risk score is calculated if the level of RBP4 is above the threshold value.

In some embodiments, a therapy is administered if the level of RBP4 is above the threshold value. In some embodiments, the therapy is administered to reduce the level of RBP4. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

In some embodiments, an additional step may be taken or recommended if the level of RBP4 is below the threshold value. Such a reading below the threshold value may still indicate that the individual bears some risk of developing AMD in the future and thus future monitoring may be recommended, or additional tests may be needed to confirm or complete a diagnosis of AMD.

Additionally, in some embodiments, the method comprises assessing the likelihood of developing AMD based on the level of RBP4. In some embodiments, assessing the likelihood of developing AMD comprises generating a risk score. In some embodiments, if the risk score is above a threshold value, a treatment of AMD is administered (e.g. treatment with an RBP4 inhibitor). In some embodiments, the risk score reflects the probability that the individual will develop AMD at a point in the future based off the measurements provided herein.

In some embodiments, the method comprises providing a recommendation to reassess the individual for AMD after a period of time (e.g., when a risk score for development of AMD has been calculated or when the level of RBP4 is below the threshold value but there are other indications that AMD may develop). In some embodiments, if the level or the risk score is below the threshold value, the method comprises providing a recommendation to reassess the individual for AMD after a period of time. The period of time may be any suitable period of time. In some embodiments, the period of time is about 1 month to about 24 months. In some embodiments, the period of time is about 1 month to about 3 months, about 1 month to about 6 months, about 1 month to about 9 months, about 1 month to about 12 months, about 1 month to about 18 months, about 1 month to about 24 months, about 3 months to about 6 months, about 3 months to about 9 months, about 3 months to about 12 months, about 3 months to about 18 months, about 3 months to about 24 months, about 6 months to about 9 months, about 6 months to about 12 months, about 6 months to about 18 months, about 6 months to about 24 months, about 9 months to about 12 months, about 9 months to about 18 months, about 9 months to about 24 months, about 12 months to about 18 months, about 12 months to about 24 months, or about 18 months to about 24 months. In some embodiments, the period of time is about 1 month, about 3 months, about 6 months, about 9 months, about 12 months, about 18 months, or about 24 months. In some embodiments, the period of time is at least about 1 month, about 3 months, about 6 months, about 9 months, about 12 months, or about 18 months. In some embodiments, the period of time is at most about 3 months, about 6 months, about 9 months, about 12 months, about 18 months, or about 24 months.

The recommendation to reassess may include the performing an additional test after the period of time, including without limitation reassessing the level of RBP4 in the individual or performing any of the other tests or assessments for AMD provided herein, such as a follow up with a physician for a physical examination of the eye.

In some embodiments, the level of RBP4 in the individual is assessed by an assay. Any suitable assay for measuring the level of RBP4 can be employed (e.g. antibody assay, mass spectrometry based assay (e.g. LC/MS), liquid chromatography assay (e.g. HPLC, UPLC), etc.). In some embodiments, the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. In some embodiments, the assay comprises an antibody assay. In some embodiments, the antibody assay comprises an enzyme-linked immunosorbent assay (ELISA).

In some embodiments, the sample comprises a blood sample from the individual. The blood sample may be further processed, such as to remove impurities or to leave behind the serum and/or plasma. In some embodiments, the sample is a plasma or serum sample. In some embodiments, the sample is a plasma sample. In some embodiments, the sample is a serum sample. In some embodiments, the level is measured from plasma or serum derived from the blood sample. In some embodiments, the level is measured from plasma derived from the blood sample. In some embodiments, the level is measured from serum derived from the blood sample.

In some embodiments, the determination of the diagnosis of AMD, the risk assessment of developing AMD, and/or the decision to administer a therapy is made based on additional considerations in addition to the level of RBP4. In some embodiments, the determination is based on the age and/or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases. In some embodiments, the determination is based at least partially on a level of vitamin A in the individual (e.g., in a blood sample of the individual).

Additionally, the methods provided herein may further comprise any of the additional tests or diagnostics for assessing the presence of AMD in an individual provided herein, including measurement of the presence or absence of any of the genomic variants provided herein or any other measurement which can be used to diagnose AMD. In some embodiments, the method further comprises performing at least one of color fundus photography, fundus autofluorescence, spectral-domain optical coherence tomography, or microperimetry.

In some embodiments, the method further comprises classifying a progression of age-related macular degeneration. The classification can be based on the level of RBP4, measured as provided herein, or in conjunction with other methods provided herein, such as by classification using Age-Related Eye Disease Study (AREDS) classifications.

### Vitamin A Levels Predictive of AMD

In an aspect provided herein is a method of diagnosing or assessing the risk of developing Age-Related Macular Degeneration (AMD) in an individual in need thereof comprising determining by an assay a level of vitamin A in a sample from the individual. In some embodiments, the diagnosing or risk assessment step is accompanied by a treatment step, such as the administration of a therapy to reduce the level of vitamin A or RBP4 in the individual, thereby treating or preventing the AMD in the individual. In some embodiments, the therapy is administered if the level of vitamin A in the sample is above a threshold value (e.g. 150 ng/mL). In some embodiments, the individual is diagnosed or assessed to be at risk of developing AMD if the level of vitamin A in the sample is above a threshold value (e.g. 150 ng/mL).

In another aspect provided herein is a method for assessing the likelihood of age-related macular degeneration in an individual comprising determining by an assay a level of vitamin A in a sample from the individual is above a threshold value of vitamin A and assessing the likelihood of developing macular degeneration based on the level of vitamin A. In some embodiments, the method also comprises determining the age and/or medical history of the individual. In some embodiments, thus, assessing the likelihood of developing macular degeneration is based on the level of vitamin A, age, and medical history of the individual.

In another aspect provided herein is a method for assessing the severity of age-related macular degeneration in an individual comprising determining by an assay a level vitamin A in a sample from the individual is above a threshold value of vitamin A and assessing the severity of the macular degeneration based on the level of vitamin A. In some embodiments, the method also comprises determining the age and/or medical history of the individual. In some embodiments, thus, assessing the severity of macular degeneration is based on the level of vitamin A, age, and medical history of the individual.

In another aspect provided herein is a method for assessing a diagnosis of age-related macular degeneration in an individual comprising determining by an assay a level vitamin A in a sample from the individual is above a threshold value of vitamin A and assessing the diagnosis of macular degeneration based on the level of vitamin A. In some embodiments, the method also comprises determining the age and/or medical history of the individual. In some embodiments, thus, assessing diagnosis of macular degeneration is based on the level of vitamin A, age, and medical history of the individual.

In another aspect provided herein is a method for assessing a dosing regimen in an individual with age-related macular degeneration comprising determining by an assay a level of vitamin A in a sample from the individual is above a threshold value of vitamin A and assessing the diagnosis of macular degeneration based on the level of vitamin A. In some embodiments, the method also comprises determining the age and/or medical history of the individual. In some embodiments, thus, assessing a dosing regimen in an individual with macular degeneration is based on the level of vitamin A, age, and medical history of the individual.

In some embodiments, the threshold level of Vitamin A is at least about 150 ng/mL. In some embodiments, the threshold level of Vitamin A is at least about 175 ng/mL. In some embodiments, the threshold level of Vitamin A is at least about 200 ng/mL. In some embodiments, the threshold level of Vitamin A is at least about 220 ng/mL. In some embodiments, the threshold level of Vitamin A is at least about 221 ng/mL. In some embodiments, the threshold level of Vitamin A is at least about 222 ng/mL. In some embodiments, the threshold level of Vitamin A is at least about 223 ng/mL. In some embodiments, the threshold level of Vitamin A is at least about 224 ng/mL. In some embodiments, the threshold level of Vitamin A is at least about 225 ng/mL. In some embodiments, the threshold level of Vitamin A is at least about 250 ng/mL. In some embodiments, the threshold level of Vitamin A is at least about 300 ng/mL. In some embodiments, the threshold level of Vitamin A is at least about 350 ng/mL. In some embodiments, the threshold lev el of Vitamin A is at least about 390 ng/mL. In some embodiments, the threshold level of Vitamin A is at least about 391 ng/mL. In some embodiments, the threshold level of Vitamin A is at least about 392 ng/mL. In some embodiments, the threshold level of Vitamin A is at least about 393 ng/mL. In some embodiments, the threshold level of Vitamin A is at least about 394 ng/mL. In some embodiments, the threshold level of Vitamin A is at least about 395 ng/mL. In some embodiments, the threshold level of vitamin A is about 150 ng/mL to about 500 ng/mL. In some embodiments, the threshold level of vitamin A is about 150 ng/mL to about 175 ng/mL, about 150 ng/mL to about 200 ng/mL, about 150 ng/mL to about 225 ng/mL, about 150 ng/mL to about 250 ng/mL, about 150 ng/m L to about 300 ng/mL, about 150 ng/mL to about 350 ng/mL, about 150 ng/mL to about 400 ng/mL, about 150 ng/mL to about 450 ng/mL, about 150 ng/mL to about 500 ng/mL, about 175 ng/mL to about 200 ng/mL, about 175 ng/mL to about 225 ng/mL, about 175 ng/mL to about 250 ng/mL, about 175 ng/mL to about 300 ng/mL, about 175 ng/mL to about 350 ng/mL, about 175 ng/mL to about 400 ng/mL, about 175 ng/mL to about 450 ng/mL, about 175 ng/mL to about 500 ng/mL, about 200 ng/mL to about 225 ng/mL, about 200 ng/mL to about 250 ng/mL, about 200 ng/mL to about 300 ng/mL, about 200 ng/mL to about 350 ng/mL, about 200 ng/mL to about 400 ng/mL, about 200 ng/mL to about 450 ng/mL, about 200 ng/mL to about 500 ng/mL, about 225 ng/mL to about 250 ng/mL, about 225 ng/mL to about 300 ng/mL, about 225 ng/mL to about 350 ng/mL, about 225 ng/mL to about 400 ng/mL, about 225 ng/mL to about 450 ng/mL, about 225 ng/mL to about 500 ng/mL, about 250 ng/mL to about 300 ng/mL, about 250 ng/mL to about 350 ng/mL, about 250 ng/mL to about 400 ng/mL, about 250 ng/mL to about 450 ng/mL, about 250 ng/mL to about 500 ng/mL, about 300 ng/mL to about 350 ng/mL, about 300 ng/mL to about 400 ng/mL, about 300 ng/mL to about 450 ng/mL, about 300 ng/mL to about 500 ng/mL, about 350 ng/mL to about 400 ng/mL, about 350 ng/mL to about 450 ng/mL, about 350 ng/mL to about 500 ng/mL, about 400 ng/mL to about 450 ng/mL, about 400 ng/mL to about 500 ng/mL, or about 450 ng/mL to about 500 ng/mL. In some embodiments, the threshold level of vitamin A is about 150 ng/mL, about 175 ng/mL, about 200 ng/mL, about 225 ng/mL, about 250 ng/mL, about 300 ng/mL, about 350 ng/mL, about 400 ng/mL, about 450 ng/mL, or about 500 ng/mL. In some embodiments, the threshold level of vitamin A is at least about 150 ng/mL, about 175 ng/m L, about 200 ng/mL, about 225 ng/mL, about 250 ng/mL, about 300 ng/mL, about 350 ng/mL, about 400 ng/mL, or about 450 ng/mL. In some embodiments, the threshold level of vitamin A is at most about 175 ng/mL, about 200 ng/mL, about 225 ng/mL, about 250 ng/mL, about 300 ng/mL, about 350 ng/mL, about 400 ng/mL, about 450 ng/mL, or about 500 ng/mL.

In some embodiments, the threshold value may vary based on various characteristics of the individual. Non-limiting examples of such characteristics may include demographic data, gender, age, medical history, previous diagnosis with an eye condition, body-mass index (BMI), height, weight, a systemic condition including obesity, hyperlipidemia, hyperglycemia, hypertension, diabetes, mellitus, cardiovascular disease, or non-alcoholic fatty liver diseases.

In some embodiments, the threshold level of vitamin A depends on the age of the individual. In some embodiments, the threshold level of vitamin A is lesser for an adolescent (e.g., an individual with an age of 12-17) than for an adult (e.g., an age of 18 or older). In some embodiments, the threshold level of vitamin A for an adolescent is at least about 150 ng/mL, at least about 175 ng/mL, at least about 200 ng/mL, at least about 210 ng/mL, at least about 220 ng/mL, at least about 221 ng/mL, at least about 222 ng/mL, at least about 223 ng/mL, at least about 224 ng/mL, at least about 225 ng/mL, at least about 230 ng/mL, or at least about 250 ng/mL. In some embodiments, the threshold for an adult is at least about 300 ng/mL, at least about 325 ng/mL, at least about 350 ng/mL, at least about 375 ng/mL, at least about 380 ng/mL, at least about 385 ng/mL, at least about 390 ng/mL, at least about 391 ng/mL, at least about 392 ng/mL, at least about 393 ng/mL, at least about 394 ng/mL, at least about 395 ng/mL, or at least about 400 ng/mL.

In some embodiments, a diagnosis of AMD is made if the level of vitamin A is above the threshold value. In some embodiments, a risk score is calculated if the level of vitamin A is above the threshold value.

In some embodiments, a therapy is administered if the level of vitamin A is above the threshold value. In some embodiments, the therapy is administered to reduce the level of vitamin A. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

In some embodiments, an additional step may be taken or recommended if the level of vitamin A is below the threshold value. Such a reading below the threshold value may still indicate that the individual bears some risk of developing AMD in the future and thus future monitoring may be recommended, or additional tests may be needed to confirm or complete a diagnosis of AMD.

Additionally, in some embodiments, the method comprises assessing the likelihood of developing AMD based on the level of vitamin A. In some embodiments, assessing the likelihood of developing AMD comprises generating a risk score. In some embodiments, if the risk score is above a threshold value, a treatment of AMD is administered (e.g. treatment with an RBP4 inhibitor). In some embodiments, the risk score reflects the probability that the individual will develop AMD at a point in the future based off the measurements provided herein.

In some embodiments, the method comprises providing a recommendation to reassess the individual for AMD after a period of time (e.g., when a risk score for development of AMD has been calculated or when the level of vitamin A is below the threshold value but there are other indications that AMD may develop). In some embodiments, if the level or the risk score is below the threshold value, the method comprises providing a recommendation to reassess the individual for AMD after a period of time. The period of time may be any suitable period of time. In some embodiments, the period of time is about 1 month to about 24 months. In some embodiments, the period of time is about 1 month to about 3 months, about 1 month to about 6 months, about 1 month to about 9 months, about 1 month to about 12 months, about 1 month to about 18 months, about 1 month to about 24 months, about 3 months to about 6 months, about 3 months to about 9 months, about 3 months to about 12 months, about 3 months to about 18 months, about 3 months to about 24 months, about 6 months to about 9 months, about 6 months to about 12 months, about 6 months to about 18 months, about 6 months to about 24 months, about 9 months to about 12 months, about 9 months to about 18 months, about 9 months to about 24 months, about 12 months to about 18 months, about 12 months to about 24 months, or about 18 months to about 24 months. In some embodiments, the period of time is about 1 month, about 3 months, about 6 months, about 9 months, about 12 months, about 18 months, or about 24 months. In some embodiments, the period of time is at least about 1 month, about 3 months, about 6 months, about 9 months, about 12 months, or about 18 months. In some embodiments, the period of time is at most about 3 months, about 6 months, about 9 months, about 12 months, about 18 months, or about 24 months.

The recommendation to reassess may include the performing an additional test after the period of time, including without limitation reassessing the level of vitamin A in the individual or performing any of the other tests or assessments for AMD provided herein, such as a follow up with a physician for a physical examination of the eye.

In some embodiments, the level of vitamin A in the individual is assessed by an assay. Any suitable assay for measuring the level of vitamin A can be employed (e.g., antibody assay, mass spectrometry based assay (e.g. LC/MS), liquid chromatography assay (e.g. HPLC, UPLC), etc.). In some embodiments, the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof. In some embodiments, the assay comprises an antibody assay. In some embodiments, the antibody assay comprises an enzyme-linked immunosorbent assay (ELISA). In some embodiments, the assay is a chromatographic assay. In some embodiments, the assay comprises high performance liquid chromatography (HPLC), ultra-performance liquid chromatography (UPLC), or liquid chromatography/mass spectrometry (LC-MS).

In some embodiments, the sample comprises a blood sample from the individual. The blood sample may be further processed, such as to remove impurities or to leave behind the serum and/or plasma. In some embodiments, the sample is a plasma or serum sample. In some embodiments, the sample is a plasma sample. In some embodiments, the sample is a serum sample. In some embodiments, the level is measured from plasma or serum derived from the blood sample. In some embodiments, the level is measured from plasma derived from the blood sample. In some embodiments, the level is measured from serum derived from the blood sample.

In some embodiments, the determination of the diagnosis of AMD, the risk assessment of developing AMD, and/or the decision to administer a therapy is made based on additional considerations in addition to the level of vitamin A. In some embodiments, the determination is based on the age and/or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases. In some embodiments, the determination is based at least partially on a level of RBP4 in the individual (e.g., in a blood sample of the individual).

Additionally, the methods provided herein may further comprise any of the additional tests or diagnostics for assessing the presence of AMD in an individual provided herein, including measurement of the presence or absence of any of the genomic variants provided herein or any other measurement which can be used to diagnose AMD. In some embodiments, the method further comprises performing at least one of color fundus photography, fundus autofluorescence, spectral-domain optical coherence tomography, or microperimetry.

In some embodiments, the method further comprises classifying a progression of age-related macular degeneration. The classification can be based on the level of vitamin A, measured as provided herein, or in conjunction with other methods provided herein, such as by classification using Age-Related Eye Disease Study (AREDS) classifications.

### Genomic Variants Predictive of AMD

Also provided herein are genomic variants indicative of an individual's propensity to develop AMD. In some embodiments, the genomic variants comprise single nucleotide polymorphisms (SNPs) in a gene encoding ATP-binding cassette, subfamily A, member 4 (ABCA4) in the individual. ABCA4 is a protein encoded by the ABCA4 gene in humans and other eukaryotes. The ABCA4 protein is expressed almost exclusively in the retina and is implicated in Stargardt and other eye diseases, including but not limited to fundus flavimaculatus, cone-rod dystrophy, retinitis pigmentosa, and age-related macular degeneration. Diminished ABCA4 activity is linked with excessive accumulation of toxic retinoids and lipofuscin. Such mutations in some instances are detected by sequencing a subject's DNA or RNA. In some embodiments, the genomic variants provided herein are predictive of a subject's likelihood of developing AMD, either alone or in combination with other factors. The genomic variants herein may comprise SNPs which are missense SNPs, intronic SNPs, synonymous SNPs, or any other type of SNP. Individuals may comprise one or more of the SNPs provided herein, either on the same allele or on different alleles. Examples of such genomic variants and SNPs can be found in Table A below.

TABLE A
Genomic Variants

| Variant | | | | |
|---|---|---|---|---|
| Number | Variant | Mutation | Protein Change | Type |
| 1 | rs3747961 | c.*372= | NA | 3 Prime UTR variant |
| 2 | rs6666652 | c.6764G > T | p.S2255I | Missense |
| 3 | rs1800717 | NA | NA | Intronic |
| 4 | rs763108716 | c.6579C > T | p.F2193= | Synonymous |
| 5 | rs185601596 | NA | NA | Intronic |
| 6 | rs17110761 | NA | NA | Intronic |
| 7 | rs61748519 | c.6255C > T | p.L2085= | Synonymous |
| 8 | rs1801359 | c.6249C > T | p.I2083= | Synonymous |
| 9 | rs145766145 | NA | NA | Intronic |
| 10 | rs76258939 | c.3626T > C | p.M1209T | Missense |
| 11 | rs200551567 | c.2841C > T | p.D947= | Synonymous |
| 12 | rs754765164 | NA | NA | Intronic splice acceptor variant |
| 13 | rs201602424 | c.1614C > T | p.A538= | Synonymous |
| 14 | rs564661476 | NA | NA | Intronic insertion |
| 15 | rs4147831 | c.1269C > T | p.H423= | Synonymous |
| 16 | rs6657239 | c.635G > A | p.R212H | Missense |
| 17 | rs2297632 | NA | NA | Intronic |
| 18 | rs1801555 | c.6285T > C | p.D2095= | Synonymous |
| 19 | rs1762114 | c.6069T > C | p.I2023= | Synonymous |
| 20 | rs4147863^{%} | NA | NA | Intronic |
| 21 | rs2275029^{%} | c.5844A > G | p.P1948= | Synonymous |
| 22 | rs1800739^{%} | NA | NA | Intronic |
| 23 | rs4147857^{%} | c.5814A > G | p.L1938= | Synonymous |
| 24 | rs4147856^{%} | NA | NA | Intronic |
| 25 | rs1801574^{%} | c.5682G > C | p.L1894= | Synonymous |
| 26 | rs55860151 | NA | NA | Intronic |
| 27 | rs1800549 | c.4283C > T | p.T1428M | Missense |
| 28 | rs3112831 | c.1268A > C | p.H423R | Synonymous |
| 29 | rs4147830 | NA | NA | Intronic |
| 30 | rs2297634 | NA | NA | Intronic |
| 31 | rs4847281 | c.141A > G | p.P47= | Synonymous |

In an aspect provided herein is a method of assessing whether an individual has AMD, is likely to develop AMD, and, in some instances, methods of treating or preventing said AMD. In some embodiments, the method comprises determining by an assay the presence or absence of one or more genomic variants. In some embodiments, the one or more genomic variants comprises at least one of rs4147863, rs2275029, rs1800739, rs4147857, rs4147856, rs1801555, or rs1801574. In some embodiments, the method comprises calculating a risk score for age-related macular degeneration using the presence or absence of the one or more genomic variants. In some embodiments, the method comprises diagnosing age-related macular degeneration using the presence or absence of the one or more genomic variants. In some embodiments, the method further comprises administering a therapy to treat age-related macular degeneration in the individual. In some embodiments, the method further comprises administering a therapy to prevent age-related macular degeneration in the individual.

In some embodiments, the method comprises determining by an assay the presence or absence of one or more genomic variants. In some embodiments, the one or more genomic variants comprises at least one of rs4147863, rs2275029, rs1800739, rs4147857, rs4147856, rs1801555, or rs1801574. In some embodiments, the one or more genomic variants comprises at least two of rs4147863, rs2275029, rs1800739, rs4147857, rs4147856, rs1801555, or rs1801574. In some embodiments, the one or more genomic variants comprises at least three of rs4147863, rs2275029, rs1800739, rs4147857, rs4147856, rs1801555, or rs1801574. In some embodiments, the one or more genomic variants comprises at least four of rs4147863, rs2275029, rs1800739, rs4147857, rs4147856, rs1801555, or rs1801574. In some embodiments, the one or more genomic variants comprises at least five of rs4147863, rs2275029, rs1800739, rs4147857, rs4147856, rs1801555, or rs1801574. In some embodiments, the one or more genomic variants comprises at least six of rs4147863, rs2275029, rs1800739, rs4147857, rs4147856, rs1801555, or rs1801574. In some embodiments, the one or more genomic variants comprises each of rs4147863, rs2275029, rs1800739, rs4147857, rs4147856, rs1801555, or rs1801574.

In some embodiments, the one or more genomic variants comprises rs4147863. In some embodiments, the one or more genomic variants comprises rs2275029. In some embodiments, the one or more genomic variants comprises rs1800739. In some embodiments, the one or more genomic variants comprises rs4147857. In some embodiments, the one or more genomic variants comprises rs4147856. In some embodiments, the one or more genomic variants comprises rs1801555. In some embodiments, the one or more genomic variants comprises rs1801574.

In some embodiments, the one or more genomic variants comprises at least one of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281. In some embodiments, the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555,rs1762114, rs55860151, rs1800549, rs3112831, rs4147830, rs2297634, or rs4847281. In some embodiments, the one or more genomic variants comprises at least 1, 2, 3, 4, 5, 6, 7, 8, 9, 10, 11, 12, 13, 14, 15, 16, 17, 18, 19, or 20 of the genomic variants.

In some embodiments, the one or more genomic variants comprises at least 1 genomic variant from Table A. In some embodiments, the one or more genomic variants comprises at least 5 genomic variants from Table A. In some embodiments, the one or more genomic variants comprises at least 7 genomic variants from Table A. In some embodiments, one or more genomic variants comprises at least 10 genomic variants from Table A. one or more genomic variants comprises at least 15 genomic variants from Table A. In some embodiments, the one or more genomic variants comprises at least 1, 2, 3, 4, 5, 6, 7, 8, 9, 10, 11, 12, 13, 14, 15, 16, 17, 18, 19, or 20 of the genomic variants from Table A.

In some embodiments, detecting the presence or absence of the one or more genomic variants further comprises determining the allele frequency of the one or more genomic variants in the individual. In some embodiments, the one or more genomic variants are present on a single copy of the gene in the individual. In some embodiments, the one or more genomic variants are present on a plurality of copies of the gene in the individual.

In some embodiments determining the presence or absence of one or more genomic variants comprises performing an assay on genetic material form the individual. In some cases, the genetic material is obtained from blood, serum, plasma, sweat, hair, tears, urine, and other techniques known by one of skill in the art. In some embodiments, the genetic material is obtained from blood, serum, or plasma of the individual.

In some embodiments, methods of detecting a presence, absence, or level of a genomic variant in the sample obtained from the individual involve detecting a nucleic acid sequence. In some cases, the nucleic acid sequence comprises deoxyribonucleic acid (DNA), such as in the case of detecting complementary DNA (cDNA) of an mRNA transcript. In some instances, the nucleic acid sequence comprises a denatured DNA molecule or fragment thereof. In some instances, the nucleic acid sequence comprises DNA selected from: genomic DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA.

Disclosed herein, in some embodiments, the genomic variant is detected by subjecting a sample obtained from the subject to a nucleic acid-based detection assay. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, microarray, or sequencing. In some embodiments, the sequencing technique comprises next generation sequencing. In some embodiments, the methods involve a hybridization assay such as fluorogenic qPCR (e.g., TaqMan™, SYBR green, SYBR green I, SYBR green II, SYBR gold, ethidium bromide, methylene blue, Pyronin Y, DAPI, acridine orange, Blue View or phycoerythrin), which involves a nucleic acid amplification reaction with a specific primer pair, and hybridization of the amplified nucleic acid probes comprising a detectable moiety or molecule that is specific to a target nucleic acid sequence. In some instances, a number of amplification cycles for detecting a target nucleic acid in a qPCR assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles. In some instances, hybridization may occur at standard hybridization temperatures, e.g., between about 35° C. and about 65° C. in a standard PCR buffer.

An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more gene products described herein (of the PRS). In some instances, the nucleic acid probe specific to a biomarker comprises a nucleic acid probe sequence sufficiently complementary to the polynucleotide sequence of the biomarker. In some instances, the biomarker comprises a transcribed polynucleotide sequence (e.g., RNA, cDNA). In some embodiments, the nucleic acid probe can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the target nucleic acid sequence. In some embodiments, the target nucleic acid sequence is immobilized on a solid surface and contacted with a probe, for example by running the isolated target nucleic acid sequence on an agarose gel and transferring the target nucleic acid sequence from the gel to a membrane, such as nitrocellulose. In some embodiments, the probe(s) are immobilized on a solid surface, for example, in an Affymetrix gene chip array, and the probe(s) are contacted with the target nucleic acid sequence.

In some embodiments, the term “probe” with regards to nucleic acids, refers to any nucleic acid molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxaazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine(R6G), N,N,N; N′-tetramethyl-6-carboxyrhodamine(TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalenesulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine3 (Cy3), indodicarbocyanine 5 (Cy5), indodicarbocyanine 5.5(Cy5.5), 3-(-carboxy-pentyl)-3′-ethyl-5,5′-dimethyloxacarbocyanine (CyA); 1H, 5H, 11H, 15H-Xantheno[2,3,4-ij: 5,6,7-ij]diquinolizin-18-ium, 9-[2 (or4)-[[[6-[2,5-dioxo-1-pyrrolidinyl)oxy]-6-oxohexyl]amino]sulfonyl]-4 (or 2)-sulfophenyl]-2,3,6,7,12,13,16,17-octahydro-inner salt (TR or Texas Red); or BODIPY™ dyes. In some cases, the probe comprises FAM as the dye label.

In some embodiments, detecting the one or more genomic variants comprises sequencing genetic material obtained from a sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some embodiments, a therapy is administered if the genomic variants provided herein are identified in the individual. In some embodiments, the therapy is administered to reduce the level of RBP4 in the individual. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

Additionally, the methods provided herein may further comprise any of the additional tests or diagnostics for assessing the presence of AMD in an individual provided herein, including measurements of the level of RBP4 and comparison to a threshold value as provided herein, measurements of the level of vitamin A and comparison to a threshold value as provided herein, or any other measurement which can be used to diagnose AMD. In some embodiments, the method further comprises performing at least one of color fundus photography, fundus autofluorescence, spectral-domain optical coherence tomography, or microperimetry.

In some embodiments, the method also comprises determining the age and medical history of the individual. In some embodiments, thus, assessing the likelihood of developing macular degeneration is based on presence of the one or more genomic variants, age, and medical history of the individual.

In some embodiments, the determination of the diagnosis of AMD, the risk assessment of developing AMD, or the decision to treat is made based on additional considerations in addition to the presence or absence of the one or more genomic variants. In some embodiments, the determination is based on the age and/or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases.

In some embodiments, identification of the one or more genomic variants provided herein in an individual does not necessitate immediate intervention with a therapy, but may prompt a treating physician to recommend increased monitoring for the development of AMD or related symptoms indicative of the development of AMD. In some embodiments, the method comprises providing a recommendation to reassess the individual for AMD after a period of time. In some embodiments, if the level or the risk score is below the threshold value, the method comprises providing a recommendation to reassess the individual for AMD after a period of time. The period of time may be any suitable period of time. In some embodiments, the period of time is about 1 month to about 24 months. In some embodiments, the period of time is about 1 month to about 3 months, about 1 month to about 6 months, about 1 month to about 9 months, about 1 month to about 12 months, about 1 month to about 18 months, about 1 month to about 24 months, about 3 months to about 6 months, about 3 months to about 9 months, about 3 months to about 12 months, about 3 months to about 18 months, about 3 months to about 24 months, about 6 months to about 9 months, about 6 months to about 12 months, about 6 months to about 18 months, about 6 months to about 24 months, about 9 months to about 12 months, about 9 months to about 18 months, about 9 months to about 24 months, about 12 months to about 18 months, about 12 months to about 24 months, or about 18 months to about 24 months. In some embodiments, the period of time is about 1 month, about 3 months, about 6 months, about 9 months, about 12 months, about 18 months, or about 24 months. In some embodiments, the period of time is at least about 1 month, about 3 months, about 6 months, about 9 months, about 12 months, or about 18 months. In some embodiments, the period of time is at most about 3 months, about 6 months, about 9 months, about 12 months, about 18 months, or about 24 months. The period of time may also be a longer time, such as at least about 2 years, at least about 3 years, at least about 5 years, at least about 10 years, or longer. In some embodiments, it may be recommended that the individual is monitored periodically for development of AMD.

The recommendation to reassess may include performing an additional test after the period of time, including without limitation assessing the level of RBP4 in the individual or performing any of the other tests or assessments for AMD provided herein, such as a follow up with a physician for a physical examination of the eye.

Additionally, the methods provided herein may further comprise any of the additional tests or diagnostics for assessing the presence of AMD in an individual provided herein, including measurement of the level of RBP4 and comparison to a threshold value as provided herein, measurement of the level of vitamin A and comparison to a threshold value as provided herein, or any other measurement which can be used to diagnose AMD. In some embodiments, the method further comprises performing at least one of color fundus photography, fundus autofluorescence, spectral-domain optical coherence tomography, or microperimetry.

In some embodiments, the method further comprises classifying a progression of age-related macular degeneration. The classification can be based on the level of RBP4, measured as provided herein, or in conjunction with other methods provided herein, such as by classification using Age-Related Eye Disease Study (AREDS) classifications.

### Combination of RBP4 Level/Vitamin a Level and Genomic Variants Predictive of AMD

The biomarker levels (e.g. blood concentration of RBP4 and/or vitamin A) and genomic variants (e.g. ABCA4 variants) provided herein may also be probed simultaneously in order to provide a diagnosis, risk score, or recommendation for treatment of AMD in an individual. In some embodiments, this combinatorial approach yields a diagnosis or risk score that is more reliable than either method alone. In some embodiments, this allows for a certain diagnosis or risk assessment of AMD without a more invasive physical examination of the eye, as the diagnosis or risk score can be calculated from a single sample from the individual (e.g. a blood sample).

In one aspect herein is a method for assessing the likelihood of age-related macular degeneration in an individual in need thereof comprising: obtaining a blood sample from the individual; extracting a protein fraction from the blood sample; extracting a nucleic acid fraction from the blood sample; determining by a first assay a level of retinol binding protein 4 (RBP4) and/or a level of vitamin A from the protein fraction; determining by a second assay an allele frequency of one or more genomic variants from nucleic acids in the nucleic acid fraction; and assessing the likelihood of age-related macular degeneration based on the level of RBP4 and/or the level of vitamin A and the allele frequency of the one or more genomic variants. In some embodiments, the one or more genomic variants comprises at least one of rs4147863, rs2275029, rs1800739, rs4147857, rs4147856, rs1801555, or rs1801574.

In another aspect herein is a method for treating age-related macular degeneration in an individual in need thereof comprising: a) providing a level of retinol binding protein 4 (RBP4) and/or a level of vitamin A, wherein the level was determined by an assay of a protein fraction of a sample from the individual; b) providing an allele frequency of one or more genomic variants, wherein the allele frequency was determined by an assay performed on a nucleic acid fraction of the sample from the individual; and c) administering a therapy based on an assessment of the level of RBP4 and/or a level of vitamin A and the allele frequency of the one or more genomic variants. In some embodiments, the one or more genomic variants comprises at least one of rs4147863, rs2275029, rs1800739, rs4147857, rs4147856, rs1801555, or rs1801574.

In some embodiments, the level of RBP4 is compared to a threshold value of RBP4 as provided elsewhere herein. Any of the threshold values of RBP4 provided herein may be used in diagnostic/assessment protocols in conjunction with the detection of genomic variants. Additionally, any of the methods or assays used to determine the level of RBP4 in the sample provided herein are equally applicable to methods which base the diagnosis of AMD, the assessment of risk of developing AMD, or the decision to treat AMD on combinations of genomic variants and RBP4 level.

In some embodiments, the methods herein provide for detecting the presence or absence of the one or more genomic variants. Assessment of any combination or number of genomic variants provided herein can be used in conjunction with the information provided by the level of RBP4 in making the diagnosis, risk assessment, or treatment of AMD provided herein.

In embodiments where both RBP4 levels and genomic variants are measured, the measuring of RBP4 levels by an assay and the determining by a second assay the presence or absence of one or more genomic variants (or the allele frequency thereof) are perfonmed on samples derived from the individual. In some embodiments, the assay and the second assay are performed on the same sample. In some embodiments, the assay and the second assay are performed on different samples derived from the individual. The samples need not be taken from the individual at the same time, nor do the samples need to be of the same type. In some embodiments, the assay and the second assay are performed on the same sample derived from the individual. In some embodiments, the assay and the second assay are performed on two separate samples from the individual. In some embodiments, the two separate samples are the same type of sample (e.g. blood samples). In some embodiments, the two separate samples are different types of samples (e.g. a blood sample and a urine sample). In some embodiments, the two separate samples are two blood samples. The two separate samples need not be taken from the individual at the same time. In some embodiments, the two separate samples are taken from the individual at the same time. In some embodiments, the two separate samples are taken at different times. In some embodiments, the two separate samples are taken at most about 1 day, at most about 2 days, at most about 3 days, at most about 1 week, at most about 2 weeks, at most about 1 month, at most about 2 months, at most about 3 months, or at most about 6 months apart.

Just as methods provided herein based on a measurement RBP4 level or the presence or absence of genomic variants may further comprise additional tests or diagnostics to ascertain the presence of AMD in an individual, so too may methods which utilize both RBP4 level and the presence or absence of genomic variants. In some embodiments, the diagnosis, risk assessment, or decision to administer a therapy is further based on additional criteria, such as the age and medical history of the subject. In some embodiments, the determination is based on the age and/or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases.

Additionally, the methods provided herein may further comprise any of the additional tests or diagnostics for assessing the presence of AMD in an individual provided herein. In some embodiments, the method further comprises performing at least one of color fundus photography, fundus autofluorescence, spectral-domain optical coherence tomography, or microperimetry.

In some embodiments, the method further comprises classifying a progression of age-related macular degeneration. The classification can be based on the level of RBP4, measured as provided herein, or in conjunction with other methods provided herein, such as by classification using Age-Related Eye Disease Study (AREDS) classifications.

In some embodiments, the risk score reflects the probability that the individual will develop AMD at a point in the future based off the measurements provided herein.

In some embodiments, identification of the one or more genomic variants provided herein and measurement of the level or RBP4 in an individual does not necessitate immediate intervention with a therapy, but may prompt a treating physician to recommend increased monitoring for the development of AMD or related symptoms indicative of the development of AMD. In some embodiments, the method comprises providing a recommendation to reassess the individual for AMD after a period of time. In some embodiments, if the level or the risk score is below the threshold value, the method comprises providing a recommendation to reassess the individual for AMD after a period of time. The period of time may be any suitable period of time. In some embodiments, the period of time is about 1 month to about 24 months. In some embodiments, the period of time is about 1 month to about 3 months, about 1 month to about 6 months, about 1 month to about 9 months, about 1 month to about 12 months, about 1 month to about 18 months, about 1 month to about 24 months, about 3 months to about 6 months, about 3 months to about 9 months, about 3 months to about 12 months, about 3 months to about 18 months, about 3 months to about 24 months, about 6 months to about 9 months, about 6 months to about 12 months, about 6 months to about 18 months, about 6 months to about 24 months, about 9 months to about 12 months, about 9 months to about 18 months, about 9 months to about 24 months, about 12 months to about 18 months, about 12 months to about 24 months, or about 18 months to about 24 months. In some embodiments, the period of time is about 1 month, about 3 months, about 6 months, about 9 months, about 12 months, about 18 months, or about 24 months. In some embodiments, the period of time is at least about 1 month, about 3 months, about 6 months, about 9 months, about 12 months, or about 18 months. In some embodiments, the period of time is at most about 3 months, about 6 months, about 9 months, about 12 months, about 18 months, or about 24 months. The period of time may also be a longer time, such as at least about 2 years, at least about 3 years, at least about 5 years, at least about 10 years, or longer. In some embodiments, it may be recommended that the individual is monitored periodically for development of AMD.

The recommendation to reassess may include the performing an additional test after the period of time, including without limitation assessing the level of RBP4 in the individual or performing any of the other tests or assessments for AMD provided herein, such as a follow up with a physician for a physical examination of the eye.

In some embodiments, a therapy is administered if the genomic variants provided herein are identified in the individual. In some embodiments, the therapy is administered to reduce the level of RBP4 in the individual. In some embodiments, the therapy comprises administering a pharmaceutical composition to the individual. In some embodiments, the therapy comprises administering a pharmaceutical compound to the individual. In some embodiments, the pharmaceutical composition comprises an RBP4 inhibitor. The RBP4 inhibitor may be any of the RBP4 inhibitors provided herein.

### Additional Considerations for Prediction or Diagnosis of AMD

In some embodiments, the determination of the diagnosis of AMD, the risk assessment of developing AMD, or the decision to treat is made based on additional tests which can optionally be performed in addition to measuring the level of RBP4 in the subject and/or determining the presence or absence of the genomic variants provided herein. Thus, the methods provided herein may further provide additional tests used in conjunction with the biomarkers indicative of AMD provided herein.

The methods provided herein may further comprises additional tests used to assess the presence or risk of developing AMD in an individual. A variety of such additional tests and diagnoses are known in the art, including color fundus photograph, fundus auto fluorescence, spectral-domain optical coherence tomography, and microperimetry.

In some embodiments, the methods provided herein further comprise analysis of at least one of color fundus photography, fundus autofluorescence, spectral-domain optical coherence tomography, or microperimetry. In some embodiments, the method further comprises analysis of color fundus photography. In some embodiments, the method further comprises analysis offundus autofluorescence. In some embodiments, the method further comprises spectral-domain optical coherence tomography. In some embodiments, the method further comprises microperimetry. In some embodiments, the diagnosis of AMD, the risk assessment of developing AMD, or the decision to administer to a therapy for AMD is based at least partially on one of these assessments.

Additionally, the methods provided herein may also comprise determining the age or medical history of the individual. In some embodiments, the determination is based at least partially on the age of the individual. In some embodiments, the determination is based at least partially on the medical history of the individual. In some embodiments, the medical history of the individual can include information such as previous diagnosis with an eye condition, body-mass index (BMI), height, weight, systemic condition including obesity, hyperlipidemia, hyperglycemia, hypertension, diabetes mellitus, cardiovascular disease, or non-alcoholic fatty liver diseases.

In some embodiments, the methods herein comprise classifying progression of the AMD. In some embodiments, the classification is based on the Age-Related Eye Disease Study Research categories [Age-Related Eye Disease Study Research G. The Age-Related Eye Disease Study (AREDS): design implications. AREDS report no. 1. Control Clin Trials. 1999; 20(6):573-600) (AREDS)]. AREDS categories include category 1 (defined as a few (1-15), small (<63 μm), or no drusen and without pigment changes); category 2 (early AMD characterized by several small, few intermediate-sized (63-124 μm) drusen, and/or pigmentary changes in one or both eyes); category 3 (intermediate AMD characterized by extensive (20 soft or 65 hard without any soft) intermediate-size drusen, one large (>125 μm) drusen, and/or geographic atrophy not involving the macula in one or both eyes); and category 4 (advanced unilateral AMD consisting of the advanced dry form with geography atrophy involving macula or the exudative form with choroidal neovascularization in one eye).

RBP4 Inhibitory Compounds and Compounds which Lower RBP4 in the Blood

Provided herein in some embodiments are RBP4 inhibitory compounds, compounds which lower RBP4 levels in the blood, and pharmaceutical compositions comprising said compounds. In some embodiments, the compounds are RBP4 inhibitors. In some embodiments, the compounds lower RBP4 levels in the blood. The subject compounds and compositions are useful for inhibiting RPB4 and for the treatment of various disorders, including age-related macular degeneration. In some embodiments, an individual is administered a therapy comprising an RBP4 inhibitory compound, such as those provided herein, upon diagnosis with AMD or an assessment that the individual is at risk of developing AMD with the methods provided herein. Examples of RBP4 inhibitor compounds can be found in US Publication No. US 2010/0292206; PCT Publication No. WO2010119992; US Publication No. US2011/0251187, US Publication No. US2018/0237404; U.S. Pat. Nos. 10,273,243; 8,980,924; 9,637,450; 9,944,644; 9,938,291; 10,072,016; and PCT Publication No. WO2018232154, each of which is incorporated by reference.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (I):

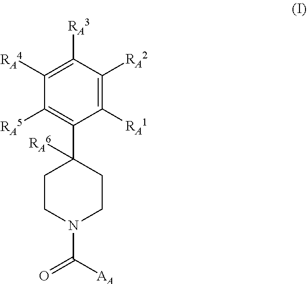

wherein:
   each R_{A}¹, R_{A}², R_{A}³, R_{A}⁴, and R_{A}⁵ is independently halogen, optionally substituted alkyl, optionally substituted cycloalkyl, optionally substituted heterocyclyl, optionally substituted heterocyclolalkyl, —COR_{A}⁷, —CON(R_{A}⁷)₂, optionally substituted (C₀-C₄ alkylene)-CN, optionally substituted (C₀-C₄ alkylene)-OR_{A}⁷, optionally substituted (C₀-C₄ alkylene)-N(R_{A}⁷)₂, optionally substituted (C₀-C₄ alkylene)N(R_{A}⁸)—COR_{A}⁷, optionally substituted (C₀-C₄ alkylene)-SO₂N(R_{A}⁷)₂, optionally substituted (C₀-C₄ alkylene)-SO₂R_{A}⁷, optionally substituted (C₀-C₄ alkylene)N(R_{A}⁸)—SO₂N(R_{A}⁷)₂, or optionally substituted (C₀-C₄ alkylene)N(R_{A}⁸)—SO₂R_{A}⁷;
      each R_{A}⁷ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R_{A}¹¹ groups together with the nitrogen to which they are attached join to form an optionally substituted N-heterocyclyl;
      each R_{A}⁸ is independently selected from H or optionally substituted alkyl;
   R_{A}⁶ is —H, —OH, optionally substituted alkyl, or halogen;
   p is 0, 1, 2, 3, 4, or5;
   A_{A} has the structure:

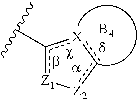

wherein:
   α, β, χ, and δ are each independently absent or present, and when present each is a bond;
   X is C;
   Z₁ is S, O, or N;
   Z₂ is S, O, N, or NR_{A}⁹;
   R_{A}⁹ is H, optionally substituted alkyl, or oxetane; and
   B_{A} is a substituted or unsubstituted fused 5-, 6-, or 7-membered ring structure.

In certain embodiments, the compound of Formula (I) has the structure

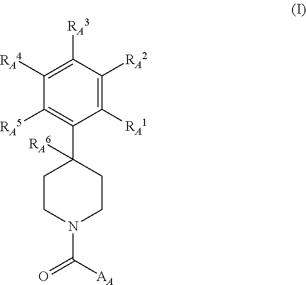

wherein
R_{A}¹, R_{A}², R_{A}³, R_{A}⁴, and R_{A}⁵ are each independently H, halogen, CF₃ or C₁-C₄ alkyl, wherein two or more of R_{A}¹, R_{A}², R_{A}³, R_{A}⁴, and R_{A}⁵ are other than H;
R_{A}⁶ is H, OH, or halogen; and
A_{A} has the structure:

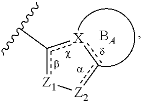

wherein
   α, β, χ, and δ are each independently absent or present, and when present each is a bond;
   X is C or N;
   Z₁ is N;
   Z₂ is N or NR_{A}⁹,
   wherein R_{A}⁹ is H, C₁-C₄ alkyl, or oxetane;
B_{A} is a substituted or unsubstituted 5, 6, or 7 membered ring structure,
or a pharmaceutically acceptable salt thereof.

In some embodiments, the compound of Formula (I) is 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)ethan-1-one. In certain embodiments, the compound of Formula (I) is 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(3,4-difluoro-2-(trifluoromethyl)phenyl)piperidine-1-carbonyl)-4,6-dihydropyrrolo[3,4-c]pyrazol-5(1H)-yl)ethan-1-one; (4-(3-fluoro-2,5-bis(trifluoromethyl)phenyl)piperidin-1-yl)(4,5,6,7-tetrahydro-1H-pyrazolo[4,3-c]pyridin-3-yl)methanone; (4-(2-chloro-3-fluorophenyl)piperidin-1-yl)(5-(2-methoxyethyl)-4,5,6,7-tetrahydro-1H-pyrazolo[4,3-c]pyridin-3-yl)methanone; (4-(2-chloro-3-fluorophenyl)piperidin-1-yl)(5-(3,3,3-trifluoropropyl)-4,5,6,7-tetrahydro-1H-pyrazolo[4,3-c]pyridin-3-yl)methanone; (4-(2-chloro-3-fluorophenyl)piperidin-1-yl)(5-(2,2,2-trifluoroethyl)-4,5,6,7-tetrahydro-1H-pyrazolo[4,3-c]pyridin-3-yl)methanone; (4-(2-chloro-3-fluorophenyl)piperidin-1-yl)(5-(oxetan-3-yl)-4,5,6,7-tetrahydro-1H-pyrazolo[4,3-c]pyridin-3-yl)methanone; (4-(4-fluoro-2-(trifluoromethyl)phenyl)piperidin-1-yl)(4,5,6,7-tetrahydro-1H-pyrazolo[4,3-c]pyridin-3-yl)methanone; (4-(4-fluoro-2-(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(5-(cyclopropylmethyl)-4,5,6,7-tetrahydro-1H-pyrazolo[4,3-c]pyridin-3-yl)methanone; (4-(2-chloro-3-fluorophenyl)piperidin-1-yl)(5-ethyl-4,5,6,7-tetrahydro-1H-pyrazolo[4,3-c]pyridin-3-yl)methanone; (4-(3,4-difluoro-2-(trifluoromethyl)phenyl)piperidin-1-yl)(6-ethyl-4,5,6,7-tetrahydro-1H-pyrazolo[3,4-c]pyridin-3-yl)methanone; (4-(4-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; 1-(3-(4-(2-fluoro-6-(trifluoromethyl)phenyl)piperidine-1-carbonyl)-1,4,5,7-tetrahydro-6H-pyrazolo[3,4-c]pyridin-6-yl)ethan-1-one; (4-(3-fluoro-2,5-bis(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(6-(cyclopropylmethyl)-4,5,6,7-tetrahydro-1H-pyrazolo[3,4-c]pyridin-3-yl)methanone; (4-(3,5-bis(trifluoromethyl)phenyl)piperidin-1-yl)(5-(methylsulfonyl)-4,5,6,7-tetrahydro-1H-pyrazolo[4,3-c]pyridin-3-yl)methanone; (4-(2-chloro-3-fluorophenyl)piperidin-1-yl)(5-(methylsulfonyl)-4,5,6,7-tetrahydro-1H-pyrazolo[4,3-c]pyridin-3-yl)methanone; (4-(2-chloro-3-fluorophenyl)piperidin-1-yl)(6-(oxetan-3-yl)-4,5,6,7-tetrahydro-1H-pyrazolo[3,4-c]pyridin-3-yl)methanone; 3-(4-(2-chloro-3-fluorophenyl)piperidine-1-carbonyl)-1,4,5,7-tetrahydro-6H-pyrazolo[3,4-c]pyridine-6-carbonitrile(4-(5-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; 3-(4-(3,5-difluoro-2-(trifluoromethyl)phenyl)piperidine-1-carbonyl)-N-methyl-1,4,5,7-tetrahydro-6H-pyrazolo[3,4-c]pyridine-6-carboxamide(6-(cy clopropylmethyl)-4,5,6,7-tetrahydro-1H-pyrazolo[3,4-c]pyridin-3-yl)(4-(3,4-difluoro-2-(trifluoromethyl)phenyl)piperidin-1-yl)methanone; methyl 3-(4-(3,5-difluoro-2-(trifluoromethyl)phenyl)piperidine-1-carbonyl)-1,4,5,7-tetrahydro-6H-pyrazolo[3,4-c]pyridine-6-carboxylate; (4-(3,5-difluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; (4-(3,4-difluoro-2-(trifluoromethyl)phenyl)piperidin-1-yl)(5-neopentyl-4,5,6,7-tetrahydro-1H-pyrazolo[4,3-c]pyridin-3-yl)methanone; (4-(2-chloro-5-fluorophenyl)piperidin-1-yl)(5-(methylsulfonyl)-4,5,6,7-tetrahydro-1H-pyrazolo[4,3-c]pyridin-3-yl)methanone; (4-(3,5-difluoro-2-(trifluoromethyl)phenyl)piperidin-1-yl)(6-(2,2,2-trifluoroethyl)-4,5,6,7-tetrahydro-1H-pyrazolo[3,4-c]pyridin-3-yl)methanone; 3-(4-(3,5-difluoro-2-(trifluoromethyl)phenyl)piperidine-1-carbonyl)-1,4,5,7-tetrahydro-6H-pyrazolo[3,4-c]pyridine-6-carbonitrile; (4-(3,5-difluoro-2-(trifluoromethyl)phenyl)piperidin-1-yl)(6-(oxetan-3-yl)-4,5,6,7-tetrahydro-1H-pyrazolo[3,4-c]pyridin-3-yl)methanone; 3-(4-(3,4-difluoro-2-(trifluoromethyl)phenyl)piperidine-1-carbonyl)-N-methyl-1,4,5,7-tetrahydro-6H-pyrazolo[3,4-c]pyridine-6-carboxamide; (4-(3,4-difluoro-2-(trifluoromethyl)phenyl)piperidin-1-yl)(6-neopentyl-4,5,6,7-tetrahydro-1H-pyrazolo[3,4-c]pyridin-3-yl)methanone; (4-(3,4-difluoro-2-(trifluoromethyl)phenyl)piperidin-1-yl)(6-(2,2,2-trifluoroethyl)-4,5,6,7-tetrahydro-1H-pyrazolo[3,4-c]pyridin-3-yl)methanone; methyl 3-(4-(2-chloro-3-fluorophenyl)piperidine-1-carbonyl)-1,4,5,7-tetrahydro-6H-pyrazolo[3,4-c]pyridine-6-carboxylate; 1-(3-(4-(3,5-difluoro-2-(trifluoromethyl)phenyl)piperidine-1-carbonyl)-1,4,5,7-tetrahydro-6H-pyrazolo[3,4-c]pyridin-6-yl)ethan-1-one; (6-(cyclopropylmethyl)-4,5,6,7-tetrahydro-1H-pyrazolo[3,4-c]pyridin-3-yl)(4-(3,5-difluoro-2-(trifluoromethyl)phenyl)piperidin-1-yl)methanone; (4-(3,4-difluoro-2-(trifluoromethyl)phenyl)piperidin-1-yl)(6-(2-methoxyethyl)-4,5,6,7-tetrahydro-1H-pyrazolo[3,4-c]pyridin-3-yl)methanone; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)-3-methylbutan-1-one; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)propan-1-one; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)-2-methylpropan-1-one; 3-(4-(3,4-difluoro-2-(trifluoromethyl)phenyl)piperidine-1-carbonyl)-1,4,5,7-tetrahydro-6H-pyrazolo[3,4-c]pyridine-6-carbonitrile; (4-(3,4-difluoro-2-(trifluoromethyl)phenyl)piperidin-1-yl)(6-(3,3,3-trifluoropropyl)-4,5,6,7-tetrahydro-1H-pyrazolo[3,4-c]pyridin-3-yl)methanone; (4-(3,4-difluoro-2-(trifluoromethyl)phenyl)piperidin-1-yl)(5-(2-methoxyethyl)-4,5,6,7-tetrahydro-1H-pyrazolo[4,3-c]pyridin-3-yl)methanone; (4-(3,4-difluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; 3-(4-(3,4-difluoro-2-(trifluoromethyl)phenyl)piperidine-1-carbonyl)-N-methyl-1,4,6,7-tetrahydro-5H-pyrazolo[4,3-c]pyridine-5-carboxamide; (4-(3,4-difluoro-2-(trifluoromethyl)phenyl)piperidin-1-yl)(6-(oxetan-3-yl)-4,5,6,7-tetrahydro-1H-pyrazolo[3,4-c]pyridin-3-yl)methanone; methyl 3-(4-(3,4-difluoro-2-(trifluoromethyl)phenyl)piperidine-1-carbonyl)-1,4,5,7-tetrahydro-6H-pyrazolo[3,4-c]pyridine-6-carboxylate; 2-(3-(4-(3,4-difluoro-2-(trifluoromethyl)phenyl)piperidine-1-carbonyl)-1,4,5,7-tetrahydro-6H-pyrazolo[3,4-c]pyridin-6-yl)acetic acid; (4-(3,4-difluoro-2-(trifluoromethyl)phenyl)piperidin-1-yl)(5-(oxetan-3-yl)-4,5,6,7-tetrahydro-1H-pyrazolo[4,3-c]pyridin-3-yl)methanone; (5-(cyclopropylmethyl)-4,5,6,7-tetrahydro-1H-pyrazolo[4,3-c]pyridin-3-yl)(4-(3,4-difluoro-2-(trifluoromethyl)phenyl)piperidin-1-yl)methanone; (4-(3,4-difluoro-2-(trifluoromethyl)phenyl)piperidin-1-yl)(5-ethyl-4,5,6,7-tetrahydro-1H-pyrazolo[4,3-c]pyridin-3-yl)methanone; 3-(4-(3-fluoro-2-(trifluoromethyl)phenyl)piperidine-1-carbonyl)-N-methyl-1,4,6,7-tetrahydro-5H-pyrazolo[4,3-c]pyridine-5-carboxamide; (4-(3,4-difluoro-2-(trifluoromethyl)phenyl)piperidin-1-yl)(6-methyl-4,5,6,7-tetrahydro-1H-pyrazolo[3,4-c]pyridin-3-yl)methanone; methyl 3-(4-(3,5-difluoro-2-(trifluoromethyl)phenyl)piperidine-1-carbonyl)-1,4,6,7-tetrahydro-5H-pyrazolo[4,3-c]pyridine-5-carboxylate; (4-(3,5-difluoro-2-(trifluoromethyl)phenyl)piperidin-1-yl)(5-ethyl-4,5,6,7-tetrahydro-1H-pyrazolo[4,3-c]pyridin-3-yl)methanone; (4-(3,5-difluoro-2-(trifluoromethyl)phenyl)piperidin-1-yl)(5-(2-methoxyethyl)-4,5,6,7-tetrahydro-TH-pyrazolo[4,3-c]pyridin-3-yl)methanone; 3-(4-(3,4-difluoro-2-(trifluoromethyl)phenyl)piperidine-1-carbonyl)-1,4,6,7-tetrahydro-5H-pyrazolo[4,3-c]pyridine-5-carbonitrile; methyl 3-(4-(3,4-difluoro-2-(trifluoromethyl)phenyl)piperidine-1-carbonyl)-1,4,6,7-tetrahydro-5H-pyrazolo[4,3-c]pyridine-5-carboxylate; (4-(3-fluoro-2-(trifluoromethyl)phenyl)piperidin-1-yl)(6-(3,3,3-trifluoropropyl)-4,5,6,7-tetrahydro-1H-pyrazolo[3,4-c]pyridin-3-yl)methanone; (6-ethyl-4,5,6,7-tetrahydro-1H-pyrazolo[3,4-c]pyridin-3-yl)(4-(3-fluoro-2-(trifluoromethyl)phenyl)piperidin-1-yl)methanone; (4-(3-fluoro-2-(trifluoromethyl)phenyl)piperidin-1-yl)(5-(oxetan-3-yl)-4,5,6,7-tetrahydro-1H-pyrazolo[4,3-c]pyridin-3-yl)methanone; (4-(3-fluoro-2-(trifluoromethyl)phenyl)piperidin-1-yl)(5-(2-methoxyethyl)-4,5,6,7-tetrahydro-1H-pyrazolo[4,3-c]pyridin-3-yl)methanone; (4-(3-fluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; (4-(2-chloro-5-fluorophenyl)piperidin-1-yl)(4,5,6,7-tetrahydro-1H-pyrazolo[3,4-c]pyridin-3-yl)methanone; imidazo[1,2-a]pyridin-2-yl(4-(2-(trifluoromethyl)phenyl)piperidin-1-yl)methanone; (4-(5-fluoro-2-(trifluoromethyl)phenyl)piperidin-1-yl)(4,5,6,7-tetrahydro-1H-pyrazolo[4,3-c]pyridin-3-yl)methanone; 1-(3-(4-(3,4-difluoro-2-(trifluoromethyl)phenyl)piperidine-1-carbonyl)-1,4,6,7-tetrahydro-5H-pyrazolo[4,3-c]pyridin-5-yl)ethan-1-one; (5-ethyl-4,5,6,7-tetrahydro-1H-pyrazolo[4,3-c]pyridin-3-yl)(4-(3-fluoro-2-(trifluoromethyl)phenyl)piperidin-1-yl)methanone; 3-(4-(3-fluoro-2-(trifluoromethyl)phenyl)piperidine-1-carbonyl)-N-methyl-1,4,5,7-tetrahydro-6H-pyrazolo[3,4-c]pyridine-6-carboxamide; (4-(3-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; (4-(3,5-difluoro-2-(trifluoromethyl)phenyl)piperidin-1-yl)(4,5,6,7-tetrahydro-1H-pyrazolo[4,3-c]pyridin-3-yl)methanone; (4-(3,5-difluoro-2-(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(4,5,6,7-tetrahydro-1H-pyrazolo[4,3-c]pyridin-3-yl)methanone; (4-(2-chloro-3-fluorophenyl)piperidin-1-yl)(4,5,6,7-tetrahydro-1H-pyrazolo[3,4-c]pyridin-3-yl)methanone; (4-(3,4-difluoro-2-(trifluoromethyl)phenyl)piperidin-1-yl)(4,5,6,7-tetrahydro-1H-pyrazolo[4,3-c]pyridin-3-yl)methanone; (4-(3,4-difluoro-2-(trifluoromethyl)phenyl)piperidin-1-yl)(4,5,6,7-tetrahydro-1H-pyrazolo[3,4-c]pyridin-3-yl)methanone; (4-(3,5-bis(trifluoromethyl)phenyl)piperidin-1-yl)(4,5,6,7-tetrahydro-1H-pyrazolo[4,3-c]pyridin-3-yl)methanone; (4-(3,5-bis(trifluoromethyl)phenyl)piperidin-1-yl)(4,5,6,7-tetrahydro-1H-pyrazolo[3,4-c]pyridin-3-yl)methanone; (4-(3-fluoro-2-(trifluoromethyl)phenyl)piperidin-1-yl)(4,5,6,7-tetrahydro-1H-pyrazolo[4,3-c]pyridin-3-yl)methanone; (4-(3-fluoro-2-(trifluoromethyl)phenyl)piperidin-1-yl)(4,5,6,7-tetrahydro-1H-pyrazolo[3,4-c]pyridin-3-yl)methanone; 1-(3-(4-(2-chloro-3-fluorophenyl)piperidine-1-carbonyl)-1,4,5,7-tetrahydro-6H-pyrazolo[3,4-c]pyridin-6-yl)ethan-1-one; 1-(3-(4-(3-fluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(5-fluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(3,5-bis(trifluoromethyl)phenyl)piperidine-1-carbonyl)-1,4,5,7-tetrahydro-6H-pyrazolo[3,4-c]pyridin-6-yl)ethan-1-one; 1-(3-(4-(4-fluoro-2-(trifluoromethyl)phenyl)piperidine-1-carbonyl)-1,4,5,7-tetrahydro-6H-pyrazolo[3,4-c]pyridin-6-yl)ethan-1-one; (4-(2-fluoro-6-(trifluoromethyl)phenyl)piperidin-1-yl)(4,5,6,7-tetrahydro-1H-pyrazolo[4,3-c]pyridin-3-yl)methanone; (4-(3-fluoro-2-(trifluoromethyl)phenyl)piperidin-1-yl)(5-(2,2,2-trifluoroethyl)-4,5,6,7-tetrahydro-1H-pyrazolo[4,3-c]pyridin-3-yl)methanone; (5-(cyclopropylmethyl)-4,5,6,7-tetrahydro-1H-pyrazolo[4,3-c]pyridin-3-yl)(4-(3-fluoro-2-(trifluoromethyl)phenyl)piperidin-1-yl)methanone; methyl 3-(4-(3-fluoro-2-(trifluoromethyl)phenyl)piperidine-1-carbonyl)-1,4,6,7-tetrahydro-5H-pyrazolo[4,3-c]pyridine-5-carboxylate; 3-(4-(3-fluoro-2-(trifluoromethyl)phenyl)piperidine-1-carbonyl)-1,4,6,7-tetrahydro-5H-pyrazolo[4,3-c]pyridine-5-carbonitrile; 1-(3-(4-(2-chloro-5-fluorophenyl)piperidine-1-carbonyl)-1,4,5,7-tetrahydro-6H-pyrazolo[3,4-c]pyridin-6-yl)ethan-1-one; (4-(2-fluoro-6-(trifluoromethyl)phenyl)piperidin-1-yl)(4,5,6,7-tetrahydro-1H-pyrazolo[3,4-c]pyridin-3-yl)methanone; tert-butyl 2-(3-(4-(3,4-difluoro-2-(trifluoromethyl)phenyl)piperidine-1-carbonyl)-1,4,5,7-tetrahydro-6H-pyrazolo[3,4-c]pyridin-6-yl)acetate; tert-butyl 3-(4-(3,5-difluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(3,5-difluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-fluoro-6-(trifluoromethyl)phenyl)piperidine-1-carbonyl)-1,4,6,7-tetrahydro-5H-pyrazolo[4,3-c]pyridine-5-carboxylate; tert-butyl 3-(4-(5-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(5-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-chloro-3-fluorophenyl)piperidine-1-carbonyl)-1,4,6,7-tetrahydro-5H-pyrazolo[4,3-c]pyridine-5-carboxylate; tert-butyl 3-(4-(2-fluoro-6-(trifluoromethyl)phenyl)piperidine-1-carbonyl)-1,4,5,7-tetrahydro-6H-pyrazolo[3,4-c]pyridine-6-carboxylatetert-butyl 3-(4-(3,5-bis(trifluoromethyl)phenyl)piperidine-1-carbonyl)-1,4,6,7-tetrahydro-5H-pyrazolo[4,3-c]pyridine-5-carboxylate; tert-butyl 3-(4-(3,5-bis(trifluoromethyl)phenyl)piperidine-1-carbonyl)-1,4,5,7-tetrahydro-6H-pyrazolo[3,4-c]pyridine-6-carboxylate; tert-butyl 3-(4-(2-chloro-5-fluorophenyl)piperidine-1-carbonyl)-1,4,6,7-tetrahydro-5H-pyrazolo[4,3-c]pyridine-5-carboxylate; tert-butyl 3-(4-(2-chloro-5-fluorophenyl)piperidine-1-carbonyl)-1,4,5,7-tetrahydro-6H-pyrazolo[3,4-c]pyridine-6-carboxylate; tert-butyl 3-(4-(4-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3,4-difluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(3,4-difluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(4-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(2-chloro-3-fluorophenyl)piperidine-1-carbonyl)-1,4,5,7-tetrahydro-6H-pyrazolo[3,4-c]pyridine-6-carboxylate; (6,6-dimethyl-1,4,6,7-tetrahydropyrano[4,3-c]pyrazol-3-yl)(4-(2-(trifluoromethyl)phenyl)piperidin-1-yl)methanone; (6,6-dioxido-1,4,5,7-tetrahydrothiopyrano[3,4-c]pyrazol-3-yl)(4-(2-(trifluoromethyl)phenyl)piperidin-1-yl)methanone; (1,4,6,7-tetrahydropyrano[4,3-c]pyrazol-3-yl)(4-(2-(trifluoromethyl)phenyl)piperidin-1-yl)methanone; (1,4,5,7-tetrahydropyrano[3,4-c]pyrazol-3-yl)(4-(2-(trifluoromethyl)phenyl)piperidin-1-yl)methanone; (1-methyl-4,5,6,7-tetrahydro-1H-pyrazolo[4,3-c]pyridin-3-yl)(4-(2-(trifluoromethyl)phenyl)piperidin-1-yl)methanone; (1-methyl-4,5,6,7-tetrahydro-1H-pyrazolo[3,4-c]pyridin-3-yl)(4-(2-(trifluoromethyl)phenyl)piperidin-1-yl)methanone; 1-ethyl-N,N-dimethyl-3-(4-(2-(trifluoromethyl)phenyl)piperidine-1-carbonyl)-1,4,5,7-tetrahydro-6H-pyrazolo[3,4-c]pyridine-6-carboxamide; (5-(2,2,2-trifluoroethyl)-4,5,6,7-tetrahydro-1H-pyrazolo[4,3-c]pyridin-3-yl)(4-(2-(trifluoromethyl)phenyl)piperidin-1-yl)methanone; (6-(2,2,2-trifluoroethyl)-4,5,6,7-tetrahydro-1H-pyrazolo[3,4-c]pyridin-3-yl)(4-(2-(trifluoromethyl)phenyl)piperidin-1-yl)methanone; (5-chloro-1H-indazol-3-yl)(4-(2-(trifluoromethyl)phenyl)piperidin-1-yl)methanone; (1H-pyrazolo[3,4-b]pyridin-3-yl)(4-(2-(trifluoromethyl)phenyl)piperidin-1-yl)methanone; (6-chloro-1H-indazol-3-yl)(4-(2-(trifluoromethyl)phenyl)piperidin-1-yl)methanone; (5-(methylsulfonyl)-4,5,6,7-tetrahydro-1H-pyrazolo[4,3-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,6,7-tetrahydro-5H-pyrazolo[4,3-c]pyridin-5-yl)ethan-1-one; (4,5,6,7-tetrahydro-1H-pyrazolo[4,3-c]pyridin-3-yl)(4-(2-(trifluoromethyl)phenyl)piperidin-1-yl)methanone; (6-fluoro-1-(oxetan-3-yl)-1H-indazol-3-yl)(4-(2-(trifluoromethyl)phenyl)piperidin-1-yl)methanone; (1-ethyl-6-fluoro-1H-indazol-3-yl)(4-(2-(trifluoromethyl)phenyl)piperidin-1-yl)methanone; (6-fluoro-1-isopropyl-1H-indazol-3-yl)(4-(2-(trifluoromethyl)phenyl)piperidin-1-yl)methanone; 1-(3-(4-fluoro-4-(2-(trifluoromethyl)phenyl)piperidine-1-carbonyl)-1,4,6,7-tetrahydro-5H-pyrazolo[4,3-c]pyridin-5-yl)ethan-1-one; (5-fluoro-1-methyl-1H-indazol-3-yl)(4-(2-(trifluoromethyl)phenyl)piperidin-1-yl)methanone; (6-fluoro-1H-indazol-3-yl)(4-(2-(trifluoromethyl)phenyl)piperidin-1-yl)methanone; (6-methyl-4,5,6,7-tetrahydro-1H-pyrazolo[3,4-c]pyridin-3-yl)(4-(2-(trifluoromethyl)phenyl)piperidin-1-yl)methanone; (6-(methylsulfonyl)-4,5,6,7-tetrahydro-1H-pyrazolo[3,4-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,5,7-tetrahydro-6H-pyrazolo[3,4-c]pyridin-6-yl)ethan-1-one; (5-fluoro-1H-indazol-3-yl)(4-(2-(trifluoromethyl)phenyl)piperidin-1-yl)methanone; (5-((chloromethyl)sulfonyl)-4,5,6,7-tetrahydro-1H-pyrazolo[4,3-c]pyridin-3-yl)(4-(2-(trifluoromethyl)phenyl)piperidin-1-yl)methanone; (5-(2-methoxyethyl)-4,5,6,7-tetrahydro-1H-pyrazolo[4,3-c]pyridin-3-yl)(4-(2-(trifluoromethyl)phenyl)piperidin-1-yl)methanone; (4-fluoro-4-(2-(trifluoromethyl)phenyl)piperidin-1-yl)(4,5,6,7-tetrahydro-1H-pyrazolo[4,3-c]pyridin-3-yl)methanone; 1-(3-(4-(4-fluoro-2-(trifluoromethyl)phenyl)piperidine-1-carbonyl)-1,4,5,7-tetrahydro-6H-pyrazolo[3,4-c]pyricdin-6-yl)ethan-1-one; (1-ethyl-5-fluoro-1H-indazol-3-yl)(4-(2-(trifluoromethyl)phenyl)piperidin-1-yl)methanone; (6-fluoro-1-methyl-1H-indazol-3-yl)(4-(2-(trifluoromethyl)phenyl)piperidin-1-yl)methanone; 3-(4-(2-(trifluoromethyl)phenyl)piperidine-1-carbonyl)-1,4,5,7-tetrahydro-6H-pyrazolo[4,3-c]pyridin-6-one; 3-(4-(2-(trifluoromethyl)phenyl)piperidine-1-carbonyl)-1,4,6,7-tetrahydro-5H-pyrazolo[3,4-c]pyridin-5-one; 6-methyl-3-(4-(2-(trifluoromethyl)phenyl)piperidine-1-carbonyl)-1,4,6,7-tetrahydro-5H-pyrazolo[3,4-c]pyridin-5-one; 5-methyl-3-(4-(2-(trifluoromethyl)phenyl)piperidine-1-carbonyl)-1,4,5,7-tetrahydro-6H-pyrazolo[4,3-c]pyridin-6-one; (5,5-dioxido-1,4,6,7-tetrahydrothiopyrano[4,3-c]pyrazol-3-yl)(4-(2-(trifluoromethyl)phenyl)piperidin-1-yl)methanone; (1-methyl-5-(methylsulfonyl)-4,5,6,7-tetrahydro-1H-pyrazolo[4,3-c]pyridin-3-yl)(4-(2-(trifluoromethyl)phenyl)piperidin-1-yl)methanone; (1-methyl-6-(methylsulfonyl)-4,5,6,7-tetrahydro-1H-pyrazolo[3,4-c]pyridin-3-yl)(4-(2-(trifluoromethyl)phenyl)piperidin-1-yl)methanone; 1-(1-ethyl-3-(4-(2-(trifluoromethyl)phenyl)piperidine-1-carbonyl)-1,4,5,7-tetrahydro-6H-pyrazolo[3,4-c]pyridin-6-yl)ethan-1-one; (5-(methoxymethyl)-4,5,6,7-tetrahydro-1H-pyrazolo[4,3-c]pyridin-3-yl)(4-(2-(trifluoromethyl)phenyl)piperidin-1-yl)methanone; (6-(methoxymethyl)-4,5,6,7-tetrahydro-1H-pyrazolo[3,4-c]pyridin-3-yl)(4-(2-(trifluoromethyl)phenyl)piperidin-1-yl)methanone; (5-methoxy-1H-indazol-3-yl)(4-(2-(trifluoromethyl)phenyl)piperidin-1-yl)methanone; (5-(oxetan-3-yl)-4,5,6,7-tetrahydro-1H-pyrazolo[4,3-c]pyridin-3-yl)(4-(2-(trifluoromethyl)phenyl)piperidin-1-yl)methanone; (5-isobutyl-4,5,6,7-tetrahydro-1H-pyrazolo[4,3-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,6,7-tetrahydro-5H-pyrazolo[4,3-c]pyridin-5-yl)propan-1-one; (5-ethyl-4,5,6,7-tetrahydro-1H-pyrazolo[4,3-c]pyridin-3-yl)(4-(2-(trifluoromethyl)phenyl)piperidin-1-yl)methanone; 3-methyl-1-(3-(4-(2-(trifluoromethyl)phenyl)piperidine-1-carbonyl)-1,4,6,7-tetrahydro-5H-pyrazolo[4,3-c]pyridin-5-yl)butan-1-one; 2-methyl-1-(3-(4-(2-(trifluoromethyl)phenyl)piperidine-1-carbonyl)-1,4,6,7-tetrahydro-5H-pyrazolo[4,3-c]pyridin-5-yl)propan-1-one; 2,2-dimethyl-1-(3-(4-(2-(trifluoromethyl)phenyl)piperidine-1-carbonyl)-1,4,6,7-tetrahydro-5H-pyrazolo[4,3-c]pyridin-5-yl)propan-1-one; (5-(isopropylsulfonyl)-4,5,6,7-tetrahydro-1H-pyrazolo[4,3-c]pyridin-3-yl)(4-(2-(trifluoromethyl)phenyl)piperidin-1-yl)methanone; (5-(isobutylsulfonyl)-4,5,6,7-tetrahydro-1H-pyrazolo[4,3-c]pyridin-3-yl)(4-(2-(trifluoromethyl)phenyl)piperidin-1-yl)methanone; (5-(ethylsulfonyl)-4,5,6,7-tetrahydro-1H-pyrazolo[4,3-c]pyridin-3-yl)(4-(2-(trifluoromethyl)phenyl)piperidin-1-yl)methanone; 3-(4-(2-(trifluoromethyl)phenyl)piperidine-1-carbonyl)-1H-indazole-5-carbonitrile; (7-chloro-1H-indazol-3-yl)(4-(2-(trifluoromethyl)phenyl)piperidin-1-yl)methanone; (5,6-difluoro-1H-indazol-3-yl)(4-(2-(trifluoromethyl)phenyl)piperidin-1-yl)methanone; (6-(2-methoxyethyl)-4,5,6,7-tetrahydro-1H-pyrazolo[3,4-c]pyridin-3-yl)(4-(2-(trifluoromethyl)phenyl)piperidin-1-yl)methanone; 3,3,3-trifluoro-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (5-(tert-butyl)-4,5,6,7-tetrahydro-1H-pyrazolo[4,3-c]pyridin-3-yl)(4-(2-(trifluoromethyl)phenyl)piperidin-1-yl)methanone; (5-isopropyl-4,5,6,7-tetrahydro-TH-pyrazolo[4,3-c]pyridin-3-yl)(4-(2-(trifluoromethyl)phenyl)piperidin-1-yl)methanone; N-methyl-3-(4-(2-(trifluoromethyl)phenyl)piperidine-1-carbonyl)-1,4,6,7-tetrahydro-5H-pyrazolo[4,3-c]pyridine-5-carboxamide; N-methyl-3-(4-(2-(trifluoromethyl)phenyl)piperidine-1-carbonyl)-1,4,5,7-tetrahydro-6H-pyrazolo[3,4-c]pyridine-6-carboxamide; (5-bromo-1H-indazol-3-yl)(4-(2-(trifluoromethyl)phenyl)piperidin-1-yl)methanone; tert-butyl 3-(4-(2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-(trifluoromethyl)phenyl)piperidine-1-carbonyl)-1,4,6,7-tetrahydro-5H-pyrazolo[4,3-c]pyridine-5-carboxylate; (5-fluoro-1-isopropyl-1H-indazol-3-yl)(4-(2-(trifluoromethyl)phenyl)piperidin-1-yl)methanone; (7-fluoro-1H-indazol-3-yl)(4-(2-(trifluoromethyl)phenyl)piperidin-1-yl)methanone; (1H-pyrazolo[4,3-c]pyridin-3-yl)(4-(2-(trifluoromethyl)phenyl)piperidin-1-yl)methanone; (1H-pyrazolo[3,4-c]pyridin-3-yl)(4-(2-(trifluoromethyl)phenyl)piperidin-1-yl)methanone; (1H-pyrazolo[4,3-b]pyridin-3-yl)(4-(2-(trifluoromethyl)phenyl)piperidin-1-yl)methanone; (6-methoxy-1H-indazol-3-yl)(4-(2-(trifluoromethyl)phenyl)piperidin-1-yl)methanone; (5-fluoro-1-(oxetan-3-yl)-1H-indazol-3-yl)(4-(2-(trifluoromethyl)phenyl)piperidin-1-yl)methanone; (1-ethyl-5-(methylsulfonyl)-4,5,6,7-tetrahydro-1H-pyrazolo[4,3-c]pyridin-3-yl)(4-(2-(trifluoromethyl)phenyl)piperidin-1-yl)methanone; (1-ethyl-6-(methylsulfonyl)-4,5,6,7-tetrahydro-1H-pyrazolo[3,4-c]pyridin-3-yl)(4-(2-(trifluoromethyl)phenyl)piperidin-1-yl)methanone; 1-(1-methyl-3-(4-(2-(trifluoromethyl)phenyl)piperidine-1-carbonyl)-1,4,6,7-tetrahydro-5H-pyrazolo[4,3-c]pyridin-5-yl)ethan-1-one; 1-(1-methyl-3-(4-(2-(trifluoromethyl)phenyl)piperidine-1-carbonyl)-1,4,5,7-tetrahydro-6H-pyrazolo[3,4-c]pyridin-6-yl)ethan-1-one; N,N-dimethyl-3-(4-(2-(trifluoromethyl)phenyl)piperidine-1-carbonyl)-1,4,6,7-tetrahydro-5H-pyrazolo[4,3-c]pyridine-5-carboxamide; N,N-dimethyl-3-(4-(2-(trifluoromethyl)phenyl)piperidine-1-carbonyl)-1,4,5,7-tetrahydro-6H-pyrazolo[3,4-c]pyridine-6-carboxamide; (1-methyl-5,5-dioxido-1,4,6,7-tetrahydrothiopyrano[4,3-c]pyrazol-3-yl)(4-(2-(trifluoromethyl)phenyl)piperidin-1-yl)methanone; (4-(2-(trifluoromethyl)phenyl)piperidin-1-yl)(1,6,6-trimethyl-1,4,6,7-tetrahydropyrano[4,3-c]pyrazol-3-yl)methanone; (1-methyl-1,4,6,7-tetrahydropyrano[4,3-c]pyrazol-3-yl)(4-(2-(trifluoromethyl)phenyl)piperidin-1-yl)methanone; 2-methyl-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (6-(isopropylsulfonyl)-4,5,6,7-tetrahydro-1H-pyrazolo[3,4-c]pyridin-3-yl)(4-(2-(trifluoromethyl)phenyl)piperidin-1-yl)methanone; (6-(ethylsulfonyl)-4,5,6,7-tetrahydro-TH-pyrazolo[3,4-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,5,7-tetrahydro-6H-pyrazolo[3,4-c]pyridin-6-yl)propan-1-one; 2-methoxy-1-(3-(4-(2-(trifluoromethyl)phenyl)piperidine-1-carbonyl)-1,4,6,7-tetrahydro-5H-pyrazolo[4,3-c]pyridin-5-yl)ethan-1-one; 3,3,3-trifluoro-1-(3-(4-(2-(trifluoromethyl)phenyl)piperidine-1-carbonyl)-1,4,6,7-tetrahydro-5H-pyrazolo[4,3-c]pyridin-5-yl)propan-1-one; (1H-indazol-3-yl)(4-(2-(trifluoromethyl)phenyl)piperidin-1-yl)methanone; (1-methyl-1H-indazol-3-yl)(4-(2-(trifluoromethyl)phenyl)piperidin-1-yl)methanone; (6-(oxetan-3-yl)-4,5,6,7-tetrahydro-1H-pyrazolo[3,4-c]pyridin-3-yl)(4-(2-(trifluoromethyl)phenyl)piperidin-1-yl)methanone; (6-(tert-butylsulfonyl)-4,5,6,7-tetrahydro-1H-pyrazolo[3,4-c]pyridin-3-yl)(4-(2-(trifluoromethyl)phenyl)piperidin-1-yl)methanone; 2,2-dimethyl-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (6-(tert-butyl)-4,5,6,7-tetrahydro-1H-pyrazolo[3,4-c]pyridin-3-yl)(4-(2-(trifluoromethyl)phenyl)piperidin-1-yl)methanone; (6-(isobutylsulfonyl)-4,5,6,7-tetrahydro-1H-pyrazolo[3,4-c]pyridin-3-yl)(4-(2-(trifluoromethyl)phenyl)piperidin-1-yl)methanone; 3-methyl-1-(3-(4-(2-(trifluoromethyl)phenyl)piperidine-1-carbonyl)-1,4,5,7-tetrahydro-6H-pyrazolo[3,4-c]pyridin-6-yl)butan-1-one; (6-isobutyl-4,5,6,7-tetrahydro-1H-pyrazolo[3,4-c]pyridin-3-yl)(4-(2-(trifluoromethyl)phenyl)piperidin-1-yl)methanone; (6-isopropyl-4,5,6,7-tetrahydro-1H-pyrazolo[3,4-c]pyridin-3-yl)(4-(2-(trifluoromethyl)phenyl)piperidin-1-yl)methanone; (6-ethyl-4,5,6,7-tetrahydro-1H-pyrazolo[3,4-c]pyridin-3-yl)(4-(2-(trifluoromethyl)phenyl)piperidin-1-yl)methanone; (5-(tert-butylsulfonyl)-4,5,6,7-tetrahydro-1H-pyrazolo[4,3-c]pyridin-3-yl)(4-(2-(trifluoromethyl)phenyl)piperidin-1-yl)methanone; tert-butyl 3-(4-fluoro-4-(2-(trifluoromethyl)phenyl)piperidine-1-carbonyl)-1,4,6,7-tetrahydro-5H-pyrazolo[4,3-c]pyridine-5-carboxylate; (4-hydroxy-4-(2-(trifluoromethyl)phenyl)piperidin-1-yl)(1-methyl-1H-indazol-3-yl)methanone; 1-(3-(4-hydroxy-4-(2-(trifluoromethyl)phenyl)piperidine-1-carbonyl)-1,4,6,7-tetrahydro-5H-pyrazolo[4,3-c]pyridin-5-yl)ethan-1-one; 3-(4-(3,4-difluoro-2-(trifluoromethyl)phenyl)piperidine-1-carbonyl)-N-methyl-4,6-dihydropyrrolo[3,4-c]pyrazole-5(1H)-carboxamide; (4-(3,4-difluoro-2-(trifluoromethyl)phenyl)piperidin-1-yl)(5-neopentyl-1,4,5,6-tetrahydropyrrolo[3,4-c]pyrazol-3-yl)methanone; (4-(3,4-difluoro-2-(trifluoromethyl)phenyl)piperidin-1-yl)(5-(oxetan-3-yl)-1,4,5,6-tetrahydropyrrolo[3,4-c]pyrazol-3-yl)methanone; (5-(cyclopropylmethyl)-1,4,5,6-tetrahydropyrrolo[3,4-c]pyrazol-3-yl)(4-(3,4-difluoro-2-(trifluoromethyl)phenyl)piperidin-1-yl)methanone; (4-(3,4-difluoro-2-(trifluoromethyl)phenyl)piperidin-1-yl)(5-ethyl-1,4,5,6-tetrahydropyrrolo[3,4-c]pyrazol-3-yl)methanone; 1-(3-(4-(3,4-difluoro-2-(trifluoromethyl)phenyl)piperidine-1-carbonyl)-4,6-dihydropyrrolo[3,4-c]pyrazol-5(1H)-yl)-3-methylbutan-1-one; 1-(3-(4-(3,4-difluoro-2-(trifluoromethyl)phenyl)piperidine-1-carbonyl)-4,6-dihydropyrrolo[3,4-c]pyrazol-5(1H)-yl)-2-methylpropan-1-one; (4-(3,4-difluoro-2-(trifluoromethyl)phenyl)piperidin-1-yl)(5-picolinoyl-1,4,5,6-tetrahydropyrrolo[3,4-c]pyrazol-3-yl)methanone; 3-(4-(3,4-difluoro-2-(trifluoromethyl)phenyl)piperidine-1-carbonyl)-4,6-dihydropyrrolo[3,4-c]pyrazole-5(1H)-carbonitrile; (4-(3,4-difluoro-2-(trifluoromethyl)phenyl)piperidin-1-yl)(5-(2-methoxyethyl)-1,4,5,6-tetrahydropyrrolo[3,4-c]pyrazol-3-yl)methanone; (4-(3,4-difluoro-2-(trifluoromethyl)phenyl)piperidin-1-yl)(5-(3,3,3-trifluoropropyl)-1,4,5,6-tetrahydropyrrolo[3,4-c]pyrazol-3-yl)methanone; (5-benzoyl-1,4,5,6-tetrahydropyrrolo[3,4-c]pyrazol-3-yl)(4-(3,4-difluoro-2-(trifluoromethyl)phenyl)piperidin-1-yl)methanone; methyl 3-(4-(3,4-difluoro-2-(trifluoromethyl)phenyl)piperidine-1-carbonyl)-4,6-dihydropyrrolo[3,4-c]pyrazole-5(1H)-carboxylate; (4-(3,4-difluoro-2-(trifluoromethyl)phenyl)piperidin-1-yl)(1,4,5,6-tetrahydropyrrolo[3,4-c]pyrazol-3-yl)methanone; (4-(3,4-difluoro-2-(trifluoromethyl)phenyl)piperidin-1-yl)(5-(2,2,2-trifluoroethyl)-1,4,5,6-tetrahydropyrrolo[3,4-c]pyrazol-3-yl)methanone; (4-(3,4-difluoro-2-(trifluoromethyl)phenyl)piperidin-1-yl)(5-(pyrrolidine-1-carbonyl)-1,4,5,6-tetrahydropyrrolo[3,4-c]pyrazol-3-yl)methanone; (4-(3,4-difluoro-2-(trifluoromethyl)phenyl)piperidin-1-yl)(5-isonicotinoyl-1,4,5,6-tetrahydropyrrolo[3,4-c]pyrazol-3-yl)methanone; (4-(3,4-difluoro-2-(trifluoromethyl)phenyl)piperidin-1-yl)(5-nicotinoyl-1,4,5,6-tetrahydropyrrolo[3,4-c]pyrazol-3-yl)methanone; (4-(3,4-difluoro-2-(trifluoromethyl)phenyl)piperidin-1-yl)(5-(piperidine-1-carbonyl)-1,4,5,6-tetrahydropyrrolo[3,4-c]pyrazol-3-yl)methanone; (4-(3,4-difluoro-2-(trifluoromethyl)phenyl)piperidin-1-yl)(5-(piperazine-1-carbonyl)-1,4,5,6-tetrahydropyrrolo[3,4-c]pyrazol-3-yl)methanone; 1-(3-(4-(3,4-difluoro-2-(trifluoromethyl)phenyl)piperidine-1-carbonyl)-4,6-dihydropyrrolo[3,4-c]pyrazol-5(1H)-yl)propan-1-one; (5,5-dioxido-4,6-dihydro-1H-thieno[3,4-c]pyrazol-3-yl)(4-(2-(trifluoromethyl)phenyl)piperidin-1-yl)methanone; (4,6-dihydro-1H-furo[3,4-c]pyrazol-3-yl)(4-(2-(trifluoromethyl)phenyl)piperidin-1-yl)methanone; (1-ethyl-5-(methylsulfonyl)-1,4,5,6-tetrahydropyrrolo[3,4-c]pyrazol-3-yl)(4-(2-(trifluoromethyl)phenyl)piperidin-1-yl)methanone; (1-methyl-5-(methylsulfonyl)-1,4,5,6-tetrahydropyrrolo[3,4-c]pyrazol-3-yl)(4-(2-(trifluoromethyl)phenyl)piperidin-1-yl)methanone; (1-methyl-1,4,5,6-tetrahydropyrrolo[3,4-c]pyrazol-3-yl)(4-(2-(trifluoromethyl)phenyl)piperidin-1-yl)methanone; 2-methoxy-1-(3-(4-(2-(trifluoromethyl)phenyl)piperidine-1-carbonyl)-4,6-dihydropyrrolo[3,4-c]pyrazol-5(1H)-yl)ethan-1-one; (5-(2-methoxyethyl)-1,4,5,6-tetrahydropyrrolo[3,4-c]pyrazol-3-yl)(4-(2-(trifluoromethyl)phenyl)piperidin-1-yl)methanone; 3,3,3-trifluoro-1-(3-(4-(2-(trifluoromethyl)phenyl)piperidine-1-carbonyl)-4,6-dihydropyrrolo[3,4-c]pyrazol-5(1H)-yl)propan-1-one; (5-(oxetan-3-yl)-1,4,5,6-tetrahydropyrrolo[3,4-c]pyrazol-3-yl)(4-(2-(trifluoromethyl)phenyl)piperidin-1-yl)methanone; (5-(isobutylsulfonyl)-1,4,5,6-tetrahydropyrrolo[3,4-c]pyrazol-3-yl)(4-(2-(trifluoromethyl)phenyl)piperidin-1-yl)methanone; (5-(isopropylsulfonyl)-1,4,5,6-tetrahydropyrrolo[3,4-c]pyrazol-3-yl)(4-(2-(trifluoromethyl)phenyl)piperidin-1-yl)methanone; (5-(ethylsulfonyl)-1,4,5,6-tetrahydropyrrolo[3,4-c]pyrazol-3-yl)(4-(2-(trifluoromethyl)phenyl)piperidin-1-yl)methanone; tert-butyl 3-(4-(2-(trifluoromethyl)phenyl)piperidine-1-carbonyl)-4,6-dihydropyrrolo[3,4-c]pyrazole-5(1H)-carboxylate; (5-methyl-1,4,5,6-tetrahydropyrrolo[3,4-c]pyrazol-3-yl)(4-(2-(trifluoromethyl)phenyl)piperidin-1-yl)methanone; (5-(methylsulfonyl)-1,4,5,6-tetrahydropyrrolo[3,4-c]pyrazol-3-yl)(4-(2-(trifluoromethyl)phenyl)piperidin-1-yl)methanone; 1-(3-(4-(2-(trifluoromethyl)phenyl)piperidine-1-carbonyl)-4,6-dihydropyrrolo[3,4-c]pyrazol-5(1H)-yl)ethan-1-one; (5-(tert-butylsulfonyl)-1,4,5,6-tetrahydropyrrolo[3,4-c]pyrazol-3-yl)(4-(2-(trifluoromethyl)phenyl)piperidin-1-yl)methanone; (5-(tert-butyl)-1,4,5,6-tetrahydropyrrolo[3,4-c]pyrazol-3-yl)(4-(2-(trifluoromethyl)phenyl)piperidin-1-yl)methanone; (5-isobutyl-1,4,5,6-tetrahydropyrrolo[3,4-c]pyrazol-3-yl)(4-(2-(trifluoromethyl)phenyl)piperidin-1-yl)methanone; (5-isopropyl-1,4,5,6-tetrahydropyrrolo[3,4-c]pyrazol-3-yl)(4-(2-(trifluoromethyl)phenyl)piperidin-1-yl)methanone; (5-ethyl-1,4,5,6-tetrahydropyrrolo[3,4-c]pyrazol-3-yl)(4-(2-(trifluoromethyl)phenyl)piperidin-1-yl)methanone; N-methyl-3-(4-(2-(trifluoromethyl)phenyl)piperidine-1-carbonyl)-4,6-dihydropyrrolo[3,4-c]pyrazole-5(1H)-carboxamide; 1-(1-methyl-3-(4-(2-(trifluoromethyl)phenyl)piperidine-1-carbonyl)-4,6-dihydropyrrolo[3,4-c]pyrazol-5(1H)-yl)ethan-1-one; 2,2-dimethyl-1-(3-(4-(2-(trifluoromethyl)phenyl)piperidine-1-carbonyl)-4,6-dihydropyrrolo[3,4-c]pyrazol-5(1H)-yl)propan-1-one; 3-methyl-1-(3-(4-(2-(trifluoromethyl)phenyl)piperidine-1-carbonyl)-4,6-dihydropyrrolo[3,4-c]pyrazol-5(1H)-yl)butan-1-one; 2-methyl-1-(3-(4-(2-(trifluoromethyl)phenyl)piperidine-1-carbonyl)-4,6-dihydropyrrolo[3,4-c]pyrazol-5(1H)-yl)propan-1-one; 1-(3-(4-(2-(trifluoromethyl)phenyl)piperidine-1-carbonyl)-4,6-dihydropyrrolo[3,4-c]pyrazol-5(1H)-yl)propan-1-one; N,N-dimethyl-3-(4-(2-(trifluoromethyl)phenyl)piperidine-1-carbonyl)-4,6-dihydropyrrolo[3,4-c]pyrazole-5(1H)-carboxamide; (5-(2,2,2-trifluoroethyl)-1,4,5,6-tetrahydropyrrolo[3,4-c]pyrazol-3-yl)(4-(2-(trifluoromethyl)phenyl)piperidin-1-yl)methanone; (5-(methoxymethyl)-1,4,5,6-tetrahydropyrrolo[3,4-c]pyrazol-3-yl)(4-(2-(trifluoromethyl)phenyl)piperidin-1-yl)methanone; (1,4,5,6-tetrahydropyrrolo[3,4-c]pyrazol-3-yl)(4-(2-(trifluoromethyl)phenyl)piperidin-1-yl)methanone; tert-butyl 4-(3-(4-(3,4-difluoro-2-(trifluoromethyl)phenyl)piperidine-1-carbonyl)-1,4,5,6-tetrahydropyrrolo[3,4-c]pyrazole-5-carbonyl)piperazine-1-carboxylate; tert-butyl 3-(4-(3,4-difluoro-2-(trifluoromethyl)phenyl)piperidine-1-carbonyl)-4,6-dihydropyrrolo[3,4-c]pyrazole-5(1H)-carboxylate; (5,5-dioxido-4,6,7,8-tetrahydro-1H-thiepino[4,3-c]pyrazol-3-yl)(4-(2-(trifluoromethyl)phenyl)piperidin-1-yl)methanone; (4,6,7,8-tetrahydro-1H-oxepino[4,3-c]pyrazol-3-yl)(4-(2-(trifluoromethyl)phenyl)piperidin-1-yl)methanone; N-methyl-3-(4-(2-(trifluoromethyl)phenyl)piperidine-1-carbonyl)-4,6,7,8-tetrahydropyrazolo[4,3-c]azepine-5(1H)-carboxamide; (5-(2,2,2-trifluoroethyl)-1,4,5,6,7,8-hexahydropyrazolo[4,3-c]azepin-3-yl)(4-(2-(trifluoromethyl)phenyl)piperidin-1-yl)methanone; (5-(tert-butylsulfonyl)-1,4,5,6,7,8-hexahydropyrazolo[4,3-c]azepin-3-yl)(4-(2-(trifluoromethyl)phenyl)piperidin-1-yl)methanone; 2,2-dimethyl-1-(3-(4-(2-(trifluoromethyl)phenyl)piperidine-1-carbonyl)-4,6,7,8-tetrahydropyrazolo[4,3-c]azepin-5(1H)-yl)propan-1-one; (5-(tert-butyl)-1,4,5,6,7,8-hexahydropyrazolo[4,3-c]azepin-3-yl)(4-(2-(trifluoromethyl)phenyl)piperidin-1-yl)methanone; (5-(isobutylsulfonyl)-1,4,5,6,7,8-hexahydropyrazolo[4,3-c]azepin-3-yl)(4-(2-(trifluoromethyl)phenyl)piperidin-1-yl)methanone; 3-methyl-1-(3-(4-(2-(trifluoromethyl)phenyl)piperidine-1-carbonyl)-4,6,7,8-tetrahydropyrazolo[4,3-c]azepin-5(1H)-yl)butan-1-one; (5-isobutyl-1,4,5,6,7,8-hexahydropyrazolo[4,3-c]azepin-3-yl)(4-(2-(trifluoromethyl)phenyl)piperidin-1-yl)methanone; (5-(isopropylsulfonyl)-1,4,5,6,7,8-hexahydropyrazolo[4,3-c]azepin-3-yl)(4-(2-(trifluoromethyl)phenyl)piperidin-1-yl)methanone; 2-methyl-1-(3-(4-(2-(trifluoromethyl)phenyl)piperidine-1-carbonyl)-4,6,7,8-tetrahydropyrazolo[4,3-c]azepin-5(1H)-yl)propan-1-one; (1-ethyl-5-(methylsulfonyl)-1,4,5,6,7,8-hexahydropyrazolo[4,3-c]azepin-3-yl)(4-(2-(trifluoromethyl)phenyl)piperidin-1-yl)methanone; 1-(1-methyl-3-(4-(2-(trifluoromethyl)phenyl)piperidine-1-carbonyl)-4,6,7,8-tetrahydropyrazolo[4,3-c]azepin-5(1H)-yl)ethan-1-one; (5-(methoxymethyl)-1,4,5,6,7,8-hexahydropyrazolo[4,3-c]azepin-3-yl)(4-(2-(trifluoromethyl)phenyl)piperidin-1-yl)methanone; (5-methyl-1,4,5,6,7,8-hexahydropyrazolo[4,3-c]azepin-3-yl)(4-(2-(trifluoromethyl)phenyl)piperidin-1-yl)methanone; (5-isopropyl-1,4,5,6,7,8-hexahydropyrazolo[4,3-c]azepin-3-yl)(4-(2-(trifluoromethyl)phenyl)piperidin-1-yl)methanone; (5-(ethylsulfonyl)-1,4,5,6,7,8-hexahydropyrazolo[4,3-c]azepin-3-yl)(4-(2-(trifluoromethyl)phenyl)piperidin-1-yl)methanone; 1-(3-(4-(2-(trifluoromethyl)phenyl)piperidine-1-carbonyl)-4,6,7,8-tetrahydropyrazolo[4,3-c]azepin-5(1H)-yl)propan-1-one; (5-ethyl-1,4,5,6,7,8-hexahydropyrazolo[4,3-c]azepin-3-yl)(4-(2-(trifluoromethyl)phenyl)piperidin-1-yl)methanone; (5-(methylsulfonyl)-1,4,5,6,7,8-hexahydropyrazolo[4,3-c]azepin-3-yl)(4-(2-(trifluoromethyl)phenyl)piperidin-1-yl)methanone; 1-(3-(4-(2-(trifluoromethyl)phenyl)piperidine-1-carbonyl)-4,6,7,8-tetrahydropyrazolo[4,3-c]azepin-5(1H)-yl)ethan-1-one; (1,4,5,6,7,8-hexahydropyrazolo[4,3-c]azepin-3-yl)(4-(2-(trifluoromethyl)phenyl)piperidin-1-yl)methanone; (1-methyl-5-(methylsulfonyl)-1,4,5,6,7,8-hexahydropyrazolo[4,3-c]azepin-3-yl)(4-(2-(trifluoromethyl)phenyl)piperidin-1-yl)methanone; (1-methyl-1,4,5,6,7,8-hexahydropyrazolo[4,3-c]azepin-3-yl)(4-(2-(trifluoromethyl)phenyl)piperidin-1-yl)methanone; N,N-dimethyl-3-(4-(2-(trifluoromethyl)phenyl)piperidine-1-carbonyl)-4,6,7,8-tetrahydropyrazolo[4,3-c]azepine-5(1H)-carboxamide; (5-(2-methoxyethyl)-1,4,5,6,7,8-hexahydropyrazolo[4,3-c]azepin-3-yl)(4-(2-(trifluoromethyl)phenyl)piperidin-1-yl)methanone; 2-methoxy-1-(3-(4-(2-(trifluoromethyl)phenyl)piperidine-1-carbonyl)-4,6,7,8-tetrahydropyrazolo[4,3-c]azepin-5(1H)-yl)ethan-1-one; 3,3,3-trifluoro-1-(3-(4-(2-(trifluoromethyl)phenyl)piperidine-1-carbonyl)-4,6,7,8-tetrahydropyrazolo[4,3-c]azepin-5(1H)-yl)propan-1-one; (5-(oxetan-3-yl)-1,4,5,6,7,8-hexahydropyrazolo[4,3-c]azepin-3-yl)(4-(2-(trifluoromethyl)phenyl)piperidin-1-yl)methanone; tert-butyl 3-(4-(2-(trifluoromethyl)phenyl)piperidine-1-carbonyl)-4,6,7,8-tetrahydropyrazolo[4,3-c]azepine-5(1H)-carboxylate; (6-(trifluoromethyl)imidazo[1,2-b]pyridazin-2-yl)(4-(2-(trifluoromethyl)phenyl)piperidin-1-yl)methanone; (6-fluoroimidazo[1,2-b]pyridazin-2-yl)(4-(2-(trifluoromethyl)phenyl)piperidin-1-yl)methanone; (6-(pyrrolidin-1-yl)imidazo[1,2-b]pyridazin-2-yl)(4-(2-(trifluoromethyl)phenyl)piperidin-1-yl)methanone; (6-cyclopropylimidazo[1,2-b]pyridazin-2-yl)(4-(2-(trifluoromethyl)phenyl)piperidin-1-yl)methanone; (6-methoxyimidazo[1,2-b]pyridazin-2-yl)(4-(2-(trifluoromethyl)phenyl)piperidin-1-yl)methanone; (6-methylimidazo[1,2-b]pyridazin-2-yl)(4-(2-(trifluoromethyl)phenyl)piperidin-1-yl)methanone; (6-chloroimidazo[1,2-b]pyridazin-2-yl)(4-(2-(trifluoromethyl)phenyl)piperidin-1-yl)methanone; imidazo[1,2-b]pyridazin-2-yl(4-(2-(trifluoromethyl)phenyl)piperidin-1-yl)methanone; (6-chloro-2-methylimidazo[1,2-b]pyridazin-3-yl)(4-(2-(trifluoromethyl)phenyl)piperidin-1-yl)methanone; (1H-benzo[d]imidazol-2-yl)(4-(2-(trifluoromethyl)phenyl)piperidin-1-yl)methanone; (1H-imidazo[4,5-b]pyridin-2-yl)(4-(2-(trifluoromethyl)phenyl)piperidin-1-yl)methanone; (4-(5-fluoro-2-(trifluoromethyl)phenyl)piperidin-1-yl)(4,5,6,7-tetrahydro-1H-pyrazolo[3,4-c]pyridin-3-yl)methanone; (4-(3-fluoro-2-(trifluoromethyl)phenyl)piperidin-1-yl)(6-(2-methoxyethyl)-4,5,6,7-tetrahydro-1H-pyrazolo[3,4-c]pyridin-3-yl)methanone; 6-methyl-2-(4-(2-(trifluoromethyl)phenyl)piperidine-1-carbonyl)pyrimidine-4-carboxylic acid; methyl 6-methyl-2-(4-(2-(trifluoromethyl)phenyl)piperidine-1-carbonyl)pyrimidine-4-carboxylate; N-(cyclopropylsulfonyl)-2-(4-(2-(trifluoromethyl)phenyl)piperidine-1-carbonyl)benzamide; N-(phenylsulfonyl)-2-(4-(2-(trifluoromethyl)phenyl)piperidine-1-carbonyl)benzamide; N-(methylsulfonyl)-2-(4-(2-(trifluoromethyl)phenyl)piperidine-1-carbonyl)benzamide; 3-(4-(2-(trifluoromethyl)phenyl)piperidine-1-carbonyl)benzamide; 2-(4-(2-(trifluoromethyl)phenyl)piperidine-1-carbonyl)benzamide; 4-(4-(2-(trifluoromethyl)phenyl)piperidine-1-carbonyl)benzoic acid; 3-(4-(2-(trifluoromethyl)phenyl)piperidine-1-carbonyl)benzoic acid; 2-(4-(2-(trifluoromethyl)phenyl)piperidine-1-carbonyl)benzoic acid; 4-(4-(2-(tert-butyl)phenyl)piperidine-1-carbonyl)benzoic acid; 2-(4-(2-(tert-butyl)phenyl)piperidine-1-carbonyl)benzoic acid; 3-(4-(2-(tert-butyl)phenyl)piperidine-1-carbonyl)benzoic acid; 4-(4-(2-(trifluoromethyl)phenyl)piperidine-1-carbonyl)benzamide; 1-(3-(4-(2-(trifluoromethyl)phenyl)piperidine-1-carbonyl)-4,7-dihydroisothiazolo[5,4-c]pyridin-6(5H)-yl)ethan-1-one; (4,5,6,7-tetrahydroisothiazolo[5,4-c]pyridin-3-yl)(4-(2-(trifluoromethyl)phenyl)piperidin-1-yl)methanone; (4,5,6,7-tetrahydroisothiazolo[4,5-c]pyridin-3-yl)(4-(2-(trifluoromethyl)phenyl)piperidin-1-yl)methanone; 1-(3-(4-(2-(trifluoromethyl)phenyl)piperidine-1-carbonyl)-6,7-dihydroisoxazolo[4,5-c]pyridin-5(4H)-yl)ethan-1-one; 1-(3-(4-(2-(trifluoromethyl)phenyl)piperidine-1-carbonyl)-4,7-dihydroisoxazolo[5,4-c]pyridin-6(5H)-yl)ethan-1-one; (4,5,6,7-tetrahydroisoxazolo[4,5-c]pyridin-3-yl)(4-(2-(trifluoromethyl)phenyl)piperidin-1-yl)methanone; benzo[c]isothiazol-3-yl(4-(2-(trifluoromethyl)phenyl)piperidin-1-yl)methanone; benzo[d]thiazol-2-yl(4-(2-(trifluoromethyl)phenyl)piperidin-1-yl)methanone; benzo[d]isoxazol-3-yl(4-(2-(trifluoromethyl)phenyl)piperidin-1-yl)methanone; 1-(3-(4-(2-(trifluoromethyl)phenyl)piperidine-1-carbonyl)-6,7-dihydroisothiazolo[4,5-c]pyridin-5(4H)-yl)ethan-1-one; benzo[d]oxazol-2-yl(4-(2-(trifluoromethyl)phenyl)piperidin-1-yl)methanone; (3-methyloxetan-3-yl)(4-(2-(trifluoromethyl)phenyl)piperidin-1-yl)methanone; oxetan-3-yl(4-(2-(trifluoromethyl)phenyl)piperidin-1-yl)methanone; 2-(2-hydroxyphenyl)-1-(4-(2-(trifluoromethyl)phenyl)piperidin-1-yl)ethan-1-one; (4-(2-(tert-butyl)phenyl)piperidin-1-yl)(tetrahydrothiophen-2-yl)methanone; rac-tert-butyl (2R,3R)-2-(4-(2-(tert-butyl)phenyl)piperidine-1-carbonyl)-3-hydroxypyrrolidine-1-carboxylate; rac-tert-butyl (2R,4R)-2-(4-(2-(tert-butyl)phenyl)piperidine-1-carbonyl)-4-hydroxypyrrolidine-1-carboxylate 2-(2-oxo-2-(4-(2-(trifluoromethyl)phenyl)piperidin-1-yl)ethyl)phenyl sulfamate; (4-(2-(tert-butyl)phenyl)piperidin-1-yl)(1,1-dioxidotetrahydrothiophen-2-yl)methanone; rac-(4-(2-(tert-butyl)phenyl)piperidin-1-yl)((2R,3R)-3-hydroxypyrrolidin-2-yl)methanone; rac-(4-(2-(tert-butyl)phenyl)piperidin-1-yl)((2R,4R)-4-hydroxypyrrolidin-2-yl)methanone; rac-(R)-1-(2-(4-(2-(tert-butyl)phenyl)piperidine-1-carbonyl)pyrrolidin-1-yl)ethan-1-one; (6-bromo-1H-pyrrolo[3,2-b]pyridin-2-yl)(4-(2-(trifluoromethyl)phenyl)piperidin-1-yl)methanone; (5-morpholino-1H-pyrrolo[3,2-b]pyridin-2-yl)(4-(2-(trifluoromethyl)phenyl)piperidin-1-yl)methanone; (5-(1H-imidazol-1-yl)-1H-pyrrolo[3,2-b]pyridin-2-yl)(4-(2-(trifluoromethyl)phenyl)piperidin-1-yl)methanone; (5-chloro-1H-pyrrolo[3,2-b]pyridin-2-yl)(4-(2-(trifluoromethyl)phenyl)piperidin-1-yl)methanone; (5-fluoro-1H-pyrrolo[3,2-b]pyridin-2-yl)(4-(2-(trifluoromethyl)phenyl)piperidin-1-yl)methanone; (5-methyl-1H-pyrrolo[3,2-b]pyridin-2-yl)(4-(2-(trifluoromethyl)phenyl)piperidin-1-yl)methanone; (6-methoxy-1H-pyrrolo[3,2-b]pyridin-2-yl)(4-(2-(trifluoromethyl)phenyl)piperidin-1-yl)methanone; imidazo[1,2-a]pyridin-2-yl(4-(2-(trifluoromethyl)phenyl)piperidin-1-yl)methanone; (6-chloro-2-methylimidazo[1,2-b]pyridazin-3-yl)(4-(2-(trifluoromethyl)phenyl)piperidin-1-yl)methanone; imidazo[1,2-b]pyridazin-6-yl(4-(2-(trifluoromethyl)phenyl)piperidin-1-yl)methanone; (1H-pyrrolo[2,3-b]pyridin-2-yl)(4-(2-(trifluoromethyl)phenyl)piperidin-1-yl)methanone; (1H-pyrrolo[3,2-c]pyridin-2-yl)(4-(2-(trifluoromethyl)phenyl)piperidin-1-yl)methanone; (6-chloro-1H-pyrrolo[3,2-b]pyridin-2-yl)(4-(2-(trifluoromethyl)phenyl)piperidin-1-yl)methanone; (6-morpholino-1H-pyrrolo[3,2-b]pyridin-2-yl)(4-(2-(trifluoromethyl)phenyl)piperidin-1-yl)methanone; (6-(1H-imidazol-1-yl)-1H-pyrrolo[3,2-b]pyridin-2-yl)(4-(2-(trifluoromethyl)phenyl)piperidin-1-yl)methanone; (1-methyl-1H-pyrrolo[3,2-b]pyridin-2-yl)(4-(2-(trifluoromethyl)phenyl)piperidin-1-yl)methanone; (5-methoxy-1H-pyrrolo[3,2-b]pyridin-2-yl)(4-(2-(trifluoromethyl)phenyl)piperidin-1-yl)methanone; (6-fluoro-1H-pyrrolo[3,2-b]pyridin-2-yl)(4-(2-(trifluoromethyl)phenyl)piperidin-1-yl)methanone; (1H-imidazo[4,5-b]pyridin-2-yl)(4-(2-(trifluoromethyl)phenyl)piperidin-1-yl)methanone; (6-methyl-1H-pyrrolo[3,2-b]pyridin-2-yl)(4-(2-(trifluoromethyl)phenyl)piperidin-1-yl)methanone; (1H-indol-2-yl)(4-(2-(trifluoromethyl)phenyl)piperidin-1-yl)methanone; (1H-pyrrolo[3,2-b]pyridin-2-yl)(4-(2-(trifluoromethyl)phenyl)piperidin-1-yl)methanone; (1H-pyrrolo[2,3-c]pyridin-2-yl)(4-(2-(trifluoromethyl)phenyl)piperidin-1-yl)methanone; (1H-pyrazol-3-yl)(4-(2-(trifluoromethyl)phenyl)piperidin-1-yl)methanone; (1H-1,2,3-triazol-5-yl)(4-(2-(trifluoromethyl)phenyl)piperidin-1-yl)methanone; pyrazin-2-yl(4-(2-(trifluoromethyl)phenyl)piperidin-1-yl)methanone; (6-methoxypyridazin-3-yl)(4-(2-(trifluoromethyl)phenyl)piperidin-1-yl)methanone; (6-methylpyridazin-3-yl)(4-(2-(trifluoromethyl)phenyl)piperidin-1-yl)methanone; (4-methyl-1,2,3-thiadiazol-5-yl)(4-(2-(trifluoromethyl)phenyl)piperidin-1-yl)methanone; (6-chloropyridazin-3-yl)(4-(2-(trifluoromethyl)phenyl)piperidin-1-yl)methanone; pyridazin-3-yl(4-(2-(trifluoromethyl)phenyl)piperidin-1-yl)methanone; pyridazin-4-yl(4-(2-(trifluoromethyl)phenyl)piperidin-1-yl)methanone; 4-(2-(trifluoromethyl)phenyl)piperidine-1-carboxylic acid; or 3-oxo-3-(4-(2-(trifluoromethyl)phenyl)piperidin-1-yl)propanoic acid.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (II):

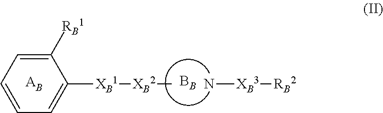

wherein
ring A_{B} is benzene optionally further substituted;
R_{B}¹ is an optionally substituted branched C₃₋₆ alkyl group;
X_{B}¹ is O, S, SO, SO₂ or NH;
X_{B}² is a bond or a C₁-C₃ alkylene group;
ring B_{B} is azetidine, pyrrolidine or piperidine;
X_{B}³ is CO or SO₂;
R_{B}² is a substituent;
or a pharmaceutically acceptable salt thereof.

In some embodiments, the compound of Formula (II) has the structure

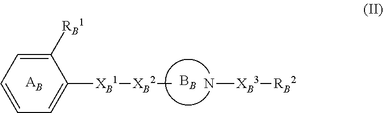

wherein
ring A_{B} is benzene optionally further substituted;
R_{B}¹ is an optionally substituted branched C₃-C₆ alkyl group
X_{B}¹ is O, S, SO, SO₂ or NH;
X_{B}² is a bond or a C₁-C₃ alkylene group;
ring B_{B} is azetidine or piperidine;
X_{B}³ is CO or SO₂;
R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom,
or a pharmaceutically acceptable salt thereof.

In some embodiments, the compound of Formula (II) is 4-(3-(2-tert-Butylphenoxy)azetidin-1-yl)-4-oxobutanoic acid, 3-{3-[(2-tert-Butyl-4-fluorophenoxy)methyl]azetidin-1-yl}-3-oxopropanoic acid, 2-{[3-(2-tert-Butyl-4-chlorophenoxy)azetidin-1-yl]carbonyl}pyridine, 4-[3-(2-tert-Butyl-4-chlorophenoxy)azetidin-1-yl]-4-oxobutanoic acid, {3-[(2-tert-Butyl-4-chlorophenoxy)methyl]azetidin-1-yl}(oxo)acetic acid, {3-[(2-tert-butylphenoxy)methyl]azetidin-1-yl}(oxo)acetic acid, 3-{3-[(2-tert-butylphenoxy)methyl]azetidin-1-yl}-3-oxopropanoic acid, {4-[(2-tert-Butyl-4-chlorophenoxy)methyl]piperidin-1-yl}(oxo)acetic acid, [4-(2-tert-butylphenoxy)piperidin-1-yl](oxo)acetic acid, or {4-[(2-tert-butylphenoxy)methyl]piperidin-1-yl}(oxo)acetic acid, or a pharmaceutically acceptable salt thereof.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (III)

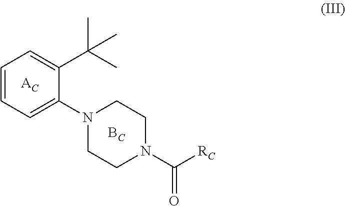

wherein
ring A_{C} is a benzene ring optionally further substituted;
ring B_{C} is a piperazine ring optionally further substituted; and
R_{C} is a substituent,
or a pharmaceutically acceptable salt thereof.

In some embodiments, the compound of Formula (III) has the structure

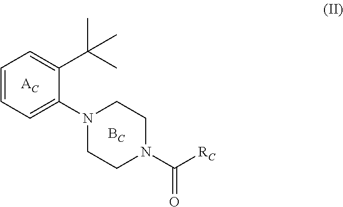

wherein
ring A_{C} is a benzene ring optionally substituted by 1 to 3 substituents selected from the group consisting of (a) a halogen atom, and (b) a C₁₋₆ alkyl group;
ring B_{C} is a piperazine ring optionally substituted by 1 to 3 substituents selected from the group consisting of (a) a halogen atom, (b) a C₁-C₆ alkyl group optionally substituted by 1 to 3 halogen atoms, and (c) a C₁-C₆ alkoxy group optionally substituted by 1 to 3 halogen atoms; and
R_{C} is (1) an optionally substituted C₁-C₁₀ alkyl group, (2) an optionally substituted C₆-C₁₄ aryl group, (3) an optionally substituted 5-or 6-membered aromatic heterocyclic group, (4) an optionally substituted amino group, (5) an optionally substituted carboxy group, or (6) an optionally substituted carbamoyl group,
or a pharmaceutically acceptable salt thereof.

In some embodiments, the compound of Formula (III) is N-{[4-(2-tert-Butylphenyl)piperazin-1-yl]carbonyl}glycine, 3-[4-(2-tert-Butylphenyl)piperazin-1-yl]-3-oxopropanoicacid, [4-(2-tert-Butyl-4-chlorophenyl)piperazin-1-yl](oxo)acetic acid, 5-{2-[4-(2-tert-Butylphenyl)piperazin-1-yl]-2-oxoethyl}imidazolidine-2,4-dione, [(5-{[4-(2-tert-Butylphenyl)piperazin-1-yl]carbonyl}isoxazol-3-yl)oxy]acetic acid, or a pharmaceutically acceptable salt thereof.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (IV)

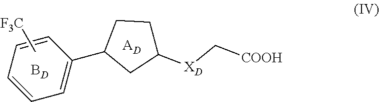

wherein
ring A_{D} is a 5-membered non-aromatic heterocycle optionally further substituted by one substituent;
ring B_{D} is an optionally further substituted benzene ring; and
X_{D} is a bond, O, CH₂O, OCH₂, CH₂, (CH₂)₂, S, CH₂S, SCH₂, S(O), CH₂S(O), S(O)CH₂, S(O)₂, CH₂S(O)₂ or S(O)₂CH₂
or a pharmaceutically acceptable salt thereof.

In some embodiments, the compound of Formula (IV) has the structure

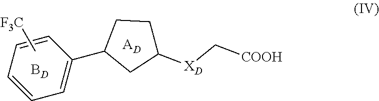

wherein
ring A_{D} is a 5-membered non-aromatic heterocycle optionally further substituted by one oxo group;
ring B_{D} is a benzene ring optionally further substituted by 1 to 4 substituents; and
X_{D} is O, CH₂O, OCH₂, CH₂, (CH₂)₂, S, CH₂S, SCH₂, S(O), CH₂S(O), S(O)CH₂, S(O)₂, CH₂S(O)₂ or S(O)₂CH₂,
or a pharmaceutically acceptable salt thereof.

In some embodiments, the compound of Formula (IV) is ({(3S)-1-[3,5-Bis(trifluoromethyl)phenyl]pyrrolidin-3-yl}oxy)acetic, ({1-[4-Chloro-3-(trifluoromethyl)phenyl]pyrrolidin-3-yl}sulfanyl)acetic acid, 3-{(2R,5S)-5-[3,5-Bis(trifluoromethyl)phenyl]tetrahydrofuran-2-yl}propanoic acid, or a pharmaceutically acceptable salt thereof.

Some embodiments provided herein describe an RBP4 inhibitor, or a pharmaceutically acceptable salt, solvate, polymorph, prodrug, metabolite, N-oxide, stereoisomer, or isomer thereof, for use in treating age-related macular degeneration, having the structure of Formula (V)

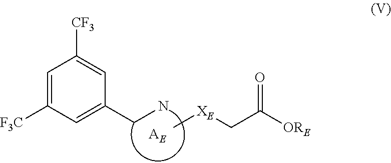

wherein
ring A_{E} is an optionally further substituted monocyclic nitrogen-containing aromatic heterocycle;
X_{E} is CH₂ or O; and
R_{E} is a hydrogen atom or a C₁₋₆ alkyl group,
or a pharmaceutically acceptable salt thereof.

In some embodiments, the compound of Formula (V) has the structure

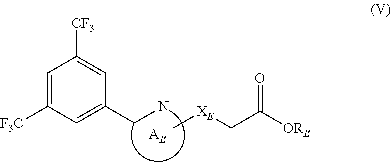

wherein
ring A_{E} is a pyrazole ring, a pyridine ring, an oxazole ring, an imidazole ring, or a pyrimidine ring;
X_{E} is S, optionally substituted alkylene, or O; and
R_{E} is a hydrogen atom or a C₁-C₆ alkyl group;
or a pharmaceutically acceptable salt thereof.

In some embodiments, the compound of Formula (V) is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, or 3-(3-(3,5-bis(trifluoromethyl)phenyl)-1H-pyrazol-1-yl)butanoic acid.

In some embodiments, the RBP4 inhibitor is an RBP4 inhibitor provided in Table B.

TABLE B
RBP4 Inhibitors

| Compound | | |
|---|---|---|
| No. | Name | Structure |
| 1 | 1-(3-(4-(3,4-difluoro-2- (trifluoromethyl)phenyl)piperidine-1- carbonyl)-1,4,5,7-tetrahydro-6H- pyrazolo[3,4-c]pyridin-6-yl)ethan-1-one | 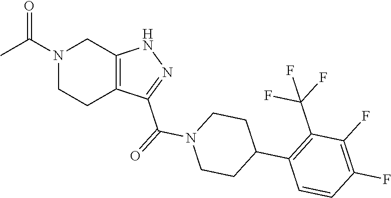 [SMILES: CC(=O)N1CCc2c(C(=O)N3CCC(c4ccc(F)c(F)c4C(F)(F)F)CC3)n[nH]c2C1] |
| 2 | 2-(3-((2-(tert-butyl)-4- chlorophenoxy)methyl)azetidin-1-yl)-2- oxoacetic acid | 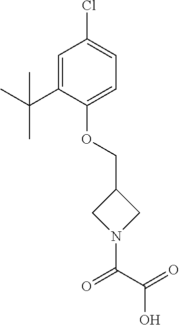 [SMILES: CC(C)(C)c1cc(Cl)ccc1OCC1CN(C(=O)C(=O)O)C1] |
| 3 | 3-(3-(2-(tert-butyl)-4-fluorophenoxy)azetidin- 1-yl)-3-oxopropanoic acid | 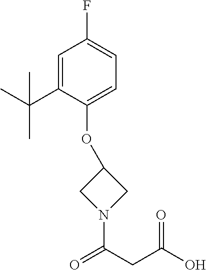 [SMILES: CC(C)(C)c1cc(F)ccc1OC1CN(C(=O)CC(=O)O)C1] |
| 4 | 4-(3-(2-(tert-butyl)-4-fluorophenoxy)azetidin- 1-yl)-4-oxobutanoic acid | 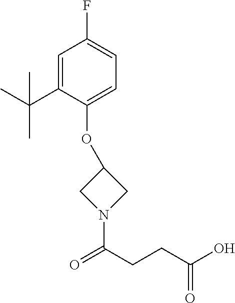 [SMILES: CC(C)(C)c1cc(F)ccc1OC1CN(C(=O)CCC(=O)O)C1] |
| 5 | (4-(2-(tert-butyl)phenoxy)piperidin-1- yl)(pyridin-2-yl)methanone | 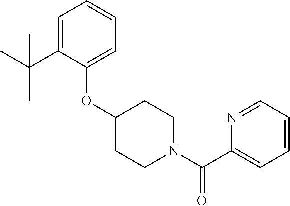 [SMILES: CC(C)(C)c1ccccc1OC1CCN(C(=O)c2ccccn2)CC1] |
| 6 | 1-(3-(2-(tert-butyl)phenoxy)azetidine-1- carbonyl)cyclopropane-1-carboxylic acid | 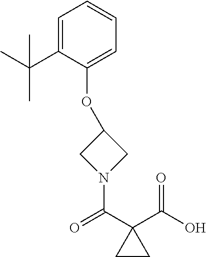 [SMILES: CC(C)(C)c1ccccc1OC1CN(C(=O)C2(C(=O)O)CC2)C1] |
| 7 | (3-(2-(tert-butyl)-4-fluorophenoxy)azetidin-1- yl)(pyridin-2-yl)methanone | 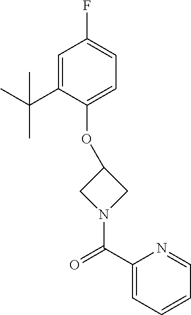 [SMILES: CC(C)(C)c1cc(F)ccc1OC1CN(C(=O)c2ccccn2)C1] |
| 8 | 3-(4-(2-(tert-butyl)phenyl)piperazin-1-yl)-3- oxopropanoic acid | 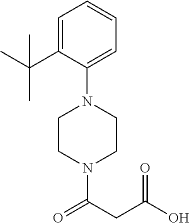 [SMILES: CC(C)(C)c1ccccc1N1CCN(C(=O)CC(=O)O)CC1] |
| 9 | 3-(4-(4-bromo-2-(tert-butyl)phenyl)piperazin- 1-yl)-3-oxopropanoic acid | 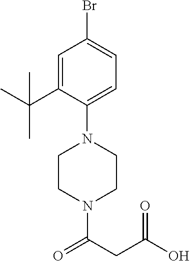 [SMILES: CC(C)(C)c1cc(Br)ccc1N1CCN(C(=O)CC(=O)O)CC1] |
| 10 | 2-(4-(2-(tert-butyl)phenyl)piperazin-1-yl)-2- oxoacetic acid | 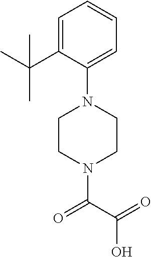 [SMILES: CC(C)(C)c1ccccc1N1CCN(C(=O)C(=O)O)CC1] |
| 11 | 4-(2-(tert-butyl)phenyl)-N-phenylpiperazine- 1-carboxamide | 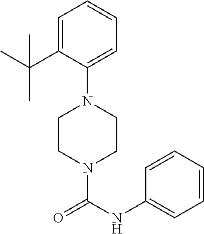 [SMILES: CC(C)(C)c1ccccc1N1CCN(C(=O)Nc2ccccc2)CC1] |
| 12 | (4-(2-(tert-butyl)phenyl)piperazin-1-yl)(1H- imidazol-4-yl)methanone | 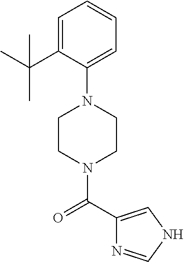 [SMILES: CC(C)(C)c1ccccc1N1CCN(C(=O)c2c[nH]cn2)CC1] |
| 13 | 2-((1-(3,5- bis(trifluoromethyl)phenyl)pyrrolidin-3- yl)thio)acetic acid | 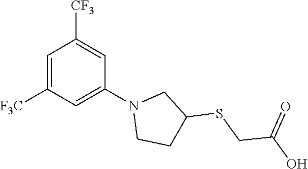 [SMILES: O=C(O)CSC1CCN(c2cc(C(F)(F)F)cc(C(F)(F)F)c2)C1] |
| 14 | 2-((1-(3,5- bis(trifluoromethyl)phenyl)pyrrolidin-3- yl)oxy)acetic acid | 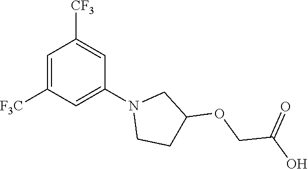 [SMILES: O=C(O)COC1CCN(c2cc(C(F)(F)F)cc(C(F)(F)F)c2)C1] |
| 15 | 3-(5-(3,5- bis(trifluoromethyl)phenyl)tetrahydrofuran-2- yl)propanoic acid | 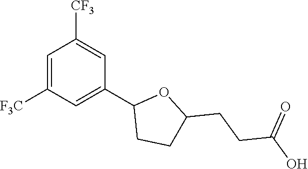 [SMILES: O=C(O)CCC1CCC(c2cc(C(F)(F)F)cc(C(F)(F)F)c2)O1] |
| 16 | 2-((4-(3,5-bis(trifluoromethyl)phenyl)oxazol- 2-yl)thio)acetic acid | 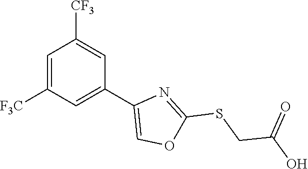 [SMILES: O=C(O)CSc1nc(-c2cc(C(F)(F)F)cc(C(F)(F)F)c2)co1] |
| 17 | 2-((6-(3,5-bis(trifluoromethyl)phenyl)pyridin- 3-yl)thio)acetic acid | 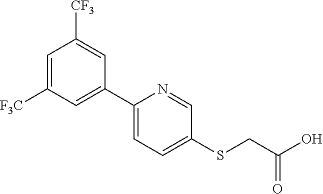 [SMILES: O=C(O)CSc1ccc(-c2cc(C(F)(F)F)cc(C(F)(F)F)c2)nc1] |
| 18 | ethyl 2-((6-(3,5- bis(trifluoromethyl)phenyl)pyridin-3- yl)thio)acetate | 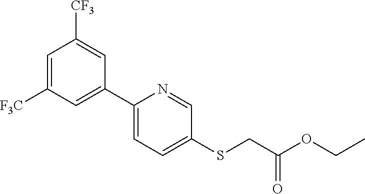 [SMILES: CCOC(=O)CSc1ccc(-c2cc(C(F)(F)F)cc(C(F)(F)F)c2)nc1] |
| 19 | 3-(3-(3,5-bis(trifluoromethyl)phenyl)-1H- pyrazol-1-yl)butanoic acid | 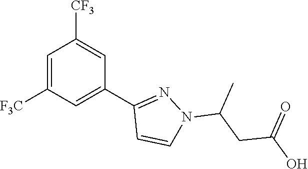 [SMILES: CC(CC(=O)O)n1ccc(-c2cc(C(F)(F)F)cc(C(F)(F)F)c2)n1] |
| 20 | 3-(4-(3-fluoro-2- (trifluoromethyl)phenyl)piperidine-1- carbonyl)-[1,2,4]triazolo[4,3-a]pyridine-6- carbonitrile | 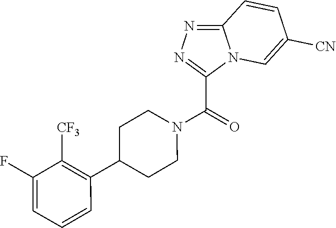 [SMILES: N#Cc1ccc2nnc(C(=O)N3CCC(c4cccc(F)c4C(F)(F)F)CC3)n2c1] |

### Preparation of Compounds

The compounds used in the chemical reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. MenloPark, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Alternatively, specific and analogous reactants can be identified through the indices of known chemicals and reactions prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the heterocyclic RBP4 inhibitory compound described herein is P. H. Stahl & C. G. Wermuth “Handbook of Pharmaceutical Salts”, Verlag Helvetica Chimica Acta, Zurich, 2002.

### Retinol Binding Protein 4 (RBP4)

Retinol-binding protein 4 (RBP4), the sole retinol transporter in blood, is secreted from adipocytes and the liver. Lowering levels of RBP4 can lead to reduction in the accumulation of lipofuscin that leads to vision loss in diseases like Age-Related Macular Degeneration or dry (atrophic) Age-Related Macular Degeneration. In some instances, lowering RBP4 reduces the accumulation of lipofuscin in the retina. In some embodiments, compounds and formulations described herein lower serum or plasmaRBP4 and thus delay or stop vision loss from excessive accumulation of lipofuscin in the retina. In some embodiments, compounds and formulations described herein lower serum or plasmaRBP4 and thus delay or stop vision loss from Age-Related Macular Degeneration.

In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 20% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 25% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 30% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 48 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reducedby at least 10 mg/dL. In some embodiments, 36 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reducedby at least 10 mg/dL. In some embodiments, 24 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reducedby at least 10 mg/dL. In some embodiments, 12 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 6 hours after administration of an RBP4 inhibitor, a pharmaceutically acceptable salt, solvate, polymorph, prodrug, metabolite, N-oxide, stereoisomer, or isomer thereof, the serum or plasma levels of RBP4 are reduced by at least 15 mg/dL.

### Methods of Treatment

In some embodiments, a compound disclosed herein is used to treat or ameliorate a disease associated with altered RBP4 pathways, such as Age-Related Macular Degeneration, when administered to a subject in need thereof. In some cases, a compound disclosed herein is used to treat or ameliorate the effects of a disease associated with altered RBP4 pathway, such as Age-Related Macular Degeneration, when administered to a subject in need thereof

### Age-Related Macular Degeneration

Age-related macular degeneration (AMD) is a common eye condition and a leading cause of vision loss among people age 50 and older. It causes damage to the macula, a small spot near the center of the retina and the part of the eye needed for sharp, central vision. As AMD progresses, a blurred area near the center of vision is a common symptom. Overtime, the blurred area may grow larger and the subject may develop blank spots in his or her central vision.

Some embodiments provided herein describe the use of the RBP4 inhibitors described herein for treating AMD in a subject in need thereof. In some embodiments, the RBP4 inhibitors inhibit AMD. In certain embodiments, the RBP4 inhibitors arrest development of AMD or its clinical symptoms. In certain embodiments, the RBP4 inhibitors reduce development of AMD or its clinical symptoms. In certain embodiments, the RBP4 inhibitors relieve the subject of AMD. In certain embodiments, the RBP4 inhibitors cause regression, reversal, or amelioration of AMD. In certain embodiments, the RBP4 inhibitors reduce the number, frequency, duration, or severity of AMD clinical symptoms.

In some embodiments, the RBP4 inhibitors are used prophylactically. In certain embodiments, the RBP4 inhibitors are used to prevent or reduce the risk of developing AMD. In certain embodiments, the RBP4 inhibitors cause the clinical symptoms of AMD to not develop in a subject who may be predisposed to AMD but who does not yet experience or display symptoms of AMD.

### Dry (Atrophic) Age-Related Macular Degeneration

Approximately 85% to 90% of the cases of macular degeneration are the “dry” (atrophic) type. It is estimated that 62.9 million individuals worldwide have this form of AMD; 8 million of them are Americans. Due to increasing life expectancy and current demographics this number is expected to triple by 2020. There is currently no FDA-approved treatment for dry AMD. Given the lack of treatment and high prevalence, development of drugs for dry AMD is of upmost importance. Clinically, atrophic AMD represents a slowly progressing neurodegenerative disorder in which specialized neurons (rod and cone photoreceptors) die in the central part of the retina called the macula. Histopathological and clinical imaging studies indicate that photoreceptor degeneration in dry AMD is triggered by abnormalities in the retinal pigment epithelium (RPE) that lies beneath photoreceptors and provides critical metabolic support to these light-sensing neuronal cells. Experimental and clinical data indicate that excessive accumulation of cytotoxic autofluorescent lipid-protein-retinoid aggregates (lipofuscin) in the RPE is a major trigger of dry AMD. The major cytotoxic component of RPE lipofuscin is pyridinium bisretinoid A2E. Additional cytotoxic bisretinoids are isoA2E, atRAL di-PE, and A2-DHP-PE. Formation of A2E and other lipofuscin bisretinoids, such as A2-DHP-PE (A2-dihydropyridine-phosphatidylethanolamine) and at RALdi-PE (all-trans-retinal dimer-phosphatidylethanolamine), begins in photoreceptor cells in a non-enzymatic manner and can be considered as a by-product of the properly functioning visual cycle.

Some embodiments provided herein describe the use of the RBP4 inhibitors described herein for treating dry (atrophic) AMD in a subject in need thereof. In some embodiments, the RBP4 inhibitors inhibit dry (atrophic) AMD. In certain embodiments, the RBP4 inhibitors arrest development of dry (atrophic) AMD or its clinical symptoms. In certain embodiments, the RBP4 inhibitors reduce development of dry (atrophic) AMD or its clinical symptoms. In certain embodiments, the RBP4 inhibitors relieve the subject of dry (atrophic) AMD. In certain embodiments, the RBP4 inhibitors cause regression, reversal, or amelioration of dry (atrophic) AMD. In certain embodiments, the RBP4 inhibitors reduce the number, frequency, duration, or severity of dry (atrophic) AMD clinical symptoms.

In some embodiments, the RBP4 inhibitors are used prophylactically. In certain embodiments, the RBP4 inhibitors are used to prevent or reduce the risk of developing dry (atrophic) AMD. In certain embodiments, the RBP4 inhibitors cause the clinical symptoms of dry (atrophic) AMD to not develop in a subject who may be predisposed to dry (atrophic) AMD but who does not yet experience or display symptoms of dry (atrophic) AMD.

### Pharmaceutical Compositions

In certain embodiments, the RBP4 inhibitory compound as described herein is administered as a pure chemical. In other embodiments, the RBP4 inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Provided herein is a pharmaceutical composition comprising at least one RBP4 inhibitory compound, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

One embodiment provides a pharmaceutical composition comprising an RBP4 inhibitor, or a pharmaceutically acceptable salt thereof, and a pharmaceutically acceptable excipient. In one embodiment, the pharmaceutical compositions are provided in a dosage form for oral administration, which comprise a compound provided herein, and one or more pharmaceutically acceptable excipients or carriers.

In certain embodiments, the RBP4 inhibitory compound is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the pharmaceutical compositions provided herein are formulated for oral administration in tablet, capsule, powder, or liquid form. In some embodiments, a tablet comprises a solid carrier or an adjuvant. Liquid pharmaceutical compositions generally comprise a liquid carrier such as water, petroleum, animal or vegetable oils, mineral oil, or synthetic oil. In some embodiments, physiological saline solution, dextrose or other saccharide solution, or glycols are optionally included. In some embodiments, a capsule comprises a solid carrier such as gelatin.

In another embodiment, the pharmaceutical compositions are provided in a dosage form for parenteral administration, which comprise a compound provided herein, and one or more pharmaceutically acceptable excipients or carriers. Where pharmaceutical compositions are formulated for intravenous, cutaneous or subcutaneous injection, the active ingredient is in the form of a parenterally acceptable aqueous solution, which is pyrogen-free and has a suitable pH, isotonicity, and stability. Those of relevant skill in the art are well able to prepare suitable solutions using, for example, isotonic vehicles, such as Sodium Chloride injection, Ringer's injection, or Lactated Ringer's injection. In some embodiments, preservatives, stabilizers, buffers, antioxidants, and/or other additives are included.

In yet another embodiment, the pharmaceutical compositions are provided in a dosage form for topical administration, which comprise a compound provided herein, and one or more pharmaceutically acceptable excipients or carriers.

### Methods of Dosing and Treatment Regimens

The dose of the composition comprising at least one RBP4 inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

In one embodiment, the compounds described herein, or a pharmaceutically acceptable salt thereof, are used in the preparation of medicaments for the treatment of diseases or conditions in a mammal that would benefit from administration of any one of the compounds disclosed. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, in which the mammal previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Oral doses typically range from about 1.0 mg to about 1000 mg, one to four times, or more, per day. In general, however, doses employed for adult human treatment are typically in the range of 0.01 mg to 5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED50 with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal. In some embodiments, an RBP4 inhibitor, a pharmaceutically acceptable salt, solvate, polymorph, prodrug, metabolite, N-oxide, stereoisomer, or isomer thereof is administered orally or parenterally to the subject in need thereof. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration. In some embodiments, an RBP4 inhibitor, a pharmaceutically acceptable salt, solvate, polymorph, prodrug, metabolite, N-oxide, stereoisomer, or isomer thereof is administered orally or intravenously to a subject in need thereof. In some embodiments, an RBP4 inhibitor, a pharmaceutically acceptable salt, solvate, polymorph, prodrug, metabolite, N-oxide, stereoisomer, or isomer thereof is administered orally to a subject in need thereof. In some embodiments, an RBP4 inhibitor, a pharmaceutically acceptable salt, solvate, polymorph, prodrug, metabolite, N-oxide, stereoisomer, or isomer thereof is administered intravenously to a subject in need thereof.

In any of the aforementioned aspects are further embodiments comprising single administrations of the effective amount of the compound, including further embodiments in which (i) the compound is administered once a day; or (ii) the compound is administered to the mammal multiple times over the span of one day, e.g., two, three, four or more times daily. In some embodiments, the RBP4 inhibitory compounds described herein are administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the RBP4 inhibitory compounds described herein, or a pharmaceutically acceptable salt, solvate, polymorph, prodrug, metabolite, N-oxide, stereoisomer, or isomer thereof, are administered daily.

In any of the aforementioned aspects are further embodiments comprising multiple administrations of the effective amount of the compound, including further embodiments in which (i) the compound is administered continuously or intermittently: as in a single dose; (ii) the time between multiple administrations is every 3 hours; (iii) the time between multiple administrations is every 6 hours; (iv) the compound is administered to the mammal every 8 hours; (v) the compound is administered to the mammal every 12 hours; or (vi) the compound is administered to the mammal every 24 hours.

In certain embodiments wherein a patient's status does improve, the dose of drug being administered is temporarily reduced or temporarily suspended for a certain length of time (e.g., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 7 days. In one embodiment, the length of the drug holiday is 7 days. In one embodiment, the length of the drug holiday is 14 days. In one embodiment, the length of the drug holiday is 28 days.

### EXAMPLES

### Example 1: Biomarker Analysis in Age-Related Macular Degeneration

Design. Healthy and AMD subjects were recruited for this study. For AMD group, the inclusion criteria were subjects over 50 years old who was diagnosed with AMD in the primary eye as defined by the Age-Related Eye Disease Study (AREDS) Research Group. For the healthy group, subjects aged over 50 years old, with no eye disease and a best corrected visual acuity (BCVA) of 20 Early Treatment Diabetic Retinopathy Study (ETDRS) letters were included. Subjects with active hepatitis, chronic kidney disease or pregnant females were excluded from the study. Demographic data, age, height, weight, and body mass index (BMI), systemic condition including obesity (waist/hip ratio >0.90 for males or 0.85 for females or BMI>30 kg/m2), hyperlipidemia (triglycerides level >150 mg/dl or high-density lipoprotein cholesterol <35 mg/dl for male or <39 mg/dl for female), metabolic syndrome, prediabetes, diabetes mellitus, hypertension (blood pressure >140/90 mmHg), cardiovascular diseases, and non-alcoholic fatty liver diseases and steatohepatitis, were collected from medical records. All participants signed informed consent. The study protocol was approved by the Institutional Review Board at the Chang GungMedical Foundation (No. 201701197B0) and the study was carried out in compliance with the Declaration of Helsinki.

AMD Severity Grading. Ages at AMD diagnosis were collected from medical records and AMD disease duration were calculated as the time between diagnosis and study enrollment. Color fundus photography (nonmyd 8s; Kowa Company, Ltd, Nagoya, Japan), fundus autofluorescence (Spectralis HRA2; Heidelberg Engineering, Heidelberg, Germany), and spectral-domain optical coherence tomography (Spectralis HRA2; Heidelberg Engineering) were used to grade AMD severity according to the definitions in AREDS. Images were classified by experienced ophthalmologists (K.-J.C, W.-C.W, Y.-S.H., H.-D.C.) and verified by a senior ophthalmologist (C.-C.L.) at the Chang Gung Memorial Hospital, Linkou. Briefly, AREDS category 1 (C1) is defined as a few (1-15), small (<63 μm), or no drusen and without pigment changes. AREDS category 2 (C2, early AMD) is characterized by several small, few intermediate-sized (63-124 μm) drusen, and/or pigmentary changes in one or both eyes. AREDS category 3 (C3, intermediate AMD) is characterized by extensive (20 soft or 65 hard without any soft) intermediate-size drusen, one large (>125 μm) drusen, and/or geographic atrophy not involving the macula in one orboth eyes. AREDS category 4 (C4, advanced unilateral AMD) consists of the advanced dry form with geography atrophy involving macula or the exudative form with choroidal neovascularization in one eye.

Plasma RBP4Measurement. Peripheral venous blood was drawn from study subjects. Samples were separated into buffy coat and plasma in equal aliquots for storage in −80° C. freezers until analysis. Plasma RBP4 concentration was measured using a commercial enzyme-linked immunosorbent assay kit (R&D System, USA). Assay was validated with commercial human RBP4 recombinant protein (R&D System) and healthy human serum samples at multiple concentration points including the assay's maximum and minimum detection levels. The detection range of this assay was 0.224-100 ng/ml, and the intra-assay and inter-assay coefficients of variation for RBP4 were 5.25% and 4.54%, respectively.

ABCA4 Variant Analysis. DNA materials were isolated from buffy coat samples using QIAamp DNA mini kit (QIAGEN, Netherlands). Next generation sequencing of the DNA samples was performed on a MiniSeq with an AmpliSeq custom panel designed by Illumina (Illumina, USA). Sequence data were analyzed, and variant calling was performed using BaseSpace (Illumina). Sequence alignment was performed using BWA (0.7.13) on the reference genome GRCh37/hg19 and analyzed using samtools (1.3) and Picard (2.1.1). Variant calling was performed using gatk (1.6). Population databases, db SNP and ClinVar, were consulted for curated information based on existing studies and allele frequencies were obtained from gnomAD and TOPMed databases. In silico algorithms, including PolyPhen-2, PANTHER, and Condel, were used to analyze and predict functional effects of variants that have not been reported. Quantitative trait association modelling was performed using PLINK (1.90).

Statistical analyses. Differences in clinical variables between the four groups were analyzed by x² test for categorical variables or Kruskal-Wallis test for continuous variables. Direct comparisons between groups were carried out using Student's t-test. Simple linear and logistic regression were used for the continuous and categorical variables, respectively. Associations among severity grades, RBP4, ages and other variables of interest were analyzed using the Spearman rank correlation. Odds ratios (OR) were calculated using median-unbiased estimation. All statistical analyses were performed with R (3.6.0) and RStudio (11.442; RStudio Inc, USA) and confirmed with SAS software (Version, SAS Institute Inc., USA). The significance level, 0.05, was considered. The analyses details of ABCA4 are described in the previous section.

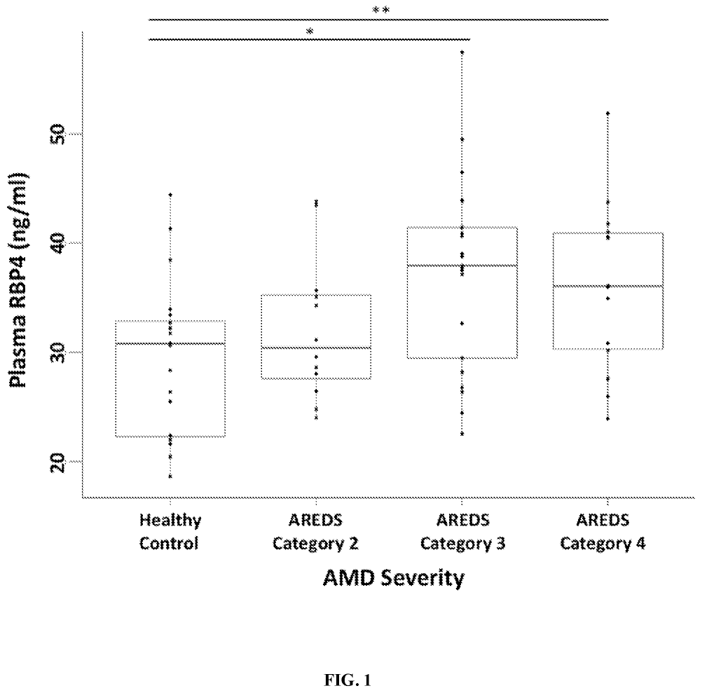

Cohort Characteristics. A total of 67 subjects including 20 healthy and 47 AMD (AREDS C2, C3, and C4) subjects were recruited at the Ophthalmology Department, Chang Gung Memorial Hospital, Linkou Medical Center, Taiwan from March 2018 to May 2019. Table 1 shows the demographics and clinical features of the study cohort. Subjects in the healthy group were significantly younger than the 3 AMTD groups due to difficulties in enrolling age-matched subjects. Metabolic syndrome was more common in the AMD groups compared to the healthy group. Subjects with AREDS C3 and AREDS C4 had significantly higher RBP4 levels (37.3±8.8 and 36.1±7.8 μg/ml, respectively) than the healthy controls (29.4±7.1 μg/ml; P=0.003 and P=0.002, respectively) (FIG. 1). The RBP4 level in AREDS C2 subjects (32.1±6.6 μg/ml) was non-significantly lower than that of AREDS C3 and C4 subjects, and AREDS C2 subjects showed smaller and less extensive drusens compared to subjects with worse AMD severity.

TABLE 1
Demographics and Clinical characteristics

| | | AREDS | AREDS | AREDS | |
|---|---|---|---|---|---|
| | Healthy | Category 2^{a} | Category 3^{a} | Category 4^{a} | P Value |
| n = | 20 | 12 | 21 | 14 | |
| Female, No. (%) | 15 (75) | 7 (58) | 11 (52) | 11 (79) | 0.31^{b} |
| Age, mean ± SD, y | 54.7 ± 4.1^{c, d, e} | 65.6 ± 6.8^{c} | 70.6 ± 7.3^{d} | 75.4 ± 7.8^{e} | <0.0001^{f} |
| BMI, mean ± SD, kg/m² | 22.4 ± 3.4 | 24.8 ± 3.4 | 23.7 ± 2.7 | 23.9 ± 3.2 | 0.13^{f} |
| Obesity, No. (%) | 1 (5) | 0 (0) | 0 (0) | 0 (0) | 0.67^{b} |
| Metabolic syndrome, No. (%) | 3 (15.0) | 6 (50) | 9 (42.9) | 9 (64.3) | 0.02^{b} |
| Cardiovascular diseases, | 0 (0) | 0 (0) | 0 (0) | 0 (0) | N/A |
| No. (%) | | | | | |
| Age at AMD diagnosis^{g}, | NA | 64.6 ± 7.7 | 68.4 ± 6.5 | 71.5 ± 8.7 | 0.16^{f} |
| mean ± SD, y | | | | | |
| AMD duration^{g}, | NA | 1.1 ± 2.0 | 2.2 ± 2.8 | 3.9 ± 5.3 | 0.31^{f} |
| mean ± SD, y | | | | | |
| Plasma RBP4 level, | 29.4 ± 7.1^{d, e} | 32.1 ± 6.6 | 37.3 ± 8.8^{d} | 36.1 ± 7.8^{e} | 0.02^{f} |
| mean ± SD, μg/ml | | | | | |

AMD, Age-related macular degeneration; AREDS, Age-Related Eye Disease Study; BMI, Body mass index; RBP4, Retinol binding protein 4.
^{a}As defined in the AREDS.
^{b}χ² test.
^{c}Significant difference between healthy and AREDS Category 2.
^{d}Significant difference between healthy and AREDS Category 3.
^{e}Significant difference between healthy and AREDS Category 4.
^{f}Kruskal-Wallis test, and post hoc test was performed by the Dunn's test if significant difference found between groups.
^{g}Healthy subjects were excluded from this analysis. AREDS Category 2 subjects were used as the reference for comparison.

The relationship between RBP4 and Severe AMD. Correlations of variables including age, plasma RBP4 level, BMI, and age at AMD diagnosis to AMD severity were analyzed using Spearman ranked correlation (Table 2). AMD severity was most significantly associated with age (p=0.68; P<0.0001), followed by plasma RBP4 level (p=0.36; P=0.001) and age at AMD diagnosis (p=−0.43; P<0.0001). Age was also significantly correlated with RPB4 level (p=0.44; P<0.001).

TABLE 2
Linear correlation coefficient between clinical variables.

| | | AMD | | Age at AMD | |
|---|---|---|---|---|---|
| | RBP4 | Severity^{b} | Age | Diagnosis | BMI |
| RBP4 | | 0.36** | 0.44*** | −0.10 | 0.24* |
| AMD Severityb | 0.36** | | 0.68*** | −0.43*** | 0.15 |
| Age | 0.44*** | 0.68*** | | −0.28* | 0.18 |
| Age at AMD | −0.10 | −0.43*** | −0.28* | | −0.27* |
| diagnosis | | | | | |
| BMI | 0.24* | 0.15 | 0.18 | −0.27* | |

AMD, Age-related macular degeneration; AREDS, Age-Related Eye Disease Study; BMI, Body mass index; RBP4, Retinol binding protein 4.
*P < .05.
**P < .01.
***P < .001.
^{a}Correlation coefficients calculated using Spearman rank correlation.
^{b}As defined in the AREDS.

Univariate logistic regressions were performed on variables significantly associated with AMD severity, namely age, plasmaRBP4 level, and age at AMD diagnosis. Only age (P<0.001) and RBP4 level (P=0.017) were still significantly associated with AMD severity while age at AMD diagnosis was no longer significant (P=0.057).

We explored plasmaRBP4 levels between the study cohort median (32.69 μg/ml) and disease group median (35.96 μg/ml) to identify a threshold of plasma RBP4 level that best distinguishes the severity of AMD. Three logistics regression models using plasma RBP4 levels of 33 μg/ml, 34 μg/ml, and 35 μg/ml as cut-off points were constructed, respectively. Using a plasma RBP4 level of 35 μg/ml (P=0.001) can better predict subjects with AREDS C4 than using 33 μg/ml (P=0.01) or 34 μg/ml (P=0.04) when healthy controls were used as the reference. Subjects with an RBP4 level higher than 35 μg/ml had significantly higher odds to present with AREDS C3 and AREDS C4 (OR 10.3 and 6.9, respectively) (Table 3). The odds ratio for each AREDS category when plasma RBP4 level is higher than 35 μg/ml.

TABLE 3

| | OR | 95% CI | P Value^{b} |
|---|---|---|---|
| Healthy | Reference | — | — |
| AREDS Category 2 | 2.7 | 0.5-17.9 | 0.44 |
| AREDS Category 3 | 10.3 | 2.4-58.8 | 0.002 |
| AREDS Category 4 | 6.9 | 1.4-42.8 | 0.027 |

AMD, Age-related macular degeneration; AREDS, Age-Related Eye Disease Study; CI, confidence interval; RBP4, Retinol binding protein 4.
^{a}As defined in the AREDS.
^{b}P values were calculated using logistic regression.

Furthermore, healthy control and AREDS C2 subjects were combined into no/mild AMD group and AREDS C3 and C4 subjects into severe AMD group, and analyzed the OR for severe AMD using a RBP4 level of 35 μg/ml as a cut-off point. The results demonstrated that subjects with a RBP4 level higher than 35 μg/ml were more likely to have severe AMD when using no/mild AMD as a reference group, with an OR of 6.06 (95% CI, 2.0-18.4; P=0.001).

Table 4 shows the results of the univariate and multivariate logistic regression model. The unadjusted odds for severe AMD by age was 1.26 (95% CI, 1.13-1.40), and increased by 11.6% as plasma RBP4 level increased by 1 μg/ml. Nevertheless, the odds of having severe AMD were only significant in age (OR=1.24; 95% CI, 1.12-1.39), while plasmaRBP4 level was not (OR=1.04; 95% CI, 0.95-1.14).

TABLE 4
Unadjusted and adjusted odds ratios for severe AMD^{a}

| | Unadjusted OR | | Adjusted OR | |
|---|---|---|---|---|
| | (95% CI) | P Value | (95% CI) | P Value |
| Age, y | 1.26 (1.13-1.40) | <0.001 | 1.24 (1.12-1.39) | <0.001 |
| Plasma RBP4 level, μg/ml | 1.12 (1.04-1.20) | 0.003 | 1.04 (0.95-1.14) | 0.40 |

AMD, Age-related macular degeneration; CI, confidence interval; OR, odds ratio; RBP4, Retinol binding protein 4.
^{a}Severe AMD group contains Age-Related Eye Disease Study (AREDS) category 3 and 4 subjects. Healthy and AREDS category 2 subjects were used as a reference group.

The Associations of ABCA4 Mutations with AMD Severity and RBP4 Levels. Only the AMD subjects were sequenced to detect genetic mutations in ABCA4 in an effort to identify association between mutations and AMD disease status. A total of 31 variants were identified in AMD subjects, including four missense mutations, one intronic splice acceptor variant, and one 3-primt UTR variant (Table 5). A majority of the variants (18/31) are consistent with previous publications. Many of these variants were found in only the severe form of AMID. Compared to allele frequencies reported in gnomAD and TOPMed databases, 24 variants appeared at a higher allele frequency in the AMID cohort. For example, the missense mutation, rs1800549, was found in eight of the 47 AMID patients, which is a higher frequency than the global allele frequency of 0.34%. Moreover, seven variants (namely, rs4147863, rs2275029, rs1800739, rs4147857, rs4147856, rs1801555, rs1801574) were found to be associated with a higher RBP4 level in 16 subjects when compared to subjects without these variants (37.8±7.7 vs 32.4±7.9 μg/ml; P=0.026).

TABLE 5
ABCA4 gene variants associated with AMD.

| | | | | | Allele Frequency (%) | | | |
|---|---|---|---|---|---|---|---|---|
| | | | | RBP4^{&} | AMD Subjects in | | Severe | |
| | | | | Mean ± | The Present | Reported in | AMD, No./ | |
| | | Protein | | SD | Study | the | total | |
| Variant | Mutation | Change | Type | (μg/ml) | (n = 47) | Database | (%) | |
| rs3747961 | c.*372= | NA | 3 Prime | 31.4 ± | 4.3 | 7.12 | 2/2 | (100) |
| | | | UTR | 4.7 | | | | |
| | | | variant | | | | | |
| rs6666652⁴⁴⁻⁴⁶ | c.6764G > T | p.S2255I | Missense | 34.8 ± | 8.5 | 15.96 | 4/4 | (100) |
| | | | | 7.0 | | | | |
| rs1800717⁴⁶ | NA | NA | Intronic | 34.8 ± | 8.5 | 13.61 | 4/4 | (100) |
| | | | | 7.0 | | | | |
| rs763108716 | c.6579C > T | p.F2193= | Synonymous | 34.9 | 2.1 | 0.01 | 1/1 | (100) |
| rs185601596 | NA | NA | Intronic | 47.8 | 2.1 | 0.01 | 1/1 | (100) |
| rs17110761⁴⁶ | NA | NA | Intronic | 33.3 ± | 19.2 | 12.42 | 8/9 | (89) |
| | | | | 9.7 | | | | |
| rs61748519⁴⁴ | c.6255C > T | p.L2085= | Synonymous | 37.5 ± | 4.3 | 0.35 | 1/2 | (50) |
| | | | | 14.5 | | | | |
| rs1801359^{44,49} | c.6249C > T | p.I2083= | Synonymous | 33.3 ± | 19.2 | 12.42 | 8/9 | (89) |
| | | | | 9.7 | | | | |
| rs145766145⁴⁷ | NA | NA | Intronic | 34.6 ± | 6.4 | 0.17 | 1/3 | (33) |
| | | | | 6.0 | | | | |
| rs76258939 | c.3626T > C | p.M1209T | Missense | 34.6 ± | 6.4 | 0.18 | 1/3 | (33) |
| | | | | 6.0 | | | | |
| rs200551567 | c.2841C > T | p.D947= | Synonymous | 28.4 | 2.1 | 0.01^{b} | 1/1 | (100) |
| rs754765164 | NA | NA | Intronic | 40.3 | 2.1 | 0.002^{b} | 1/1 | (100) |
| | | | splice | | | | | |
| | | | acceptor | | | | | |
| | | | variant | | | | | |
| rs201602424⁴⁴ | c.1614C > T | p.A538= | Synonymous | 38.8 ± | 2.1 | 0.04 | 1/1 | (100) |
| | | | | 12.7 | | | | |
| rs564661476 | NA | NA | Intronic | 30.9 ± | 4.3 | N/A^{c} | 2/2 | (100) |
| | | | insertion | 6.6 | | | | |
| rs4147831^{44,49} | c.1269C > T | p.H423= | Synonymous | 31.7 ± | 21.3 | 9.05 | 9/10 | (90) |
| | | | | 6.3 | | | | |
| rs6657239⁴⁴ | c.635G > A | p.R212H | Missense | 31.1 ± | 4.3 | 5.27 | 2/2 | (100) |
| | | | | 11.4 | | | | |
| rs2297632 | NA | NA | Intronic | 34.0 ± | 4.3 | 0.24 | 2/2 | (100) |
| | | | | 8.8 | | | | |
| rs1801555^{44,49}* | c.6285T > C | p.D2095= | Synonymous | 35.8 ± | 34.0 | 25.97 | 13/16 | (81) |
| | | | | 8.1 | | | | |
| rs1762114^{44,49} | c.6069T > C | p.I2023= | Synonymous | 34.2 ± | 91.5 | 80.22 | 35/43 | (81) |
| | | | | 8.1 | | | | |
| rs4147863*^{%} | NA | NA | Intronic | 37.8 ± | 21.3 | 16.68^{b} | 8/10 | (80) |
| | | | | 7.7 | | | | |
| rs2275029^{44,49}*^{%} | c.5844A > G | p.P1948= | Synonymous | 37.8 ± | 21.3 | 19.79 | 8/10 | (80) |
| | | | | 7.7 | | | | |
| rs1800739*^{%} | NA | NA | Intronic | 37.8 ± | 21.3 | 20.36 | 8/10 | (80) |
| | | | | 7.7 | | | | |
| rs4147857^{44,49}*^{%} | c.5814A > G | p.L1938= | Synonymous | 37.8 ± | 21.3 | 20.45 | 8/10 | (80) |
| | | | | 7.7 | | | | |
| rs4147856*^{%} | NA | NA | Intronic | 37.8 ± | 21.3 | 20.48 | 8/10 | (80) |
| | | | | 7.7 | | | | |
| rs1801574^{44,49}*^{%} | c.5682G > C | p.L1894= | Synonymous | 37.8 ± | 21.3 | 23.75 | 8/10 | (80) |
| | | | | 7.7 | | | | |
| rs55860151 | NA | NA | Intronic | 33.5 ± | 66.0 | 17.0^{b} | 22/31 | (71) |
| | | | | 7.7 | | | | |
| rs1800549¹⁹ | c.4283C > T | p.T1428M | Missense | 35.1 ± | 17.0 | 0.34 | 5/8 | (63) |
| | | | | 10.3 | | | | |
| rs3112831⁴⁴ | c.1268A > C | p.H423R | Synonymous | 35.8 ± | 51.1 | 26.04 | 20/24 | (83) |
| | | | | 8.4 | | | | |
| rs4147830 | NA | NA | Intronic | 32.9 ± | 85.1 | 47.22 | 29/40 | (73) |
| | | | | 7.8 | | | | |
| rs2297634⁴⁸ | NA | NA | Intronic | 33.6 ± | 89.4 | 49.54 | 30/42 | (71) |
| | | | | 8.0 | | | | |
| rs4847281⁴⁹ | c.141A > G | p.P47= | Synonymous | 33.5 ± | 76.6 | 98.91 | 28/36 | (78) |
| | | | | 7.6 | | | | |

AMD, Age-related macular degeneration; NA, not applicable; RBP4, Retinol binding protein 4.
a RBP4 level was calculated from all subjects with the variant.
^{b}Reported by TOPMed database.
^{c}No frequency reported by gnomAD or TOPMed databases.
d Significantly associated with elevated RBP4 level (P < 0.05).
e Linked variants.

Discussion Age-related macular degeneration (AND) is one of the most prevalent eye diseases in the senior population and is characterized by the presence of lipofuscin in the retinal pigment epithelium (RPE) layer. After photo excitation, the all-trans retinaldehyde attached to rhodopsin is released and forms N-retinylidene phosphatidylethanolamine (A2PE). A2PE is normally processed and reduced to all-trans retinol for reuse in another phototransduction reaction in a healthy person. However, A2PE gradually accumulates with age, allowing A2PE to form A2E, and the latter is a component of lipofuscin and induces cytotoxicity to photoreceptor and RPE cells. To avoid toxicity of A2E, a potential treatment approach is to reduce the formation and accumulation of A2E by modulating the visual cycle. One approach is to directly inhibit visual cycle proteins. Alternatively, A2E formation can be modulated by reducing retinol supply to the retina, which can be achieved by inhibiting RBP4, the primary transporter protein of retinol.

RBP4 is a 21 kDa protein with a single binding site for retinol. It is primarily produced in the liver to deliver retinol to peripheral tissues. Most research on RBP4 to date is in the fields of metabolic syndromes and cardiovascular diseases. A study in acute ischemic stroke found a significantly higher serum RBP4 level in stroke patients compared to normal controls (28.9 versus 23.7 μg/ml). Another 10-year study of 352 children showed significantly higher baseline RBP4 level in children with persistent metabolic syndrome (42.1 μg/ml) than those who never exhibited any sign of metabolic syndrome (32.7 μg/ml). Furthermore, baseline RBP4 predicted hyperglycemia, elevated triglyceride, elevated blood pressure, and insulin resistance in the study. Our study cohort also showed a similar trend consistent with these findings, with higher proportion of metabolic syndrome (43%-64%) and plasma RBP4 levels (32-37.3 μg/ml) found in the AMD groups compared to the healthy controls, which had only 15% with metabolic syndrome and a lower plasma RBP4 level at 29.4 μg/ml.

Apart from the associations of RBP4 and systemic conditions, the relationships between RBP4 and eye diseases was less investigated. Our study analyzed RBP4 and AMD and found a positive association between plasma RBP4 levels and AMD severity. We also established a cut-off concentration of plasma RBP4 at 35 μg/ml which significantly associated with higher odds of severe AMD. However, due to difficulties in recruiting age-matched healthy controls, there was a significant age gap between the AMD and control groups. As a result, the strong correlation between age and AMD severity overshadowed the correlation between RBP4 and AMD. Nonetheless, our results still see a positive trend between RPB4 and AMD which indicate a potential of utilizing plasma RBP4 levels to evaluate risk of severe AMD or using anti-RBP4 therapy in managing AMD.

Several RBP4 inhibitors have been proposed for the treatment AMD and Stargardt's Disease (STGD), a juvenile maculopathy that shares similar pathology as AMD. This theory was tested in a transgenic rodent model with ABCA4 mutations which reproduces the phenotype of lipofuscin accumulation and subsequent retinal dystrophy. Treating this mouse model with an RBP4 inhibitor can effectively reduce bisretinoid synthesis by 50% compared to untreated mice, and partially restored the expression of complement component 3, Complement Factor D, Complement Factor H, and C-reactive protein compared to the wild-type mice. As bisretinoid is a component of lipofuscin which included A2E, andthat the complement system and inflammation are both associated with AMD, this mouse model demonstrated the potential role of ABCA4 mutations in aggravating retinal degenerations.

Another RBP4 inhibitor, namely fenretinide, was used in a clinical trial to treat none-neovascular AMD patients. In this 2-year randomized controlled trial, 246 AMD patients with only geographic atrophy were treated with 100 mg or 300 mg of fenretinide or placebo. The results showed a reduction in the incidence of choroidal neovascularization onset in the treated patients, especially in the higher dose group. Moreover, in patients who achieved RBP4 levels below 1 μM (approximately 20 μg/ml) after fenretinide treatment, a lower serum RBP4 level was correlated with a slower rate of geographic atrophy progression. Although the overall cohort showed no statistically significant retardation of the growth rate of geographic atrophy due to insufficient number of subjects achieving a serum RBP4 level less than 1 μM, further clinical trials targeting RBP4 are ongoing.

Genetic profiling of the ABCA4 in this study revealed 6 variants associated with an RBP4 level greater than 35 μg/ml in AMD subjects, particularly in AREDS C3 and C4 groups. This finding suggests that there may be a genetic contribution to the elevated RBP4 level in patients with severe AMD. There were two subjects who possessed all 6 variants but were only diagnosed with less severe (AREDS C2) AMD. These two subjects were both newly diagnosed with AMD and possessed high RBP4 levels (34.3 and 43.5 μg/ml). These two patients may have a high likelihood of developing severer AMD in the future and suggested a close follow-up.

To our knowledge, this is the first study examining plasmaRBP4 level in AMD subjects and the correlation between RBP4 level and ABCA4 variants. The plasmaRBP4 threshold of 35 μg/ml is significantly associated with severe AMD and may have a genetic basis. The results from this pilot study implicated that RBP4 have a role in AMD and may potentially serve as a biomarker or a therapeutic target, especially when combined with ABCA4 genetic profiling.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method for treating age-related macular degeneration in an individual in need thereof comprising:
a) determining by an assay a level of retinol binding protein 4 (RBP4) in a sample from the individual, and
b) if the sample has the level of at least a threshold value of RBP4, then administering a therapy to reduce the level of RBP4 in the individual, wherein the threshold value is at least 25 μg/ml;
thereby treating the age-related macular degeneration in the individual.

2. The method of claim 1, wherein the threshold value of RBP4 is 35 μg/ml.

3. The method of claim 1, wherein the threshold value of RBP4 is 25-100 μg/ml.

4. The method of any one of claims 1-3, wherein the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof.

5. The method of claim 4, wherein the antibody assay comprises ELISA.

6. The method of any one of claims 1-5, further comprising:
c) determining by a second assay a level of vitamin A in a sample from the individual; and
d) if the sample has the level of at least a threshold value of vitamin A, then administering a therapy to reduce the level of vitamin A in the individual, wherein the threshold value of vitamin A is at least 150 ng/mL.

7. The method of claim 6, wherein the threshold value of vitamin A is about 225 ng/mL or about 390 ng/mL.

8. The method of claim 6, wherein the threshold value of vitamin A is from 150-500 ng/mL.

9. The method of any one of claims 6-8, wherein the second assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof.

10. The method of any one of claims 1-9, wherein the sample comprises a blood sample.

11. The method of claim 10, wherein the level is measured from plasma or serum derived from the blood sample.

12. The method of any one of claims 1-11, wherein if the level is below the threshold value of RBP4, then providing a recommendation to reassess the individual for macular degeneration after a period of time.

13. The method of any one of claims 1-12, wherein the therapy comprises a pharmaceutical composition comprising an RBP4 inhibitor or a compound which reduces blood RBP4 concentration in the individual.

14. The method of claim 13, wherein the pharmaceutical composition comprises a compound having the structure of Formula (I): 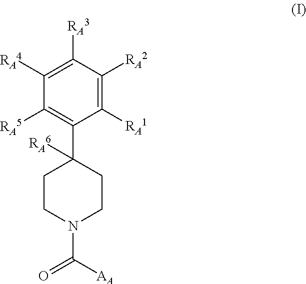
wherein:
R_{A}¹, R_{A}², R_{A}³, R_{A}⁴, and R_{A}⁵ are each independently H, halogen, CF₃ or C₁-C₄ alkyl, wherein two or more of R_{A}¹, R_{A}², R_{A}³, R_{A}⁴, and R_{A}⁵ are other than H;
R_{A}⁶ is H, OH, or halogen; and
A_{A} has the structure: 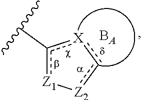
wherein
α, β, χ, and δ are each independently absent or present, and when present each is a bond;
X is C or N;
Z₁ is N;
Z₂ is N or NR_{A}⁹,
wherein R_{A}⁹ is H, C₁-C₄ alkyl, or oxetane;
B_{A} is a substituted or unsubstituted 5, 6, or 7 membered ring structure, or a pharmaceutically acceptable salt thereof.

15. The method of claim 14, wherein the compound has the structure

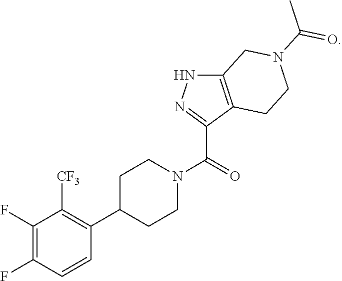 [SMILES: CC(=O)N1CCc2c(C(=O)N3CCC(c4ccc(F)c(F)c4C(F)(F)F)CC3)n[nH]c2C1]

16. The method of claim 14, wherein the compound has the structure

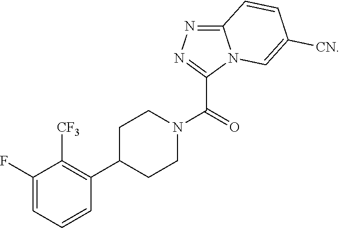 [SMILES: N#Cc1ccc2nnc(C(=O)N3CCC(c4cccc(F)c4C(F)(F)F)CC3)n2c1]

17. The method of claim 13, wherein the pharmaceutical composition comprises a compound having the structure of Formula (II): 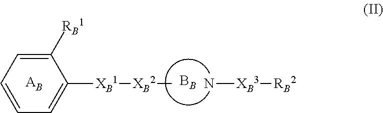
wherein:
ring A_{B} is benzene optionally further substituted;
R_{B}¹ is an optionally substituted branched C₃-C₆ alkyl group
X_{B}¹ is O, S, SO, SO₂ or NH;
X_{B}² is a bond or a C₁-C₃ alkylene group;
ring B_{B} is azetidine or piperidine;
X_{B}³ is CO or SO₂;
R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom,
or a pharmaceutically acceptable salt thereof.

18. The method of claim 17, wherein the compound is 4-(3-(2-tert-Butylphenoxy)azetidin-1-yl)-4-oxobutanoic acid, 3-{3-[(2-tert-Butyl-4-fluorophenoxy)methyl]azetidin-1-yl}-3-oxopropanoic acid, 2-{[3-(2-tert-Butyl 4-chlorophenoxy)azetidin-1-yl]carbonyl}pyridine, 4-[3-(2-tert-Butyl-4-chlorophenoxy)azetidin-1-yl]-4-oxobutanoic acid, {3-[(2-tert-Butyl-4-chlorophenoxy)methyl]azetidin-1-yl}(oxo)acetic acid, {3-[(2-tert-butylphenoxy)methyl]azetidin-1-yl}(oxo)acetic acid, 3-{3-[(2-tert-butylphenoxy)methyl]azetidin-1-yl}-3-oxopropanoic acid, {4-[(2-tert-Butyl-4-chlorophenoxy)methyl]piperidin-1-yl}(oxo)acetic acid, [4-(2-tert-butylphenoxy)piperidin-1-yl](oxo)acetic acid, or (4-[(2-tert-butylphenoxy)methyl]piperidin-1-yl 1(oxo)acetic acid, or a pharmaceutically acceptable salt thereof.

19. The method of claim 13, wherein the pharmaceutical composition comprises a compound having the structure of Formula (III): 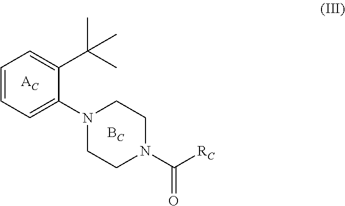
wherein
ring A_{C} is a benzene ring optionally substituted by 1 to 3 substituents selected from the group consisting of (a) a halogen atom, and (b) a C₁₋₆ alkyl group,
ring B_{C} is a piperazine ring optionally substituted by 1 to 3 substituents selected from the group consisting of (a) a halogen atom, (b) a C₁-C₆ alkyl group optionally substituted by 1 to 3 halogen atoms, and (c) a C₁-C₆ alkoxy group optionally substituted by 1 to 3 halogen atoms, and
R_{C} is (1) an optionally substituted C₁-C₁₀alkyl group, (2) an optionally substituted C₆-C₁₄ aryl group, (3) an optionally substituted 5- or 6-membered aromatic heterocyclic group, (4) an optionally substituted amino group, (5) an optionally substituted carboxy group, or (6) an optionally substituted carbamoyl group, or a pharmaceutically acceptable salt thereof.

20. The method of claim 19, wherein the compound is N-{[4-(2-tert-Butylphenyl)piperazin-1-yl]carbonyl}glycine, 3-[4-(2-tert-Butylphenyl)piperazin-1-yl]-3-oxopropanoic acid, [4-(2-tert-Butyl-4-chlorophenyl)piperazin-1-yl](oxo)acetic acid, 5-{2-[4-(2-tert-Butylphenyl)piperazin-1-yl]-2-oxoethyl}imidazolidine-2,4-dione, [(5-{[4-(2-tert-Butylphenyl)piperazin-1-yl]carbonyl}isoxazol-3-yl)oxy]acetic acid, or a pharmaceutically acceptable salt thereof.

21. The method of claim 13, wherein the pharmaceutical composition comprises a compound having the structure of Formula (IV): 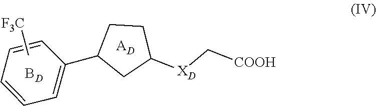
wherein
ring A_{D} is a 5-membered non-aromatic heterocycle optionally further substituted by one oxo group;
ring B_{D} is a benzene ring optionally further substituted by 1 to 4 substituents; and
X_{D} is O, CH₂O, OCH₂, CH₂, (CH₂)₂, S, CH₂S, SCH₂, S(O), CH₂S(O), S(O)CH₂, S(O)₂, CH₂S(O)₂ or S(O)₂CH₂, or a pharmaceutically acceptable salt thereof.

22. The method of claim 21, wherein the compound is ({(3S)-1-[3,5-Bis(trifluoromethyl)phenyl]pyrrolidin-3-yl}oxy)acetic, ({1-[4-Chloro-3-(trifluoromethyl)phenyl]pyrrolidin-3-yl}sulfanyl)acetic acid, 3-{(2R,5S)-5-[3,5-Bis(trifluoromethyl)phenyl]tetrahydrofuran-2-yl}propanoic acid, or a pharmaceutically acceptable salt thereof.

23. The method of claim 13, wherein the pharmaceutical composition comprises a compound having the structure of Formula (V): 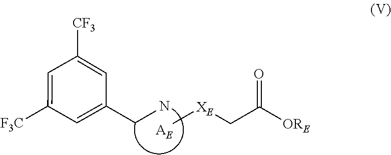
wherein
ring A_{E} is a pyrazole ring, a pyridine ring, an oxazole ring, an imidazole ring, or a pyrimidine ring,
X_{E} is S, optionally substituted alkylene, or O; and
R_{E} is a hydrogen atom or a C₁-C₆alkyl group;
or a pharmaceutically acceptable salt thereof.

24. The method of claim 23, wherein the compound is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, or 3-(3-(3,5-bis(trifluoromethyl)phenyl)-1H-pyrazol-1-yl)butanoic acid.

25. A method for treating age-related macular degeneration in an individual in need thereof comprising:
a) determining by an assay a level of vitamin A in a sample from the individual; and
b) if the sample has the level of at least a threshold value of vitamin A, then administering a therapy to reduce the level of vitamin A or a level of RBP4 in the individual, wherein the threshold value is at least 150 ng/ml;
thereby treating the age-related macular degeneration in the individual.

26. The method of claim 25, wherein the threshold value of vitamin A is about 225 ng/mL or about 390 ng/mL.

27. The method of claim 25, wherein the threshold value of vitamin A is 150-500 ng/mL.

28. The method of any one of claims 25-27, wherein the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof.

29. The method of claim 28, wherein the chromatographic assay comprises high performance liquid chromatography (HPLC), ultra-high performance liquid chromatography (UPLC), or liquid chromatography/mass spectrometry (LC-MS).

30. The method of any one of claims 25-29, wherein the sample comprises a blood sample.

31. The method of claim 30, wherein the level is measured from plasma or serum derived from the blood sample.

32. The method of any one of claims 25-31, wherein if the level is below the threshold value of vitamin A, then providing a recommendation to reassess the individual for macular degeneration after a period of time.

33. The method of any one of claims 25-32, wherein the therapy comprises a pharmaceutical composition comprising an RBP4 inhibitor or a compound which reduces blood RBP4 concentration in the individual.

34. A method for assessing the likelihood of, the severity of, or a diagnosis of age-related macular degeneration in an individual comprising one or more of:
a) determining by an assay if a level of retinol binding protein 4 (RBP4) in a sample from the individual is above a threshold level of RBP4;
b) determining the age and medical history of the individual; and
c) assessing the likelihood of developing, the severity of, or the diagnosis of age-related macular degeneration based on the level of RBP4 and optionally on the age and/or medical history of the individual,
wherein the threshold level of RBP4 is at least 25 μg/ml.

35. The method of claim 34, wherein the threshold value is 35 μg/ml.

36. The method of claim 34, wherein the threshold value is 25-100 μg/ml.

37. The method of any one of claims 34-36, wherein the method further comprises performing at least one of color fundus photography, fundus autofluorescence, spectral-domain optical coherence tomography, or microperimetry.

38. The method of any one of claims 34-37, further comprising classifying a progression of age-related macular degeneration.

39. The method of claim 38, wherein classifying comprises use of AREDS categories.

40. The method of any one of claims 34-39, wherein the assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof.

41. The method of claim 40, wherein the antibody assay comprises ELISA.

42. The method of any one of claims 34-41, wherein the sample comprises a blood sample.

43. The method of claim 42, wherein the level is measured from plasma or serum derived from the blood sample.

44. The method of any one of claims 34-43, wherein assessing the likelihood of developing macular degeneration comprises generating a risk score.

45. The method of claim 44, wherein if the risk score is above a threshold value, administering a pharmaceutical composition for treatment of macular degeneration.

46. The method of claim 45, further comprising selecting a dose of the pharmaceutical composition based on the level of RBP4.

47. The method of claim 44, wherein if the risk score is below a threshold value, providing a recommendation to reassess the individual for macular degeneration after a period of time.

48. A method for treating age-related macular degeneration in an individual in need thereof comprising:
a) determining by an assay the presence or absence of one or more genomic variants, wherein the one or more genomic variants comprises at least one of rs4147863, rs2275029, rs1800739, rs4147857, rs4147856, rs1801555, or rs1801574,
b) calculating a risk score for age-related macular degeneration using the presence or absence of the one or more genomic variants, and
c) administering a therapy to treat age-related macular degeneration in the individual.

49. The method of claim 48, wherein the one or more genomic variants comprises at least four of rs4147863, rs2275029, rs1800739, rs4147857, rs4147856, rs1801555, or rs1801574.

50. The method of claim 48 or 49, wherein the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281.

51. The method of any one of claims 48-50, further comprising determining the age or medical history of the individual.

52. The method of any one of claims 48-51, further comprising determining a level of retinol binding protein 4 (RBP4) in a sample from the individual.

53. The method of any one of claims 48-52, wherein the therapy comprises administering a pharmaceutical composition to the individual.

54. The method of claim 53, wherein the pharmaceutical composition comprises an RBP4 inhibitor or a compound which reduces blood RBP4 concentration in the individual.

55. The method of claim 53 or 54, wherein the pharmaceutical composition comprises a compound having the structure of Formula (I): 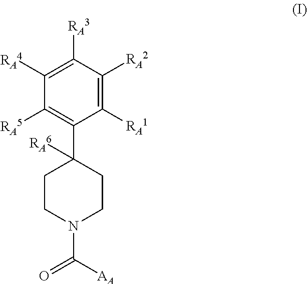
wherein:
R_{A}¹, R_{A}², R_{A}³, R_{A}⁴, and R_{A}⁵ are each independently H, halogen, CF₃ or C₁-C₄ alkyl, wherein two or more of R_{A}¹, R_{A}², R_{A}³, R_{A}⁴, and R_{A}⁵ are other than H;
R_{A}⁶ is H, OH, or halogen; and
A_{A} has the structure: 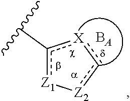
wherein
α, β, and δ are each independently absent or present, and when present each is a bond;
X is C or N;
Z₁ is N;
Z₂ is N or NR_{A}⁹,
wherein R_{A}⁹ is H, C₁-C₄ alkyl, or oxetane;
B_{A} is a substituted or unsubstituted 5, 6, or 7 membered ring structure,
or a pharmaceutically acceptable salt thereof.

56. The method of claim 55, wherein the pharmaceutical composition comprises a compound having the structure 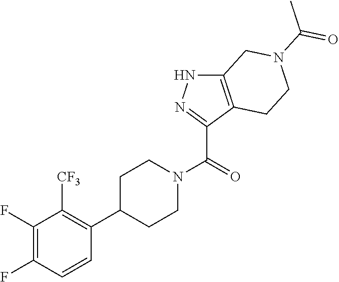 [SMILES: CC(=O)N1CCc2c(C(=O)N3CCC(c4ccc(F)c(F)c4C(F)(F)F)CC3)n[nH]c2C1]
or a pharmaceutically acceptable salt thereof.

57. The method of claim 55, wherein the pharmaceutical composition comprises a compound having the structure 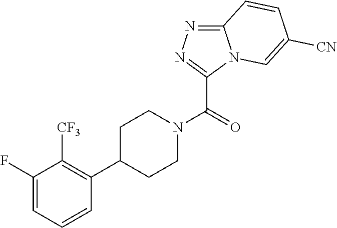 [SMILES: N#Cc1ccc2nnc(C(=O)N3CCC(c4cccc(F)c4C(F)(F)F)CC3)n2c1]
or a pharmaceutically acceptable salt thereof.

58. A method for assessing the likelihood of age-related macular degeneration in an individual in need thereof comprising:
a) extracting a protein fraction from a blood sample obtained from the individual;
b) extracting a nucleic acid fraction from the blood sample;
c) determining by a first assay a level of retinol binding protein 4 (RBP4) from the protein fraction;
d) determining by a second assay an allele frequency of one or more genomic variants from nucleic acids in the nucleic acid fraction, wherein the one or more genomic variants comprises at least one of rs4147863, rs2275029, rs1800739, rs4147857, rs4147856, rs1801555, or rs1801574; and
e) assessing the likelihood of age-related macular degeneration based on the level of RBP4 and the allele frequency of the one or more genomic variants.

59. The method of claim 58, wherein the first assay comprises an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof.

60. The method of claim 59, wherein the antibody assay comprises ELISA.

61. The method of any one of claims 58-60, wherein the level is measured from plasma or serum derived from the blood sample.

62. The method of any one of claims 58-61, wherein the one or more genomic variants comprises at least four of rs4147863, rs2275029, rs1800739, rs4147857, rs4147856, rs1801555, or rs1801574

63. The method of any one of claims 58-62, wherein the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3112831, rs4147830, rs2297634, or rs4847281

64. The method of any one of claims 58-63, further comprising determining the age or medical history of the individual.

65. The method of any one of claims 58-64, further comprising performing at least one of color fundus photography, fundus autofluorescence, spectral-domain optical coherence tomography, or microperimetry.

66. The method of any one of claims 58-65, further comprising classifying a progression of age-related macular degeneration.

67. The method of claim 66, wherein classifying comprises use of AREDS categories.

68. The method of any one of claims 58-67, wherein assessing the likelihood of age-related macular degeneration comprises generating a risk score.

69. A method for treating age-related macular degeneration in an individual in need thereof comprising:
a) providing a level of retinol binding protein 4 (RBP4), wherein the level was determined by an assay of a protein fraction of a sample from the individual;
b) providing an allele frequency of one or more genomic variants, wherein the allele frequency was determined by an assay performed on a nucleic acid fraction of the sample from the individual, and wherein the one or more genomic variants comprises at least one of rs4147863, rs2275029, rs1800739, rs4147857, rs4147856, rs1801555, or rs1801574; and
c) administering a therapy based on an assessment of the level of RBP4 and the allele frequency of the one or more genomic variants.

70. The method of claim 69, wherein the sample comprises a blood sample.

71. The method of claim 70, wherein the level is measured from plasma or serum derived from the blood sample.

72. The method of any one of claims 69-71, wherein the assay is an antibody assay, an electrophoresis assay, an immunoassay, a radioimmunoassay, a chromatographic assay, a mass spectrometry assay, a microarray based detection assay, a polymerase chain reaction assay, a sequencing assay, an immunohistochemistry assay, or any combination thereof.

73. The method of claim 72, wherein the antibody assay comprises ELISA.

74. The method of any one of claims 69-73, wherein the assessment comprises a diagnosis of age-related macular degeneration.

75. The method of any one of claims 69-74, wherein the assessment further comprises analysis of at least one of color fundus photography, fundus autofluorescence, spectral-domain optical coherence tomography, or microperimetry.

76. The method of any one of claims 69-75, wherein the assessment further comprises analysis of the age or medical history of the individual.

77. The method of any one of claims 69-76, wherein the one or more genomic variants comprises at least four of rs4147863, rs2275029, rs1800739, rs4147857, rs4147856, rs1801555, or rs1801574.

78. The method of any one of claims 69-77, wherein the one or more genomic variants comprises at least five of rs3747961, rs6666652, rs1800717, rs763108716, rs185601596, rs17110761, rs61748519, rs1801359, rs145766145, rs76258939, rs200551567, rs754765164, rs201602424, rs564661476, rs4147831, rs6657239, rs2297632, rs1801555, rs1762114, rs55860151, rs1800549, rs3l 12831, rs4147830, rs2297634, or rs4847281.

79. The method of any one of claims 69-78, wherein the therapy comprises administering a pharmaceutical composition to the individual.

80. The method of claim 79, wherein the pharmaceutical composition comprises an RBP4 inhibitor or a compound which reduces blood RBP4 concentration in the individual.

81. The method of claim 33, 45, 53, 54, 79, or 80 wherein the pharmaceutical composition comprises a compound having the structure of Formula (I): 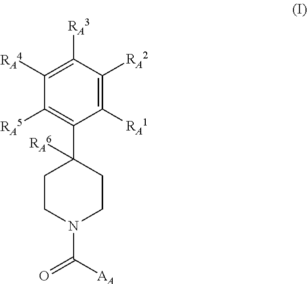
wherein:
R_{A}¹, R_{A}², R_{A}³, R_{A}⁴, and R_{A}⁵ are each independently H, halogen, CF₃ or C₁-C₄ alkyl, wherein two or more of R_{A}¹, R_{A}², R_{A}³, R_{A}⁴, and R_{A}⁵ are other than H;
R_{A}⁶ is H, OH, or halogen; and
A_{A} has the structure: 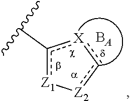
wherein
α, β, χ, and δ are each independently absent or present, and when present each is a bond;
X is C or N;
Z₁ is N;
Z₂ is N or NR_{A}⁹,
wherein R_{A}⁹ is H, C₁-C₄ alkyl, or oxetane;
B_{A} is a substituted or unsubstituted 5, 6, or 7 membered ring structure,
or a pharmaceutically acceptable salt thereof.

82. The method of claim 81, wherein the pharmaceutical composition comprises a compound having the structure 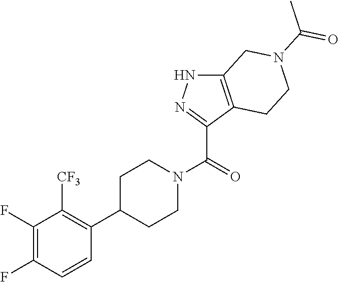 [SMILES: CC(=O)N1CCc2c(C(=O)N3CCC(c4ccc(F)c(F)c4C(F)(F)F)CC3)n[nH]c2C1]
or a pharmaceutically acceptable salt thereof.

83. The method of claim 81, wherein the pharmaceutical composition comprises a compound having the structure 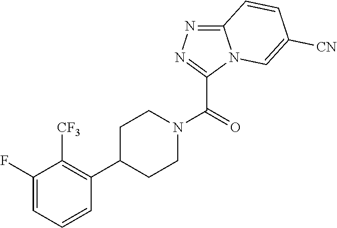 [SMILES: N#Cc1ccc2nnc(C(=O)N3CCC(c4cccc(F)c4C(F)(F)F)CC3)n2c1]
or a pharmaceutically acceptable salt thereof.

84. The method of claim 33, 45, 53, 54, 79, or 80, wherein the pharmaceutical composition comprises a compound having the structure of Formula (II): 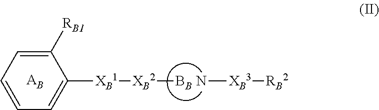
wherein:
ring A_{B} is benzene optionally further substituted;
R_{B}¹ is an optionally substituted branched C₃-C₆ alkyl group
X_{B}¹ is O, S, SO, SO₂ or NH;
X_{B}² is a bond or a C₁-C₃ alkylene group;
ring B_{B} is azetidine or piperidine;
X_{B}³ is CO or SO₂;
R_{B}² is an optionally substituted hydrocarbon group, an optionally substituted heterocyclic group, an optionally substituted hydroxy group, an optionally substituted mercapto group, a cyano group, a nitro group, an acyl group, or a halogen atom,
or a pharmaceutically acceptable salt thereof.

85. The method of claim 84, wherein the compound is 4-(3-(2-tert-Butylphenoxy)azetidin-1-yl)-4-oxobutanoic acid, 3-(3-[(2-tert-Butyl-4-fluorophenoxy)methyl]azetidin-1-yl)-3-oxopropanoicacid, 2-{[3-(2-tert-Butyl-4-chlorophenoxy)azetidin-1-yl]carbonyl}pyridine, 4-[3-(2-tert-Butyl-4-chlorophenoxy)azetidin-1-yl]-4-oxobutanoic acid, {3-[(2-tert-Butyl-4-chlorophenoxy)methyl]azetidin-1-yl)(oxo)acetic acid, (3-[(2-tert-butylphenoxy)methyl]azetidin-1-yl)(oxo)acetic acid, 3-(3-[(2-tert-butylphenoxy)methyl]azetidin-1-yl}-3-oxopropanoic acid, {4-[(2-tert-Butyl-4-chlorophenoxy)methyl]piperidin-1-yl}(oxo)acetic acid, [4-(2-tert-butylphenoxy)piperidin-1-yl](oxo)acetic acid, or {4-[(2-tert-butylphenoxy)methyl]piperidin-1-yl}(oxo)acetic acid, or a pharmaceutically acceptable salt thereof.

86. The method of claim 33, 45, 53, 54, 79, or 80, wherein the pharmaceutical composition comprises a compound having the structure of Formula (III): 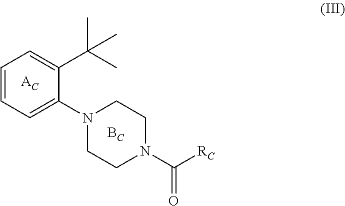
wherein
ring A_{C} is a benzene ring optionally substituted by 1 to 3 substituents selected from the group consisting of (a) a halogen atom, and (b) a C₁₋₄ alkyl group;
ring B_{C} is a piperazine ring optionally substituted by 1 to 3 substituents selected from the group consisting of (a) a halogen atom, (b) a C₁-C₆ alkyl group optionally substituted by 1 to 3 halogen atoms, and (c) a C₁-C₆ alkoxy group optionally substituted by 1 to 3 halogen atoms; and
R_{C} is (1) an optionally substituted C₁-C₁₀ alkyl group, (2) an optionally substituted C₆-C₁₄ aryl group, (3) an optionally substituted 5- or 6-membered aromatic heterocyclic group, (4) an optionally substituted amino group, (5) an optionally substituted carboxy group, or (6) an optionally substituted carbamoyl group, or a pharmaceutically acceptable salt thereof.

87. The method of claim 86, wherein the compound is N-{[4-(2-tert-Butylphenyl)piperazin-1-yl]carbonyl}glycine, 3-[4-(2-tert-Butylphenyl)piperazin-1-yl]-3-oxopropanoic acid, [4-(2-tert-Butyl-4-chlorophenyl)piperazin-1-yl](oxo)acetic acid, 5-{2-[4-(2-tert-Butylphenyl)piperazin-1-yl]-2-oxoethyl}imidazolidine-2,4-dione, [(5-{[4-(2-tert-Butylphenyl)piperazin-1-yl]carbonyl}isoxazol-3-yl)oxy]acetic acid, or a pharmaceutically acceptable salt thereof.

88. The method of claim 33, 45, 53, 54, 79, or 80, wherein the pharmaceutical composition comprises a compound having the structure of Formula (IV): 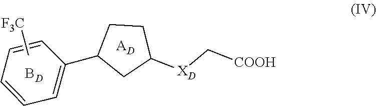
wherein
ring A_{D} is a 5-membered non-aromatic heterocycle optionally further substituted by one oxo group;
ring B_{D} is a benzene ring optionally further substituted by 1 to 4 substituents; and
X_{D} is O, CH₂O, OCH₂, CH₂, (CH₂)₂, S, CH₂S, SCH₂, S(O), CH₂S(O), S(O)CH₂, S(O)₂, CH₂S(O)₂ or S(O)₂CH₂,
or a pharmaceutically acceptable salt thereof.

89. The method of claim 88, wherein the compound is ({(3S)-1-[3,5-Bis(trifluoromethyl)phenyl]pyrrolidin-3-yl}oxy)acetic, ({1-[4-Chloro-3-(trifluoromethyl)phenyl]pyrrolidin-3-yl}sulfanyl)acetic acid, 3-{(2R,5S)-5-[3,5-Bis(trifluoromethyl)phenyl]tetrahydrofuran-2-yl}propanoic acid, or a pharmaceutically acceptable salt thereof.

90. The method of claim 33, 45, 53, 54, 79, or 80, wherein the pharmaceutical composition comprises a compound having the structure of Formula (V): 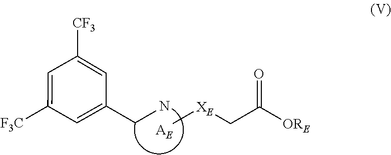
wherein
ring A_{E} is a pyrazole ring, a pyridine ring, an oxazole ring, an imidazole ring, or a pyrimidine ring;
X_{E} is S, optionally substituted alkylene, or O; and
R_{E} is a hydrogen atom or a C₁-C₆alkyl group,
or a pharmaceutically acceptable salt thereof.

91. The method of claim 90, wherein the compound is ((4-(3,5-bis(trifluoromethyl)phenyl)-1,3-oxazol-2-yl)sulfanyl)acetic acid, ethyl ((6-(3,5-bis(trifluoromethyl)phenyl)-pyridin-3-yl)sulfanyl)acetic acid, ((6-(3,5-bis(trifluoromethyl)-phenyl)pyridine-3-yl)sulfanyl)acetic acid, or 3-(3-(3,5-bis(trifluoromethyl)phenyl)-1H-pyrazol-1-yl)butanoic acid.
